(19) United States
(12) Patent Application Publication
(10) Pub. No.: US 2025/0186546 A1
(43) Pub. Date: Jun. 12, 2025

(54) Compositions and Methods to Prevent, Inhibit or Treat Autoimmune and Vascular Disease

(71) Applicants: Katherine R. CROCE, New York, NY (US); Al YAMAMOTO, New York, NY (US); Xun WU, New York, NY (US); Hanrui ZHANG, New York, NY (US)

(72) Inventors: Katherine R. CROCE, New York, NY (US); Al YAMAMOTO, New York, NY (US); Xun WU, New York, NY (US); Hanrui ZHANG, New York, NY (US)

(21) Appl. No.: 18/747,983

(22) Filed: Jun. 19, 2024

Related U.S. Application Data
(60) Provisional application No. 63/521,837, filed on Jun. 19, 2023.

(51) Int. Cl. A61K 38/17; A61K 48/00; A61P 37/06

(52) U.S. Cl. CPC A61K 38/1709; A61K 48/0033; A61P 37/06

(57) ABSTRACT

Provided here are methods to prevent, inhibit or treat an autoimmune disease or a vascular or cardiovascular disorder in a mammal, comprising administering to the mammal a composition comprising an effective amount of isolated nucleic acid comprising a nucleotide sequence encoding WDFY3/Alfy or a portion thereof, a nucleotide sequence comprising a WDFY3/Alfy-specific long non-coding RNA (lncRNA) or a corresponding DNA sequence, a nucleotide sequence comprising a portion of a lncRNA WDFY3-AS2 or a corresponding DNA sequence, or isolated WDFY3/Alfy or a portion thereof.

### PRIORITY

This application claims the benefit of U.S. Provisional Application No. 63/521,837, filed Jun. 19, 2023, the content of which is herein incorporated by reference in its entirety.

### STATEMENT OF GOVERNMENT RIGHTS

This invention was made with government support under grants HL151611, NS077111, and NS101663 awarded by the National Institutes of Health. The government has certain rights in the invention.

### BACKGROUND

Phagocytic clearance of dead or dying cells by phagocytes, a process known as efferocytosis, is important in embryogenesis and development, and the resolution of pathological events (Boada-Romero et al., 2020; Doran et al., 2019; Morioka et al., 2019; Trzeciak et al., 2021). Impaired efferocytosis lessens the effective clearance of dying cells, causing secondary necrotic cell death and damages (Boada-Romero et al., 2020; Doran et al., 2019; Morioka et al., 2019; Trzeciak et al., 2021). Efferocytosis is performed by macrophages and to a lesser extent by other professional phagocytes (such as monocytes and dendritic cells), non-professional phagocytes and specialized phagocytes (Boada-Romero et al., 2020). Because of the fundamental role of efferocytosis, dysregulation of this process is associated with many pathological states, including autoimmune diseases, atherosclerosis, and cancers (Doran et al., 2019). Given the importance of this biological process and the therapeutic potential of targeting genes regulating efferocytosis, identifying novel regulators and mechanisms of this biological process has broad impacts on many diseases relevant to defective process has broad impacts on many diseases relevant to defective efferocytosis (Greenlee-Wacker, 2016; Kojima et al., 20171 Yurdagul et al., 2017; Hayat et al., 2020).

Hypothesis-driven approaches have successfully identified many key regulators for the removal of dying cells via efferocytosis (Boada-Romero et al., 2020; Doran et al., 2019; Morioka et al., 2019; Trzeciak et al., 2021). Yet, an unbiased approach to screening regulators of efferocytosis of apoptotic cells (ACs) on a genome-wide scale is lacking. Unbiased screenings allow the identification of new regulators from diverse and unexpected gene classes. Genetic screens of efferocytosis of ACs have been performed in Drosophila (Silva et al., 2007), but not in mammalian cells. In mammalian cells, genome-wide CRISPR knockout screens have identified regulators of diverse substrates in differentiated myeloid leukemia cells (Sedlyarov et al., 2018; Haney et al., 2018) and macrophage-like cells (Kamber et al., 2021), illuminating both universal and specific principles of phagocytosis, but not of ACs. However, a screening platform using ACs as the substrates and in primary macrophages is important because efferocytosis involves AC-specific recognition receptors (Penberthy & Ravichandran, 2016), stiffness and size-dependent engulfment mechanisms (Schlam et al., 2015), and cellular response to degradation (Trzeciak et al., 2021), all of which cannot be recapitulated by phagocytosis of beads. In addition, immortalized or tumor-derived monocytic cell lines often lack physiological relevance to resemble fully the spectrum of physiological function in primary macrophages (Andreu et al., 2017).

### SUMMARY

A pooled genome-wide CRISPR knockout screen was established for efferocytosis in primary murine bone marrow-derived macrophages (BMDMs) derived from the Rosa26-Cas9 knock-in mice constitutively expressing Cas9 endonuclease. The screen successfully identified w key regulators responsible for the recognition and uptake of ACs, supporting the screen's performance. Individual validation of the strong hits uncovered WDFY3 (WD repeat and FYVE domain containing 3), also known as Alfy (Autophagy-linked FYVE Protein), as a novel regulator previously not implicated in the regulation of efferocytosis or phagocytosis. The mechanisms by which WDFY3 regulates the uptake and degradation of ACs during efferocytosis were identified and the role of WDFY3-mediated efferocytosis was demonstrated in vivo in mice and in vitro in primary human macrophages. The study also establishes a broadly applicable platform for the genome-wide screen of complex functional phenotypes in primary macrophages for unbiased discoveries.

The disclosure provides methods useful to prevent, inhibit or treat an autoimmune disease in a mammal. Autoimmune diseases include but are not limited to Addison disease, celiac disease—sprue (gluten-sensitive enteropathy), dermatomyositis, Graves disease, Hashimoto thyroiditis, multiple sclerosis, myasthenia gravis, pernicious anemia, reactive arthritis, rheumatoid arthritis, Sjögren syndrome, systemic lupus erythematosus, or Type I diabetes. Examples of other autoimmune diseases include, but are not limited to, ankylosing spondylitis, autoimmune cardiomyopathy, autoimmune hemolytic anemia, autoimmune hepatitis, autoimmune inner ear disease, immune lymphoproliferative syndrome, autoimmune peripheral neuropathy, autoimmune pancreatitis, autoimmune polyendocrine syndrome, autoimmune thrombocytopeniarpura, cold agglutinin disease, contact dermatitis, Crohn's disease, Diabetes mellitus type 1, eosinophilic fasciitis, gastrointestinal pemphigoid, Goodpasture's syndrome, Guillain-Barré syndrome, Hashimoto's encephalopathy, idiopathic thrombocytopeniaurpura, Miller-Fisher syndrome, pemphigus vulgaris, polymyositis, primary biliary cirrhosis, psoriasis, psoriatic arthritis, relapsing polychondritis, temporal arteritis, transverse myelitis, ulcerative colitis, undifferentiated connective tissue disease, vasculitis, vitiligo, or Wegener's granulomatosis. The method comprises administering to a mammal at risk of or having an autoimmune disease a composition an effective amount of isolated nucleic acid encoding WDFY3/Alfy or a portion thereof, e.g., a vector comprising a nucleotide sequence encoding WDFY3/Alfy or a portion thereof, isolated WDFY3/Alfy or a portion thereof, or a long non-coding RNA (lncRNA) of WDFY3/Alfy or a portion thereof, or a corresponding DNA. In one embodiment, WDFY3/Alfy is a WDFY3/Alfy variant, e.g., having one or more substitutions, e.g., at least one amino acid substitution at a position from 3025 to 3037 in human WDFY3/Alfy, e.g., position 3032 has a valine, or nucleic acid encoding a variant. In one embodiment, overexpression of WDFY3/Alfy or a portion thereof, such as C-terminal WDFY3/Alfy, which may include residues 2461 to 3526 and/or a BEACH and FYVE domain, or nucleic acid encoding WDFY3/Alfy or a portion thereof or comprising a sequence for a lncRNA such as WDFY3-AS2 or a portion thereof, in a mammal, such as a human, may prevent, inhibit or treat one or more symptoms of the autoimmune disease. In one embodiment, the vector is a viral vector or a set of vectors, e.g., a viral vector or a set of viral vectors comprising adeno-associated virus, adenovirus, lentivirus or a herpesvirus vector(s). In one embodiment, the isolated nucleic acid comprises a lncRNA of WDFY3/Alfy or a portion thereof or a corresponding DNA sequence. In one embodiment, the composition is systemically administered. In one embodiment, the isolated nucleic acid comprises DNA. In one embodiment, a heterologous promoter is operably linked to DNA encoding WDFY3/Alfy or a portion thereof. In one embodiment, the isolated nucleic acid comprises RNA. In one embodiment, the DNA or RNA comprises a plurality of modified nucleotides. In one embodiment, the composition comprises liposomes or nanoparticles. In one embodiment, the composition is a sustained release composition.

The disclosure also provides methods useful to prevent, inhibit or treat vascular disease in a mammal. Vascular diseases include but are not limited to peripheral vascular disease, limb-threatening ischemia, carotid artery disease, abdominal aortic aneurysm, chronic venous insufficiency, varicose veins, deep vein thrombosis, or pulmonary embolism or atherosclerosis related vascular diseases. The method comprises administering to a mammal having or at risk of having a vascular disease or event, a composition an effective amount of isolated nucleic acid encoding WDFY3/Alfy or a portion thereof, e.g., a vector comprising a nucleotide sequence encoding WDFY3/Alfy or a portion thereof, isolated WDFY3/Alfy or a portion thereof, or a lncRNA of WDFY3/Alfy or a portion thereof, or a corresponding DNA. In one embodiment, overexpression of WDFY3/Alfy or a portion thereof, such as C-terminal WDFY3/Alfy, which may include residues 2461 to 3526 and/or a BEACH and FYVE domain, in a mammal, such as a human, may prevent, inhibit or treat one or more symptoms of the vascular disease. In one embodiment, the vector is a viral vector or set of vectors, e.g., a set of viral vectors comprising adeno-associated virus, adenovirus, lentivirus or a herpesvirus. In one embodiment, the isolated nucleic acid comprises a lncRNA), a portion thereof or a corresponding DNA sequence. In one embodiment, the composition is systemically administered. In one embodiment, the isolated nucleic acid comprises DNA. In one embodiment, a heterologous promoter is operably linked to DNA encoding WDFY3/Alfy or portion thereof. In one embodiment, the isolated nucleic acid comprises RNA. In one embodiment, the RNA or DNA comprises a plurality of modified nucleotides. In one embodiment, the composition comprises liposomes or nanoparticles. In one embodiment, the composition is a sustained release composition. In one embodiment, C-terminal WDFY3/Alfy, may include residues 2400 to 3526 of SEQ ID NO: 1 or SEQ ID NO: 2, or a polypeptide having at least 250, 300, 350, 400, 450, 500, 550, 600, 650, 700, 750, 800, 850, 900, 950, 1000 or 1100 amino acids thereof, or any integer in between 250 to 1126, or a polypeptide with at least 80%, 90%, 92%, 94%, 95%, 96%, 97%, 98%, 99% or more amino acid sequence identity thereto.

The disclosure provides methods useful to prevent, inhibit or treat a cardiovascular disease in a mammal. Cardiovascular diseases include but are not limited to heart disease, e.g., atherosclerosis, heart attack (myocardial infarction), stroke, e.g., an ischemic stroke or a hemorrhagic stroke, hypertension (high blood pressure), heart failure, arrhythmia, e.g., bradycardia, or tachycardia, or heart valve problems. The method comprises administering to a mammal having or at risk of having a cardiovascular event, a composition an effective amount of isolated nucleic acid encoding WDFY3/Alfy or a portion thereof, e.g., a vector comprising a nucleotide sequence encoding WDFY3/Alfy or a portion thereof, or isolated Alfy or a portion thereof, WDFY3/Alfy or a portion thereof, a lncRNA of WDFY3/Alfy or a portion thereof. In one embodiment, overexpression of WDFY3/Alfy or a portion thereof, such as C-terminal WDFY3/Alfy, which may include residues 2461 to 3526 and/or a BEACH and FYVE domain, in a mammal, such as a human, may prevent, inhibit or treat one or more symptoms of the cardiovascular disease. In one embodiment, the vector is a viral vector or set of vectors, e.g., a set of viral vectors comprising adeno-associated virus, adenovirus, lentivirus or a herpesvirus. In one embodiment, the isolated nucleic acid comprises a lncRNA), a portion thereof or a corresponding DNA sequence. In one embodiment, the composition is systemically administered. In one embodiment, the isolated nucleic acid comprises DNA. In one embodiment, a heterologous promoter is operably linked to DNA encoding WDFY3/Alfy or portion thereof. In one embodiment, the isolated nucleic acid comprises RNA. In one embodiment, the RNA comprises a plurality of modified nucleotides. In one embodiment, the composition comprises liposomes or nanoparticles. In one embodiment, the composition is a sustained release composition.

In one embodiment, a lnc RNA sequence, e.g., WDFY3-AS2 or a portion thereof, or a corresponding DNA sequence, is employed in the methods to regulate WDFY3/Alfy expression. In one embodiment, the portion is a portion of a lncRNA, such as WDFY3-AS2, e.g., a portion of SEQ ID NO: 3 including RNA, DNA or modified forms thereof, e.g., having nucleotide analogs. In one embodiment, the portion is less than 90%, 85%, 80%, 75%, 70%, 65%, 60%, 55%, 50%, 45%, 40%, 35%, 30%, 28%, 25%, 22%, 20%, 18%, 15%, 12%, 10%, 7%, 5%, 4%, 3%, 2%, 1%, 0.6%, 0.3% 0.1% or less than the length of SEQ ID NO: 3. In one embodiment, the portion is a portion of SEQ ID NO: 3, for instance, from 100 to 150, 110 to 130, 120 to 140, 130 to 160, 150 to 250, 200 to 300, 300 to 400, 400 to 500, 500 to 600, 600 to 700, 700 to 800, 800 to 900 nucleotides or more of SEQ ID NO: 3. In one embodiment, the portion has TCTTTCCTTACTTCCTTCCCTTCCCTCGGCTTCCCGCTCTTGCCTCACTC (SEQ ID NO: 11), or a portion thereof, or a nucleotide sequence with at least 80%, 85%, 88%, 90%, 92%, 94%, 95%, 96%, 97%, 98% or 99% nucleic acid sequence identity thereto. In one embodiment, the portion has TCCCACCCCTGCCCCTCT (SEQ ID NO: 12), or a portion thereof, or a nucleotide sequence with at least 80%, 85%, 88%, 90%, 92%, 94%, 95%, 96%, 97%, 98% or 99% nucleic acid sequence identity thereto. In one embodiment, the portion has AACTGGGCAAGCCAATGAAAGTCTCTGGGGAT (SEQ ID NO: 13), or a portion thereof, or a nucleotide sequence with at least 80%, 85%, 88%, 90%, 92%, 94%, 95%, 96%, 97%, 98% or 99% nucleic acid sequence identity thereto. In one embodiment, the portion has GGAAGGCTGGGA (SEQ ID NO: 14), or a portion thereof, or a nucleotide sequence with at least 80%, 85%, 88%, 90%, 92%, 94%, 95%, 96%, 97%, 98% or 99% nucleic acid sequence identity thereto. In one embodiment, the isolated nucleic acid may include two or more of SEQ ID NO: 11, or a portion thereof, or a nucleotide sequence with at least 80%, 85%, 88%, 90%, 92%, 94%, 95%, 96%, 97%, 98% or 99% nucleic acid sequence identity thereto; SEQ ID NO: 12, or a portion thereof, or a nucleotide sequence with at least 80%, 85%, 88%, 90%, 92%, 94%, 95%, 96%, 97%, 98% or 99% nucleic acid sequence identity thereto; SEQ ID NO: 13, or a portion thereof, or a nucleotide sequence with at least 80%, 85%, 88%, 90%, 92%, 94%, 95%, 96%, 97%, 98% or 99% nucleic acid sequence identity thereto; SEQ ID NO: 14, or a portion thereof, or a nucleotide sequence with at least 80%, 85%, 88%, 90%, 92%, 94%, 95%, 96%, 97%, 98% or 99% nucleic acid sequence identity thereto; or any combination thereof, in any linear order. In one embodiment, the isolated nucleic portion has at least 5, 10, 15, 20, 25, 30, 35, 40 or more contiguous nucleotides of SEQ ID NO: 11. In one embodiment, the isolated nucleic portion has at least 5, 6, 7, 8, 9, 10, or 15 or more contiguous nucleotides of SEQ ID NO: 12. In one embodiment, the isolated nucleic portion has at least 5, 10, 15, 20, 25, 30 or more contiguous nucleotides of SEQ ID NO: 13. In one embodiment, the isolated nucleic portion has at least 5, 6, 7, 8, 9, or 10 or more contiguous nucleotides of SEQ ID NO: 14. Thus, expression or overexpression of a portion of the lncRNA WDFY3-AS2 may be sufficient to prevent, inhibit or treat one or more symptoms of diseases including autoimmune disease, vascular disease or cardiovascular disease.

The disclosure thus provides a method to enhance efferocytosis in a mammal having an autoimmune disease, a vascular disease or a cardiovascular disease. The method includes administering to the mammal a composition comprising an effective amount of isolated nucleic acid for expression of, for example, lncRNA WDFY3-AS2 or a portion of the lncRNA WDFY3-AS2 including a vector comprising a nucleotide sequence comprising lncRNA WDFY3-AS2 or a portion of the lncRNA WDFY3-AS2. In one embodiment, the mammal is a human. In one embodiment, the vector is a viral vector. In one embodiment, the viral vector comprises an adeno-associated virus, adenovirus, lentivirus or a herpesvirus. In one embodiment, the composition is systemically administered. In one embodiment, the isolated nucleic acid comprises DNA. In one embodiment, a heterologous promoter is operably linked to DNA for expression of the portion. In one embodiment, the isolated nucleic acid comprises RNA. In one embodiment, the RNA comprises a plurality of modified nucleotides. In one embodiment, the composition comprises liposomes or nanoparticles. In one embodiment, the composition is a sustained release composition. In one embodiment, the nucleic acid comprises modified bases, modified sugars or modified phosphate linkages, or a combination thereof. In one embodiment, the composition is locally administered. In one embodiment, the composition is systemically administered. In one embodiment, the isolated nucleic acid comprises any one of SEQ ID NOs: 11-14, or a combination thereof, or a nucleotide sequence with at least 80% nucleotide sequence identity thereto. In one embodiment, the isolated nucleic acid comprises a nucleotide sequence with at least 90% nucleotide sequence identity to any one of SEQ ID NOs: 11-14. In one embodiment, the isolated nucleic acid comprises a portion of any one of SEQ ID NOs: 11-14 with the same activity as one of SEQ ID NOs: 11-14. In one embodiment, the isolated nucleic acid comprises a plurality of modified nucleotides. In one embodiment, the isolated nucleic acid is operably linked to a heterologous promoter. In one embodiment, the composition comprises liposomes or nanoparticles. In one embodiment, the portion is in the first 1000 nucleotides of SEQ ID NO: 3. In one embodiment, the portion is in the first 500 nucleotides of SEQ ID NO: 3. In one embodiment, the portion is in the last 500 nucleotides of SEQ ID NO: 3. In one embodiment, the portion is in the first 250 nucleotides of SEQ ID NO: 2. In one embodiment, the portion is in the last 250 nucleotides of SEQ ID NO:2. In one embodiment, the nucleic acid comprises RNA. In one embodiment, the nucleic acid comprises DNA. In one embodiment, the composition comprises nanoparticles comprising the nucleic acid. In one embodiment, the composition comprises liposomes comprising the nucleic acid.

### BRIEF DESCRIPTION OF THE FIGURES

FIGS. 1A-1G. A pooled, fluorescence-activated cell sorting (FACS)-based genome-wide CRISPR knockout screen in primary mouse macrophages identified known and novel regulators of macrophage efferocytosis. A) Schematics of the CRISPR screen workflow. B) Timeline of bone marrow isolation, lentiviral library transduction, puromycin selection, efferocytosis, and cell sorting. C) Visualization of gating strategy for separation of non-eaters and efficient eaters. Successful separation was confirmed by fluorescent microscopy. D) Volcano plot highlights the top-ranked screen hits that are known positive and negative regulators of macrophage efferocytosis. e Canonical pathways enriched in top-ranked positive regulators by Ingenuity Pathway Analysis (IPA). F) Canonical pathways enriched in top-ranked negative regulators by IPA. G) Validation of Wdfy3 as a positive regulator required for macrophage efferocytosis (n=4 independent experiments). Data are presented as mean±SEM. Two-sided P values were determined by a two-way ANOVA with Tukey's multiple comparisons test in panel G) ACs, apoptotic cells; BMDMs, bone marrow-derived macrophages.

FIGS. 2A-2H. WDFY3 deficiency led to impaired uptake, as opposed to binding, of apoptotic cells (ACs) due to defective actin depolymerization. A) Schematics of breeding LysMCre mice with Wdfy3^{fl/fl} mice to obtain mice with myeloid-specific knockout of Wdfy3. B) Validation of efficient knockout in BMDMs by western blot of WDFY3 (n=4 biological replicates; the blot shown is a representative image of three independent experiments). C) Cre⁻ and Cre⁺ BMDMs were incubated with Hoechst-labeled ACs at various AC:BMDM ratios of 3:1, 5:1, 10:1 respectively for 1 hour and analyzed by flow cytometry (n=3 biological replicates, each from the average of 2 technical replicates). D) Cre⁻ and Cre⁺ BMDMs were incubated with PKH26-labeled ACs at various time points of 15 minutes, 30 minutes, and 60 minutes at a AC:BMDM ratio of 5:1 and analyzed by flow cytometry (n=3 technical replicates). E) Cre⁻ and Cre⁺ BMDMs were pretreated with cytochalasin D for 30 minutes to block polymerization and elongation of actin, thus testing the binding of ACs with BMDMs. The treated BMDMs were then incubated with TAMRA-stained apoptotic mouse thymocytes at 37° C. for 30 minutes and then extensively washed with DPBS to remove unbound ACs for imaging and quantification after fixation (n=6 biological replicates). F) Cre⁻ and Cre⁺ BMDMs were stained with CellTracker and incubated with ACs. Efferocytosis of ACs by BMDMs were observed using time-lapse confocal microscopy. The time required for phagosome formation was recorded and quantified (n=44 and 47 data points for Cre⁻ and Cre⁺ respectively, each data point represents one BMDM with engulfed ACs, 4 biological replicates for each genotype). The arrows point to the BMDM engulfing an AC across the stages from phagocytic cup formation to phagosome closure (from left to right). G) F-actin labeled by siR-actin in Cre⁻ and Cre⁺ BMDMs was quantified by flow cytometry (n=4 biological replicates, each from the average of 3 technical replicates). H) BMDMs were stained with CellTracker and siR-actin, then incubated with Nuclear Mask Blue labeled apoptotic Jurkat cells for various time points (10 minutes, 20 minutes, 40 minutes, and 60 minutes). For each time point, unbound ACs were removed and BMDMs were fixed. BMDMs were imaged and the percentage of BMDMs with engulfed cargos surrounded by F-actin rings in all BMDMs with engulfed cargos was quantified (n=4 biological replicates, data are representative of two independent experiments). Data are presented as mean±SEM. Two-sided P values were determined by a two-way ANOVA with Tukey's multiple comparisons test in (C, D, E, G, H), or by unpaired t test in panel f.

FIGS. 3A-3G. WDFY3 deficiency led to defects in LC3-associated phagocytosis (LAP) and the degradation of engulfed ACs. A) Cre⁻ and Cre⁺ BMDMs were incubated with PKH26-labeled ACs for 1 hour. After washing away the unengulfed ACs, BMDMs were placed back to the incubator for another 3 hours. BMDMs were then fixed and imaged. The percentage of BMDMs showing non-fragmented PKH26 signals in the total number of PKH26⁺ BMDMs was quantified (n=4 and 5 biological replicates for Cre⁻ and Cre⁺ respectively, each from the average of 3 technical replicates). b Cre⁻ and Cre⁺ BMDMs were incubated with TAMRA-labeled ACs for 1 hour. After washing away the unengulfed ACs, BMDMs were either collected for flow cytometry to quantify the MFI of TAMRA or placed back to the incubator for another 16 hours and then collected for flow cytometry. The rate of degradation was calculated, as shown in the schematics (n=8 and 9 biological replicates for Cre⁻ and Cre⁺ respectively). C) Cre⁻ and Cre⁺ BMDMs were incubated with ACs labeled by Hoechst, which stains DNA and is pH-insensitive, and pHrodo, which is pH-sensitive and shows fluorescent signal only under an acidified environment in the phagolysosome. The percentage of Hoechst⁺ BMDMs indicates uptake. The percentage of Hoechst⁺/pHrodo+BMDMs in Hoechst⁺ BMDMs indicates acidification of the engulfed cargos (n=8 biological replicates, each from the average of 2 technical replicates). D) Schematics of known functional domains and binding partners of human WDFY3. E) The interaction between WDFY3 and GABARAP was assessed by co-immunoprecipitation. Cre⁻ and Cre⁺ BMDM cell lysates were incubated with anti-GABARAP antibody and protein A/G agarose beads. Beads-bound proteins were detected with anti-WDFY3 antibodies (n=3 independent experiments with similar results). HC refers to heavy chain. F) Cre⁻ and Cre⁺ BMDMs were incubated with ACs for 1 hour. Unbound ACs were washed away and BMDMs were collected for measurement of LC3-II by western blot (n=5 biological replicates). The image shows the representative blot. * denotes non-specific band). G) BMDMs were incubated with Hoechst-labeled ACs to allow efferocytosis. After removal of unbound ACs, BMDMs were collected and treated with digitonin to remove non-membrane bound LC3, and then immunostained for LC3 that is lipidated and membrane bound. LC3-II staining was then quantified by flow cytometry for BMDMs that had engulfed Hoechst-labeled ACs (n=5 biological replicates). Data are presented as mean±SEM in (B, C, F), or as median±95% CI in (A, G). Two-sided P values were determined by a two-way ANOVA with Tukey's multiple comparisons test in (B, C, F), or by Mann-Whitney test in (A, G).

FIGS. 4A-4E. The C-terminal WDFY3 is sufficient for regulating degradation yet the full-length WDFY3 is required for the uptake of ACs during efferocytosis. A) Schematics of lentiviral overexpression of C-terminal WDFY3 in BMDMs of Cre and Cre⁺ mice. B) C-terminal WDFY3 did not restore uptake, yet partially rescued the defects in cargo acidification in Cre⁺ mice (n=7 biological replicates, each from the average of 2 technical replicates). C) C-terminal WDFY3 restored LC3-II levels in Cre⁺ mice as determined by western blot (n=5 biological replicates. * indicates non-specific bands) and in (d) by flow cytometry (n=3 biological replicates). E) BMDMs from GFP-LC3 mice were transfected with tdTomato-fused C-terminal WDFY3 (2981-3526) plasmid via electroporation. BMDMs were fed with Hoechst-labeled ACs. Unengulfed ACs were washed away and BMDMs were imaged to visualize GFP-LC3 phagosome association, C-WDFY3 intracellular localization, and GFP-LC3/tdTomato-WDFY3 colocalization with and without AC engulfment (n=21 cells from 5 biological replicates. Images are representatives of 5 independent experiments each using one GPF-LC3 mouse). Data are presented as mean±SEM. Two-sided P values were determined by a two-way ANOVA with Tukey's multiple comparisons test in (B-D) or by unpaired t test in (E).

FIGS. 5A-5H. Mice with myeloid Wdfy3 knockout show impaired efferocytosis in vivo. A) Schematics of experimental design for in vivo thymus efferocytosis assay. B) Thymus weight. C) Total number of cells per thymus. D) Percentage of F4/80⁺ macrophages in the thymus determined by flow cytometry. E) Percentage of Annexin V⁺ ACs per thymus determined by flow cytometry. A higher percentage implies impaired efferocytic clearance. F) Thymic sections were stained with TUNEL for ACs, and CD68 for macrophages. The ratio of macrophage associated TUNEL⁺ cells vs. free TUNEL⁺ cells was quantified and summarized. The white and yellow squares highlight the macrophage associated and free TUNEL⁺ cells, respectively (n=5 biological replicates). G) Schematics of experimental design for in vivo peritoneal macrophage efferocytosis assay. H) Peritoneal exudates were stained for F4/80 and the percentage of TAMRA⁺ peritoneal macrophages was determined by flow cytometry (n=5 biological replicates). n=9 Cre⁻ and 9 Cre⁺ biological replicates for PBS group, n=10 Cre⁻ and 14 Cre⁺ biological replicates for Dexamethasone group in (B, C, E). n=8 Cre⁻ and 7 Cre⁺ biological replicates for PBS group, n=9 Cre⁻ and 12 Cre⁺ biological replicates for Dexamethasone group in (D). Data are presented as mean±SEM in (B-E), as median±95% CI in (F) and (H). Two-sided P values were determined by a two-way ANOVA with Tukey's multiple comparisons test in (B-E), or by Mann-Whitney test in (F) and (H).

FIGS. 6A-6G. WDFY3 regulates efferocytosis in human macrophages. A) Schematics of human monocyte differentiation to macrophages (HMDMs) and knockdown of WDFY3 with Lipofectamine RNAiMAX-mediated transfection of siRNAs targeting WDFY3, or non-targeting siRNAs as the control. B) Validation of knockdown efficiency at mRNA level by qRT-PCR (n=4 independent experiments, each from the average of 3 technical replicates). C) Validation of knockdown efficiency at protein level by western blot (n=2 biological replicates, data are representative of 3 independent experiments). D) Efferocytosis of apoptotic Jurkat cells labeled by both Hoechst and pHrodo. The percentage of HMDMs with Hoechst-labeled ACs (indicating uptake), and the percentage of Hoechst⁺/pHrodo⁺ HMDMs in Hoechst⁺ HMDMs (indicating acidification upon uptake) were quantified by flow cytometry. Both uptake and acidification of ACs were impaired in HMDMs with siRNA mediated WDFY3 knockdown (n=4 independent experiments, each from the average of 2 technical replicates). E) Fragmentation of engulfed ACs was assessed 3 hours after washing away the unengulfed ACs. The percentage of HMDMs with non-fragmented PKH26 staining in all PKH26⁺ HMDMs was determined (n=4 independent experiments). F) Flow cytometry-based degradation assay was performed in HMDMs with procedures as described in FIG. 3B (n=5 independent experiments). G) RNA-seq was performed for HMDMs either unstimulated (MO) or treated with LPS and IFNγ for 18-20 hours (M1-like). The expression of WDFY3 was visualized (n=48 biological replicates). The box shows Q1, median, and Q3; the whiskers show 1.5× interquartile range, though the lower whiskers in this plot only extend to the minimum as the minimum values are greater than the values corresponding to the lower whiskers. There is one outlier in MO with FPKM 7.404. There are two outliers in M1-like with FPKMs 5.943 and 4.345. Data are presented as median±95% CI. Two-sided P values were determined by Mann-Whitney test.

FIGS. 7A-7C. A) Schematic figure summarizing how WDFY3 regulates macrophage efferocytosis. WDFY3 is a regulator of efferocytosis by macrophages. WDFY3 deficiency in macrophages specifically impaired uptake, not binding, of apoptotic cells due to defective actin depolymerization, thus phagosome formation. WDFY3 directly interacts with GABARAP, one of the seven members of the LC3/GABARAP protein family, to facilitate LC3 lipidation and the subsequent phagosome-lysosome fusion and degradation of the engulfed AC components. B) WDFY3 is important for the degradation of cargos by facilitating LC3-mediated autophagosome-lysosome fusion. C) Overexpressing C-WDFY3 (2543-3526) rescues the acidification, but not uptake of ACs Wdfy3−/− BMDM.

FIGS. 8A-8B. Known functional domains of WDFY3 (A) and macrophage-enriched expression of WDFY3 shown in Human Protein Atlas (B). Cell types in spleen and bone marrow.

FIGS. 9A-9D. Wdfy3 knockout impairs uptake of ACs (A), but not zymosan (B) or latex beads (C-D). Data are shown as mean±SEM. n=6 mice. Statistical tests were performed according to the statistical methods section in Example 2. AC, apoptotic cells. Cre−, LysMCre^{-/-}Wdfy3^{fl/fl}, Cre+, LysMCre^{+/-}Wdfy3^{fl/fl}.

FIGS. 10A-10D. Wdfy3 knockout impairs, while overexpression enhances efferocytosis of dying cells in vitro and in vivo. (A) Myeloid-knockout of Wdfy3 led to impaired uptake, acidification, and LC3 lipidation; (B) hWDFY3^{Tg} enhanced uptake, acidification, and LC3 lipidation; (C) in vivo efferocytosis by peritoneal macrophages; (D) siRNA knockdown of WDFY3 in human macrophages led to impaired uptake and acidification. Data are shown as mean±SEM. n=number of mice/subjects. Statistical tests were performed according to the statistical methods section in Example 2. AC, apoptotic cells.

FIGS. 11A-11B. Proximity labeling by TurboID identifies proximal proteins to C-WDFY3. (A) Schematics of TurboID, an engineered biotin ligase that uses ATP to convert biotin into biotin-AMP, a reactive intermediate that covalently labels proximal proteins (about 20 nm zone). Labeled biotinylated proteins are pulled down for mass spectrometry. (B) Lenti-TurboID-WDFY3₍₂₅₄₃₋₃₅₂₆₎ construct was transduced to BMDM for biotin labeling of proximal proteins in BMDMs with or without AC engulfment. Data were analyzed and visualized by Perseus software using ANOVA followed by Tukey post hoc test to nominate the top hits as shown. Arp2/3 complex; Myosins; proteins with literature supporting roles in regulating F-actin dynamics.

FIGS. 12A-12B. A High-Content Imaging Analysis (HCA)-based platform for arrayed efferocytosis assays. (A) Automated imaging analysis precisely identifies macrophages that engulfed apoptotic cells (★) vs macrophages that did not engulf (#). (B) HCA and FACS-based analyses showed consistent results. Cyto D, cytochalasin D (inhibiting actin polymerization thus efferocytosis). n=4 mice.

FIGS. 13A-13B. Engulfment of ACs with increased rigidity does not require WDFY3. Glutaraldehyde (GA) treatment (10 nM, 1 minute) increases cell rigidity by crosslinking proteins. BMDMs show increased engulfment of the rigid ACs. n=3 mice.

FIGS. 14A-14B. Hypotheses based on proximal proteins identified by TurboID-WDFY3 proximity labeling and initial validation supporting interaction between C-WDFY3 and Arp2/3 complex with/without efferocytosis. (A) Crystal structure of Arp2/3 complex. ARP2, ARP3, ARPC1, and ARPC4 were identified as proximal proteins to WDFY3. (B) Pull-down validation supports direct interaction between endogenous ARP2 and WDFY3 in BMDMs. n=4 experiments.

FIG. 15. Feasibility of tagging endogenous WDFY3 by CRISPR-mediated knock-in of eGFP for live-cell imaging in THP-1 derived macrophages. The signals support cytoplasmic localization.

FIGS. 16A-16D. Wdfy3 knockout BMDM show downregulation of genes in “ROS and RNS production in phagocytes” pathway (A-B), and lower mitochondrial ROS production upon AC stimulation (C). (A) Gene-Set Enrichment Analysis and heatmap to visualize the transcript abundance of the top downregulated genes contributing to the pathway enrichment; (B) qPCR validation; (C) MitoSox; (D) roGFP can measure reductive state as confirmed by treatment of reducing reagent Dithiothreitol (DTT) as a positive control. n=number of mice. mean±SEM. Statistical tests performed according to the statistical methods section.

FIG. 17. TAMRA-labeled apoptotic thymocytes injection led to accumulation in major organs. Wdfy3 deficiency led to increased accumulation and delayed clearance in the kidney, but not the liver or spleen. i.v. injection of 1×10\hat{ }7 ACs. n=3 mice (males and females combined). mean±SEM.

FIGS. 18A-18D. With chronic injection of apoptotic thymocytes, Wdfy3 knockout mice showed increased ANA (A), kidney immune complex deposition (B-C) and increased circulating IL-18 (D). Apoptotic thymocytes (1×10\hat{ }7) were injected i.v. weekly for 8 weeks. n=the number of mice (males and females combined). mean±SEM.

FIGS. 19A-19B. hWDFY3T8 protects the mice against chronic apoptotic thymocytes injection induced autoimmunity as shown by lower anti-ANA (A) and anti-dsDNA (B). n=6-7 mice (males and females combined). mean±SEM.

FIG. 20. Myeloid-specific Wdfy3 knockout and lupus-like pathological changes after chronic AC injection.

FIG. 21. Myeloid-specific Wdfy3 knockout and exacerbation of impaired efferocytosis and lupus-like pathological changes in 8-month mice.

FIG. 22. Protective role of WDFY3 in atherosclerosis in mice and human.

FIGS. 23A-23I. Myeloid deficiency of Wdfy3 exacerbates autoimmune responses in mice with apoptotic cell (AC) infusion. a, Schematics of study design. Mice with LysMCre⁻ mediated myeloid-specific knockout of Wdfy3 (Cret, LysMCre^{+/-}WDFY3^{fl/fl}) and the respective controls (Cre⁻, LysMCre^{-/-}WDFY3^{fl/fl}) were bred, and age- and sex-matched littermates were used for experiments. To increase burden of apoptotic cells (ACs), murine thymocytes underwent UV irradiation to induce apoptosis. Subsequently, ACs were injected intravenously via the tail vein according to the timeline illustrated. Serum samples were collected to determine markers of organ damage and autoantibodies. Kidneys were dissected and sectioned for periodic acid-Schiff (PAS), H&E, and immunofluorescence (IF) staining. b, Serum alanine transaminase (ALT), a marker of liver damage. c, Serum creatinine, a marker of kidney damage. d, Serum anti-nuclear antibodies (ANA). e, Serum anti-dsDNA antibodies. f, PAS staining of kidney sections highlight glomerular basement membrane for the quantification of glomerular size. g, H&E staining of kidney sections were performed to assess mesangial hypercellularity and endocapillary leukocytes. h and i, Immunofluorescence staining of kidney sections using anti-C1q and anti-IgG antibodies was performed. Mean fluorescent intensity (MFI) in glomeruli were quantified. WGA, wheat germ agglutinin. Data are shown as mean±SEM. Each data point represents one biological replicate (mouse).

FIGS. 24A-24N. Myeloid deficiency of Wdfy3 leads to T cell activation through macrophage-intrinsic mechanisms by enhancing antigen presentation and inflammasome activation. a, Schematics of study design. Cre⁻ and Cre⁺ littermates were used to assess T cell activation (b-g), antigen presentation (h and i), and inflammasome activation (j-n). b and c, The percentage of CD44^{high}CD62L^{low} activated T cells in CD4⁺ and CD8⁺ T cells in the blood. d and e, The percentage of CXCR3⁺ activated T cells in CD4⁺ and CD8⁺ T cells in the blood. f and g, The percentage of CD44^{high}CD62L^{low} activated T cells in splenic CD4⁺ and CD8⁺ T cells. h, In vitro antigen presentation assay. BMDMs were pre-treated with synthetic ovalbumin (OVA) for 24 h, followed by incubating with splenic T cells isolated from OT-II or OT-I transgenic mice for 72 h to assess OVA₃₂₃₋₃₃₉ or OVA₂₅₇₋₂₆₄-specific activation of CD4⁺ T cells (OT-II mice) or CD8⁺ T cells (OT-I mice) that recognize OVA peptide residues when presented by the MHC-II or MHC-I molecules, respectively. The OT-II or OT-I mouse T cells were pre-labeled by carboxy-fluorescein succinimidyl ester (CFSE) to monitor the dilution of CFSE signal in daughter cells as T cells become activated and divide. i, In vivo antigen presentation assay. Mice were immunized with OVA₃₂₃₋₃₃₉ for 24 h before the injection of CFSE-labeled splenic CD4⁺ T cells from CD90.1⁺ OT-II mice. 72 h after injection, CFSE dilution in CD4+CD90.1⁺ splenic T cells was assessed. j, Serum IL-18. k, The expression of pro-IL-1b in F4/80⁺ splenic macrophages assessed by flow cytometry. 1, Analysis of intracellular caspase-1 cleavage in F4/80⁺ splenic macrophages by Western blotting. The intensity of the active form of caspase-1, p20, was quantified and compared to the intensity of the pro-caspase-1 (p45). The ratio of p20 intensity to p45 intensity was calculated, and this ratio was normalized to the control group (Cre−), which was assigned a value of 1.0 for standardization. m, BMDMs were co-incubated with ACs at a 5:1 ratio of AC:BMDM for 8 h. Culture media were collected to determine IL-1b by ELISA. n, Human peripheral blood monocytes were differentiated into macrophages (HMDMs) followed by transfection of non-targeting control siRNA (si-Ctrl) or siRNAs targeting WDFY3 (si-WDFY3). At day 7, HMDMs were primed with 20 ng/mL lipopolysaccharide (LPS) for 3 h and then treated with 2 mM ATP for an additional 1 h to induce inflammasome activation. To assess if AC induces caspase-1 cleavage, we co-incubated ACs with HMDMs for 4 h at a 5:1 ratio of AC:HMDM. Unbound ACs were washed away. HMDMs were harvested to determine caspase-1 cleavage by Western blotting. Data are shown as mean±SEM. Each data point represents one biological replicate.

FIGS. 25A-25K. Transgenic overexpression of WDFY3 enhances efferocytosis in vitro in macrophages and in vivo in mice. a, Schematics of experimental design. The hWDFY3Tg mice have the human WDFY3 cDNA inserted into the Rosa26 locus for constitutive overexpression. b, Validation of overexpression of WDFY3 in BMDMs by Western blotting. c, BMDMs were incubated with ACs labeled by Hoechst, which stains DNA and is pH insensitive, and pH rodo, which is pH-sensitive and fluoresces only under an acidified environment in the phagolysosome. The percentage of Hoechst⁺ BMDMs indicates engulfment. The percentage of Hoechst⁺/pH rodo⁺ BMDMs in Hoechst⁺ BMDMs indicates acidification of the engulfed corpses. d, BMDMs were incubated with Hoechst-labeled ACs to allow for efferocytosis. After removal of unbound ACs, BMDMs were collected and treated with digitonin to remove non-membrane bound LC3, and then immunostained for LC3 that is lipidated and membrane bound. LC3-II staining was then quantified by flow cytometry for BMDMs that had engulfed Hoechst-labeled ACs. e, Peritoneal exudates were seeded and nonadherent cells were removed. The remaining peritoneal macrophages (PMs) were cultured for 18 h and then incubated with Hoechst-labeled ACs to allow for efferocytosis. After 1 h, unbound ACs were washed away, and PMs were harvested to quantify the % of Hoechst+ PMs by flow cytometry. f, Schematics of experimental design for in vivo thymus efferocytosis. Mice were injected intraperitoneally with dexamethasone to induce thymocyte apoptosis. 18 h after injection, thymi were dissected for assessment of the following parameters. g, Thymus weight. h, Total number of cells per thymus. i, The percentage of Annexin V⁺ cells per thymus. j. In situ efferocytosis. Thymic sections were stained with TUNEL for ACs, and CD68 for macrophages. The ratio of macrophage associated TUNEL⁺ cells vs. free TUNEL⁺ cells was quantified to assess in situ efferocytosis in the thymus. The white and yellow arrows highlight free and macrophage associated TUNEL⁺ cells, respectively. Lower weight and cellularity, lower percentage of Annexin V+ cells, and higher in situ efferocytosis indicate enhanced efferocytosis capacity. k, In vivo PM efferocytosis assay. 1×10⁷ TAMRA⁺ ACs were injected intraperitoneally, and peritoneal exudates were collected after 15 min. After blocking with CD16/32, peritoneal cells were stained with F4/80 antibody to label PMs and the percentage of TAMRA⁺ PMs was determined by flow cytometry. Data are shown as mean±SEM. Each data point represents one mouse.

FIGS. 26A-26H. Transgenic overexpression of WDFY3 mitigates autoimmune responses in mice with AC infusion. a. Schematics of study design. Age- and sex-matched Ctrl and hWDFY3T8 littermates were used for experiments. To increase burden of ACs, murine thymocytes underwent UV irradiation to induce apoptosis. Subsequently, ACs were injected intravenously via the tail vein according to the timeline illustrated. Serum samples were collected to determine markers of organ damage and autoantibodies. Kidneys were dissected and sectioned for PAS and IF staining. b, Serum ALT. c, Serum creatinine. d, Serum ANA. e, Serum antidsDNA antibodies. f, PAS staining of kidney sections for the quantification of glomerular size. g and h, IF staining of kidney sections using anti-C1q and anti-IgG antibodies. Data are shown as mean±SEM. Each data point represents one biological replicate (mouse).

FIGS. 27A-27N. Transgenic overexpression of WDFY3 suppresses T cell activation partly through macrophage intrinsic mechanisms by suppressing antigen presentation and inflammasome activation. a, Schematics of study design. Ctrl and hWDFY3Tg littermates were used to assess T cell activation (b-g), antigen presentation (h and i), and inflammasome activation (j-m). b and c, The percentage of CD44^{high}CD62L^{low} activated T cells in CD4⁺ and CD8⁺ T cells in the blood. d and e, The percentage of CXCR3⁺ activated T cells in CD4⁺ and CD8⁺ T cells in the blood. f and g, The percentage of CD44^{high}CD62L^{low} activated T cells in splenic CD4⁺ and CD8⁺ T cells. h, Efferocyte-T cell interaction assay. BMDMs were pre-incubated with ACs for 24 h to induced efferocytosis, followed by incubating with CFSE-labeled CD4⁺ splenic T cells for 72 h to assess efferocyte-mediated activation of CD4⁺ T cells. i, In vivo antigen presentation assay. Mice were immunized with OVA₃₂₃₋₃₃₉ for 24 h before the injection of CFSE-labeled splenic CD4⁺ T cells from CD90.1⁺ OT-II mice. 72 h after injection, CFSE dilution in CD4⁺CD90.1⁺ splenic T cells was assessed. j, Serum IL-18 in 15-month-old mice. k, The expression of pro-IL-1b in F4/80⁺ splenic macrophages assessed by flow cytometry. 1, Analysis of intracellular caspase-1 cleavage in F4/80⁺ splenic macrophages by Western blotting. The ratio of p20 intensity to p45 intensity was calculated, and this ratio was normalized to the control group (Ctrl), which was assigned a value of 1.0 for standardization. m, BMDMs were co-incubated with ACs at a 5:1 ratio of AC:BMDM for 8 h. Culture media were collected to determine IL-1b by ELISA. n, Schematic figure to illustrate the role of WDFY3 deficiency vs. overexpression in regulating autoimmunity via efferocytosis-dependent mechanisms as well cell-intrinsic mechanisms independent of efferocytosis. Data are shown as mean±SEM. Each data point represents one biological replicate.

FIGS. 28A-28E. Increasing Alfy levels by introducing the rs17368018 is sufficient to blunt the autoimmune-response related neuroinflammation and organ damage evoked by systemic AC administration. A. Schematic representation of the experiment: Adult wildtype (WT) and Alfy^{Var} mice were systemically administered ACs as previously described. B. Creation of the Alfy^{Var} mice. The location of SNP rs17368018 in the WDFY3 locus, and the orthologous location in Wdfy3 (indicated by ‘*’). WDFY3 and Wdfy3 are the genes that encode Alfy in human and mice, respectively. To create the orthologous SNP in mice, the adenosine was replaced with a guanine. C-D. Quantitative RT-PCR of C. human or D. mouse brain RNA. C. Cortical Broadmann Area 9 (BA9) in individuals genotyped with the rs17368018 variant. Two-tailed student t-test reveals significantly higher levels of WDFY3 expression (p=0.025). WDFY3^{+/+}=patients' wildtype for Alfy; and WDFY3^{rs/+}=carriers of rs17368018. n=14 WDFY3^{+/+}, n=7 WDFY3^{rs/+}. D. Introduction of the A>G variant in mice, reproduces the increased levels of Wdfy3. ANOVA with Fisher PLSD. n=3/genotype. *, p<0.05. WT=mice wildtype for Alfy; Alfy^{Var}=mice carrying the rs17368018 mutation. E. Serum alanine aminotransferase (ALT) levels in mice after system injection of ACs. The presence of elevated Alfy levels protects against organ damage, as indicated by diminished ALT levels. ALT levels were obtained from serum using a HESKA element DC with 120 μL serum delivered on ice and analyzed by the diagnostic lab at the Institute of Comparative Medicine (ICM), Columbia University Irving Medical Center.

FIGS. 29A-29D. Neuropathology in AC-injected animals. Iba1 staining of microglia and GFAP staining of astrocytes reveal a neuroinflammation phenotype in AC-injected mice, that is attenuated upon increased levels of Alfy. A, B. Iba1 staining of A. somatosensory cortex and B. hippocampus. A. AC injected mice show multiple stress-primed microglia that are multinucleated compared to non-injected wildtype mice. The presence of elevated Alfy levels in the Alfy^{Var} mice show an attenuated response. Scale bar=25 μm B. The increased number of Iba1 positive cells visible in AC-injected WT mice was notably attenuated when Alfy levels were elevated. Scale bar=100 μm. C, D. GFAP staining of C. striatum and D. corpus callosum. C. AC injected mice show a strong reactivity including indications of glial scarring (arrow). D. In corpus callosum, reactivity extended beyond the borders into the cortex and striatum. Alfy^{Var} show increased reactivity with respect to non-injected controls, but reactivity was markedly attenuated when compared to AC-injected WT mice. Scale bar=100 μm.

FIGS. S1A-S1D. Accumulation of apoptotic cells (ACs) after intravenously injection. a. Schematics of study design: 2×10⁷ TAMRA-labeled apoptotic murine thymocytes (ACs) were injected intravenously via tail vein injection and mice were euthanized either 24 h or 48 h post-injection. Controls were injected with PBS and mice were euthanized 48 h post-injection. Spleen, liver, and kidney were dissected to determine the % of TAMRA⁺ single cells that indicates AC accumulation by flow cytometry. TAMRA+ cells were mostly accumulated in spleen (b), but not liver (c) or kidney (d). Signals in controls with PBS injection (no ACs) represent autofluorescence. Myeloid deficiency of Wdfy3 exacerbated TAMRA⁺ cell accumulation in the spleen (a). Data are shown as mean±SEM. Each data point represents one mouse. n=3 mice as indicated.

FIGS. S2A-S2K. Myeloid deficiency of Wdfy3 in mice leads to progressive autoimmunity. a, Schematics of study design. Cre⁻ and Cre⁺ mice were euthanized at 52-60-week-old to assess successful knockout and impaired efferocytosis in BMDMs. Organ weight was measured. Serum samples were collected to determine markers of organ damage and autoantibodies. Kidneys were dissected for periodic acid-Schiff (PAS) and immunofluorescence (IF) staining. b, Successful knockout of Wdfy3 at protein level was confirmed by Western blotting. c, BMDMs of Cre⁻ mice showed impaired engulfment and lysosomal acidification of the engulfed ACs as determined by flow cytometry. d, Organ weight was measured. e, Serum alanine transaminase (ALT). f, Serum creatinine. g, Serum anti-nuclear antibodies (ANA). h, Serum anti-dsDNA antibodies. i, PAS staining of kidney sections for the quantification of glomerular size. j and k, IF staining of kidney sections using anti-C1q and anti-IgG antibodies was performed. Mean fluorescent intensity (MFI) of C1q and IgG in glomeruli were quantified. WGA, wheat germ agglutinin. Data are shown as mean±SEM. Each data point represents a biological replicate (mouse). n=4-9 mice per group as indicated.

FIGS. S3A-S3J. Myeloid deficiency of Wdfy3 did not affect autoimmune phenotypes in young mice. a, Schematics of study design. Cre⁻ and Cre⁺ mice were euthanized at 14-16-week-old. Serum samples were collected to determine markers of organ damage and autoantibodies. Kidneys were dissected and sectioned for PAS, H&E, and IF staining. b, Organ weight was measured. c, Serum ALT, a marker of liver damage. d, Serum creatinine, a marker of kidney damage. e, Serum ANA. f, Serum anti-dsDNA antibodies. g, PAS staining of kidney sections highlight glomerular basement membrane for the quantification of glomerular size. h, H&E staining of kidney sections were performed to assess mesangial hypercellularity and endocapillary leukocytes. i and j, Immunofluorescence staining of kidney sections using anti-C1q and anti-IgG antibodies was performed. Mean fluorescent intensity (MFI) in glomeruli were quantified. WGA, wheat germ agglutinin. Data are shown as mean±SEM. Each data point represents one biological replicate (mouse).

FIGS. S4A-S4Y. Serum cytokine and chemokine profiling. Serum from Cre⁻ or Cre⁺ mice at 8-weekold were collected and assessed using a 26-plex ProCartaPlex mouse cytokine and chemokine panel. Data are shown as mean±SEM. Each data point represents one mouse. n=7-11 mice as indicated.

FIGS. S5A-S5I. Transgenic overexpression of WDFY3 mitigates progressive autoimmunity in middle-aged mice. a, Schematics of study design. Age- and sex-matched Ctrl and hWDFY3Tg mice were euthanized at 52-60-week-old. Efferocytosis of BMDMs was determined. c, Organ weight was measured. Serum samples were collected to determine markers of organ damage and autoantibodies. Kidneys were dissected and sectioned for PAS and IF staining. b, Efferocytosis was enhanced in BMDMs of hWDFY3^{Tg} mice. c, Organ weight, d, Serum ALT. e, Serum creatinine. f, Serum ANA. g, Serum anti-dsDNA antibodies. h, PAS staining of kidney sections for the quantification of glomerular size. i, IF staining of kidney sections using anti-IgG antibodies. Data are shown as mean±SEM. Each data point represents a biological replicate (mouse). n=6-13 mice per group as indicated.

FIGS. S6A-S6B. Omics approaches to determine the effects of WDFY3 on the transcriptomic signature of BMDMs and the proximal proteins of WDFY3. a, The effects of transgenic overexpression of WDFY3 on the transcriptomic signature of BMDMs were determined by RNA-seq. Volcano plot shows very few differentially expressed (DE) genes in hWDFY3Tg vs. Ctrl BMDMs (n=5 mice), suggesting the lack of effects by WDFY3 overexpression at the transcriptomic level. b, Proximity labeling by TurboID identifies proximal proteins to C-WDFY3. The schematics of TurboID, an engineered biotin ligase that uses ATP to convert biotin into biotin-AMP, a reactive intermediate that covalently labels proximal proteins (˜20 nm zone). Labeled biotinylated proteins are pulled down for mass spectrometry. (B) Lenti-TurboID-WDFY3₂₅₄₃₋₃₅₂₆ construct was transduced to BMDMs for biotin labeling of proximal proteins of WDFY3 in BMDMs with or without AC engulfment. The labeling starts by adding biotin and ATP. BMDMs were then lysed and biotinylated proteins pulled down by streptavidin-conjugated beads. Biotinylated proteins were analyzed by mass spectrometry. Data were analyzed and visualized by Perseus software using ANOVA followed by Tukey post hoc test to nominate the top hits as shown, including proteins in the Arp2/3 complex (red), proteins regulating F-actin dynamics (purple), and myosins (green). BMDMs were treated with T: TurboID transduction; TBA: TurboID transduction followed by AC and biotin incubation; TB: TurboID transduction followed by biotin incubation; B: Biotin only; AB: AC and biotin incubation only. n=3 mice.

FIGS. S7A-S7B. WDFY3 and WDFY4 expression across tissue and cell types based on data in the Human Protein Atlas resource. Single cell transcriptomics data for 25 tissues and peripheral blood mononuclear cells (PBMCs) were analyzed. These datasets were respectively retrieved from the Single Cell Expression Atlas, the Human Cell Atlas, the Gene Expression Omnibus, the Allen Brain Map, and the European Genome-phenome Archive. The panels are screenshots from the Human Protein Atlas resource. a, WDFY3 is the most abundantly expressed in neuronal cells. Among immune cells, WDFY3 is the most abundantly expressed in monocytes and macrophages, with absent expression in other immune cells, such as B cells and dendritic cells. b, WDFY4, a paralog of WDFY3, is the most abundantly expressed in B cells and dendritic cells among immune cells.

### DETAILED DESCRIPTION

Phagocytic clearance of dying cells, termed efferocytosis, is essential for maintaining tissue homeostasis, yet the understanding of efferocytosis regulation remains incomplete. A FACS-based, genome-wide CRISPR knockout screen was performed in primary mouse macrophages to search for novel regulators of efferocytosis. The results show that Wdfy3 knockout in macrophages specifically impairs uptake, but not binding, of apoptotic cells due to defective actin disassembly. Additionally, WDFY3 interacts with GABARAP, thus facilitating LC3 lipidation and subsequent lysosomal acidification to permit the degradation of apoptotic cell components. Mechanistically, while the C-terminus of WDFY3 is sufficient to rescue the impaired degradation induced by Wdfy3 knockout, full-length WDFY3 is required to reconstitute the uptake of apoptotic cells. Finally, WDFY3 is also important for efficient efferocytosis in vivo in mice and in vitro in primary human macrophages.

### Definitions

A “vector” refers to a macromolecule or association of macromolecules that comprises or associates with a polynucleotide, and which can be used to mediate delivery of the polynucleotide to a cell, either in vitro or in vivo. Illustrative vectors include, for example, plasmids, viral vectors, liposomes and other gene delivery vehicles. The polynucleotide to be delivered, sometimes referred to as a “target polynucleotide” or “transgene,” may comprise a coding sequence of interest in gene therapy (such as a gene encoding a protein of therapeutic interest), a coding sequence of interest in vaccine development (such as a polynucleotide expressing a protein, polypeptide or peptide suitable for eliciting an immune response in a mammal), and/or a selectable or detectable marker.

“Transduction,” “transfection,” “transformation” or “transducing” as used herein, are terms referring to a process for the introduction of an exogenous polynucleotide into a host cell leading to expression of the polynucleotide, e.g., the transgene in the cell, and includes the use of recombinant virus to introduce the exogenous polynucleotide to the host cell. Transduction, transfection or transformation of a polynucleotide in a cell may be determined by methods well known to the art including, but not limited to, protein expression (including steady state levels), e.g., by ELISA, flow cytometry and Western blot, measurement of DNA and RNA by hybridization assays, e.g., Northern blots, Southern blots and gel shift mobility assays. Methods used for the introduction of the exogenous polynucleotide include well-known techniques such as viral infection or transfection, lipofection, transformation and electroporation, as well as other non-viral gene delivery techniques. The introduced polynucleotide may be stably or transiently maintained in the host cell.

“Gene delivery” refers to the introduction of an exogenous polynucleotide into a cell for gene transfer, and may encompass targeting, binding, uptake, transport, localization, replicon integration and expression.

“Gene transfer” refers to the introduction of an exogenous polynucleotide into a cell which may encompass targeting, binding, uptake, transport, localization and replicon integration, but is distinct from and does not imply subsequent expression of the gene.

“Gene expression” or “expression” refers to the process of gene transcription, translation, and post-translational modification.

An “infectious” virus or viral particle is one that comprises a polynucleotide component which it is capable of delivering into a cell for which the viral species is trophic. The term does not necessarily imply any replication capacity of the virus.

The term “polynucleotide” refers to a polymeric form of nucleotides of any length, including deoxyribonucleotides or ribonucleotides, or analogs thereof. A polynucleotide may comprise modified nucleotides, such as methylated or capped nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment described herein that is a polynucleotide encompasses both the double-stranded form and each of two complementary single-stranded forms known or predicted to make up the double-stranded form.

An “isolated” polynucleotide, e.g., plasmid, virus, polypeptide or other substance refers to a preparation of the substance devoid of at least some of the other components that may also be present where the substance or a similar substance naturally occurs or is initially prepared from. Thus, for example, an isolated substance may be prepared by using a purification technique to enrich it from a source mixture. Isolated nucleic acid, peptide or polypeptide is present in a form or setting that is different from that in which it is found in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. The isolated nucleic acid molecule may be present in single-stranded or double-stranded form. When an isolated nucleic acid molecule is to be utilized to express a protein, the molecule will contain at a minimum the sense or coding strand (i.e., the molecule may single-stranded), but may contain both the sense and anti-sense strands (i.e., the molecule may be double-stranded). Enrichment can be measured on an absolute basis, such as weight per volume of solution, or it can be measured in relation to a second, potentially interfering substance present in the source mixture. Increasing enrichments of the embodiments of this disclosure are envisioned. Thus, for example, a 2-fold enrichment, 10-fold enrichment, 100-fold enrichment, or a 1000-fold enrichment.

A “transcriptional regulatory sequence” refers to a genomic region that controls the transcription of a gene or coding sequence to which it is operably linked. Transcriptional regulatory sequences of use in the present disclosure generally include at least one transcriptional promoter and may also include one or more enhancers and/or terminators of transcription.

“Operably linked” refers to an arrangement of two or more components, wherein the components so described are in a relationship permitting them to function in a coordinated manner. By way of illustration, a transcriptional regulatory sequence or a promoter is operably linked to a coding sequence if the TRS or promoter promotes transcription of the coding sequence. An operably linked TRS is generally joined in cis with the coding sequence, but it is not necessarily directly adjacent to it.

“Heterologous” means derived from a genotypically distinct entity from the entity to which it is compared. For example, a polynucleotide introduced by genetic engineering techniques into a different cell type is a heterologous polynucleotide (and, when expressed, can encode a heterologous polypeptide). Similarly, a transcriptional regulatory element such as a promoter that is removed from its native coding sequence and operably linked to a different coding sequence is a heterologous transcriptional regulatory element.

A “terminator” refers to a polynucleotide sequence that tends to diminish or prevent read-through transcription (i.e., it diminishes or prevent transcription originating on one side of the terminator from continuing through to the other side of the terminator). The degree to which transcription is disrupted is typically a function of the base sequence and/or the length of the terminator sequence. In particular, as is well known in numerous molecular biological systems, particular DNA sequences, generally referred to as “transcriptional termination sequences” are specific sequences that tend to disrupt read-through transcription by RNA polymerase, presumably by causing the RNA polymerase molecule to stop and/or disengage from the DNA being transcribed. Typical examples of such sequence-specific terminators include polyadenylation (“polyA”) sequences, e.g., SV40 polyA. In addition to or in place of such sequence-specific terminators, insertions of relatively long DNA sequences between a promoter and a coding region also tend to disrupt transcription of the coding region, generally in proportion to the length of the intervening sequence. This effect presumably arises because there is always some tendency for an RNA polymerase molecule to become disengaged from the DNA being transcribed, and increasing the length of the sequence to be traversed before reaching the coding region would generally increase the likelihood that disengagement would occur before transcription of the coding region was completed or possibly even initiated. Terminators may thus prevent transcription from only one direction (“uni-directional” terminators) or from both directions (“bi-directional” terminators), and may be comprised of sequence-specific termination sequences or sequence-non-specific terminators or both. A variety of such terminator sequences are known in the art; and illustrative uses of such sequences within the context of the present disclosure are provided below.

“Host cells,” “cell lines,” “cell cultures,” “packaging cell line” and other such terms denote higher eukaryotic cells, such as mammalian cells including human cells, useful in the present disclosure, e.g., to produce recombinant virus or recombinant fusion polypeptide. These cells include the progeny of the original cell that was transduced. It is understood that the progeny of a single cell may not necessarily be completely identical (in morphology or in genomic complement) to the original parent cell.

“Recombinant,” as applied to a polynucleotide means that the polynucleotide is the product of various combinations of cloning, restriction and/or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter.

An “expression vector” is a vector comprising a region which encodes a gene product of interest and is used for effecting the expression of the gene product in an intended target cell. An expression vector also comprises control elements operatively linked to the encoding region to facilitate expression of the protein in the target. The combination of control elements and a gene or genes to which they are operably linked for expression is sometimes referred to as an “expression cassette,” a large number of which are known and available in the art or can be readily constructed from components that are available in the art.

The terms “polypeptide” and “protein” are used interchangeably herein to refer to polymers of amino acids of any length. The terms also encompass an amino acid polymer that has been modified; for example, disulfide bond formation, glycosylation, acetylation, phosphorylation, lipidation, or conjugation with a labeling component.

The term “exogenous,” when used in relation to a protein, gene, nucleic acid, or polynucleotide in a cell or organism refers to a protein, gene, nucleic acid, or polynucleotide which has been introduced into the cell or organism by artificial or natural means. An exogenous nucleic acid may be from a different organism or cell, or it may be one or more additional copies of a nucleic acid which occurs naturally within the organism or cell. By way of a non-limiting example, an exogenous nucleic acid is in a chromosomal location different from that of natural cells or is otherwise flanked by a different nucleic acid sequence than that found in nature, e.g., an expression cassette which links a promoter from one gene to an open reading frame for a gene product from a different gene.

“Transformed” or “transgenic” is used herein to include any host cell or cell line, which has been altered or augmented by the presence of at least one recombinant DNA sequence. The host cells of the present disclosure are typically produced by transfection with a DNA sequence in a plasmid expression vector, as an isolated linear DNA sequence, or infection with a recombinant viral vector.

The term “sequence homology” means the proportion of base matches between two nucleic acid sequences or the proportion amino acid matches between two amino acid sequences. When sequence homology is expressed as a percentage, e.g., 50%, the percentage denotes the proportion of matches over the length of a selected sequence that is compared to some other sequence. Gaps (in either of the two sequences) are permitted to maximize matching; gap lengths of 15 bases or less are usually used, 6 bases or less e.g., with 2 bases or less. When using oligonucleotides as probes or treatments, the sequence homology between the target nucleic acid and the oligonucleotide sequence is generally not less than 17 target base matches out of 20 possible oligonucleotide base pair matches (85%); not less than 9 matches out of 10 possible base pair matches (90%), or not less than 19 matches out of 20 possible base pair matches (95%).

Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less or with 2 or less. Alternatively, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. The two sequences or parts thereof are more homologous if their amino acids are greater than or equal to 50% identical when optimally aligned using the ALIGN program.

The term “corresponds to” is used herein to mean that a polynucleotide sequence is structurally related to all or a portion of a reference polynucleotide sequence, or that a polypeptide sequence is structurally related to all or a portion of a reference polypeptide sequence, e.g., they have at least 80%, 85%, 90%, 95% or more, e.g., 99% or 100%, sequence identity. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The term “sequence identity” means that two polynucleotide sequences are identical (i.e., on a nucleotide-by-nucleotide basis) over the window of comparison. The term “percentage of sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denote a characteristic of a polynucleotide sequence, wherein the polynucleotide comprises a sequence that has at least 85 percent sequence identity, e.g., at least 90 to 95 percent sequence identity, or at least 99 percent sequence identity as compared to a reference sequence over a comparison window of at least 20 nucleotide positions, frequently over a window of at least 20-50 nucleotides, wherein the percentage of sequence identity is calculated by comparing the reference sequence to the polynucleotide sequence which may include deletions or additions which total 20 percent or less of the reference sequence over the window of comparison.

“Conservative” amino acid substitutions are, for example, aspartic-glutamic as polar acidic amino acids; lysine/arginine/histidine as polar basic amino acids; leucine/isoleucine/methionine/valine/alanine/glycine/proline as non-polar or hydrophobic amino acids; serine/threonine as polar or uncharged hydrophilic amino acids. Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting polypeptide. Whether an amino acid change results in a functional polypeptide can readily be determined by assaying the specific activity of the polypeptide. Naturally occurring residues are divided into groups based on common side-chain properties: (1) hydrophobic: norleucine, met, ala, val, leu, ile; (2) neutral hydrophilic: cys, ser, thr; (3) acidic: asp, glu; (4) basic: asn, gln, his, lys, arg; (5) residues that influence chain orientation: gly, pro; and (6) aromatic; trp, tyr, phe.

The disclosure also envisions polypeptides with non-conservative substitutions. Non-conservative substitutions entail exchanging a member of one of the classes described above for another.

As used herein, “individual” (as in the subject of the treatment) means a mammal. Mammals include, for example, humans; non-human primates, e.g., apes and monkeys; and non-primates, e.g., dogs, cats, rats, mice, cattle, horses, sheep, and goats. Non-mammals include, for example, fish and birds.

The term “disease” or “disorder” are used interchangeably and are used to refer to neurodegenerative or proteinopathy diseases or conditions.

“Substantially” as the term is used herein means completely or almost completely; for example, a composition that is “substantially free” of a component either has none of the component or contains such a trace amount that any relevant functional property of the composition is unaffected by the presence of the trace amount, or a compound is “substantially pure” is there are only negligible traces of impurities present.

“Treating” or “treatment” within the meaning herein refers to an alleviation of symptoms associated with a disorder or disease, “inhibiting” means inhibition of further progression or worsening of the symptoms associated with the disorder or disease, and “preventing” refers to prevention of the symptoms associated with the disorder or disease.

As used herein, an “effective amount” or a “therapeutically effective amount” of an agent, e.g., a recombinant AAV encoding a gene product, refers to an amount of the agent that alleviates, in whole or in part, symptoms associated with the disorder or condition, or halts or slows further progression or worsening of those symptoms, or prevents or provides prophylaxis for the disorder or condition, e.g., an amount that is effective to prevent, inhibit or treat in the individual one or more symptoms.

In particular, a “therapeutically effective amount” refers to an amount effective, at dosages and for periods of time necessary, to achieve the desired therapeutic result. A therapeutically effective amount is also one in which any toxic or detrimental effects of the agent(s) are outweighed by the therapeutically beneficial effects.

### Exemplary WDFY3/Alfy Nucleic Acid and Amino Acid Sequences

In one embodiment, WDFY3/Alfy comprises a polypeptide having the sequence in NCBI Reference Sequence: NM_014991.6, e.g.,

(SEQ ID NO: 1)
MNMVKRIMGRPRQEECSPQDNALGLMHLRRLFTELCHPPRHMTQ
KEQEEKLYMMLPVFNRVFGNAPPNTMTEKFSDLLQFTTQVSRLMVTEIRRRASNKSTE
AASRAIVQFLEINQSEEASRGWMLLTTINLLASSGQKTVDCMTTMSVPSTLVKCLYLF
FDLPHVPEAVGGAQNELPLAERRGLLQKVFVQILVKLCSFVSPAEELAQKDDLQLLES
AITSWCPPYNLPWRKSAGEVLMTISRHGLSVNVVKYIHEKECLSTCVQNMQQSDDLSP
LEIVEMFAGLSCFLKDSSDVSQTLLDDFRIWQGYNFLCDLLLRLEQAKEAESKDALKD
LVNLITSLTTYGVSELKPAGITTGAPFLLPGFAVPQPAGKGHSVRNVQAFAVLQNAFL
KAKTSFLAQIILDAITNIYMADNANYFILESQHTLSQFAEKISKLPEVQNKYFEMLEF
VVFSLNYIPCKELISVSILLKSSSSYHCSIIAMKTLLKFTRHDYIFKDVFREVGLLEV
MVNLLHKYAALLKDPTQALNEQGDSRNNSSVEDQKHLALLVMETLTVLLQGSNTNAGI
FREFGGARCAHNIVKYPQCRQHALMTIQQLVLSPNGDDDMGTLLGLMHSAPPTELQLK
TDILRALLSVLRESHRSRTVFRKVGGFVYITSLLVAMERSLSCPPKNGWEKVNQNQVE
ELLHTVFCTLTAAMRYEPANSHFFKTEIQYEKLADAVRFLGCFSDLRKISAMNVEPSN
TQPFQRLLEEDVISIESVSPTLRHCSKLFIYLYKVATDSFDSRAEQIPPCLTSESSLP
SPWGTPALSRKRHAYHSVSTPPVYPPKNVADLKLHVTTSSLQSSDAVIIHPGAMLAML
DLLASVGSVTQPEHALDLQLAVANILQSLVHTERNQQVMCEAGLHARLLQRCSAALAD
EDHSLHPPLQRMFERLASQALEPMVLREFLRLASPLNCGAWDKKLLKQYRVHKPSSLS
YEPEMRSSMITSLEGLGTDNVFSLHEDNHYRISKSLVKSAEGSTVPLTRVKCLVSMTT
PHDIRLHGSSVTPAFVEFDTSLEGFGCLFLPSLAPHNAPTNNTVTTGLIDGAVVSGIG
SGERFFPPPSGLSYSSWFCIEHFSSPPNNHPVRLLTVVRRANSSEQHYVCLAIVLSAK
DRSLIVSTKEELLQNYVDDFSEESSFYEILPCCARFRCGELIIEGQWHHLVLVMSKGM
LKNSTAALYIDGQLVNTVKLHYVHSTPGGSGSANPPVVSTVYAYIGTPPAQRQIASLV
WRLGPTHFLEEVLPSSNVTTIYELGPNYVGSFQAVCMPCKDAKSEGVVPSPVSLVPEE
KVSFGLYALSVSSLTVARIRKVYNKLDSKAIAKQLGISSHENATPVKLIHNSAGHLNG
SARTIGAALIGYLGVRTFVPKPVATTLQYVGGAAAILGLVAMASDVEGLYAAVKALVC
VVKSNPLASKEMERIKGYQLLAMLLKKKRSLLNSHILHLTFSLVGTVDSGHETSIIPN
STAFQDLLCDFEVWLHAPYELHLSLFEHFIELLTESSEASKNAKLMREFQLIPKLLLT
LRDMSLSQPTIAAISNVLSFLLQGFPSSNDLLRFGQFISSTLPTFAVCEKFVVMEINN
EEKLDTGTEEEFGGLVSANLILLRNRLLDILLKLIYTSKEKTSINLQACEELVKTLGF
DWIMMFMEEHLHSTTVTAAMRILVVLLSNQSILIKFKEGLSGGGWLEQTDSVLINKIG
TVLGFNVGRSAGGRSTVREINRDACHFPGFPVLQSFLPKHTNVPALYFLLMALFLQQP
VSELPENLQVSVPVISCRSKQGCQFDLDSIWTFIFGVPASSGTVVSSIHNVCTEAVEL
LLGMLRSMLTSPWQSEEEGSWLREYPVTLMQFFRYLYHNVPDLASMWMSPDFLCALAA
TVFPFNIRPYSEMVTDLDDEVGSPAEEFKAFAADTGMNRSQSEYCNVGTKTYLTNHPA
KKFVFDFMRVLIIDNLCLTPASKQTPLIDLLLEASPERSTRTQQKEFQTYILDSVMDH
LLAADVLLGEDASLPITSGGSYQVLVNNVFYFTQRVVDKLWQGMENKESKLLIDFIIQ
LIAQSKRRSQGLSLDAVYHCLNRTILYQFSRAHKTVPQQVALLDSLRVLTVNRNLILG
PGNHDQEFISCLAHCLINLHVGSNVDGFGLEAEARMTTWHIMIPSDIEPDGSYSQDIS
EGRQLLIKAVNRVWTELIHSKKQVLEELFKVTLPVNERGHVDIATARPLIEEAALKCW
QNHLAHEKKCISRGEALAPTTQSKLSRVSSGFGLSKLTGSRRNRKESGLNKHSLSTQE
ISQWMFTHIAVVRDLVDTQYKEYQERQQNALKYVTEEWCQIECELLRERGLWGPPIGS
HLDKWMLEMTEGPCRMRKKMVRNDMFYNHYPYVPETEQETNVASEIPSKQPETPDDIP
QKKPARYRRAVSYDSKEYYMRLASGNPAIVQDAIVESSEGEAAQQEPEHGEDTIAKVK
GLVKPPLKRSRSAPDGGDEENQEQLQDQIAEGSSIEEEEKTDNATLLRLLEEGEKIQH
MYRCARVQGLDTSEGLLLFGKEHFYVIDGFTMTATREIRDIETLPPNMHEPIIPRGAR
QGPSQLKRTCSIFAYEDIKEVHKRRYLLQPIAVEVFSGDGRNYLLAFQKGIRNKVYQR
FLAVVPSLTDSSESVSGQRPNTSVEQGSGLLSTLVGEKSVTQRWERGEISNFQYLMHL
NTLAGRSYNDLMQYPVFPWILADYDSEEVDLTNPKTFRNLAKPMGAQTDERLAQYKKR
YKDWEDPNGETPAYHYGTHYSSAMIVASYLVRMEPFTQIFLRLQGGHFDLADRMFHSV
REAWYSASKHNMADVKELIPEFFYLPEFLENSNNFDLGCKQNGTKLGDVILPPWAKGD
PREFIRVHREALECDYVSAHLHEWIDLIFGYKQQGPAAVEAVNVFHHLFYEGQVDIYN
INDPLKETATIGFINNFGQIPKQLFKKPHPPKRVRSRLNGDNAGISVLPGSTSDKIFF
HHLDNLRPSLTPVKELKEPVGQIVCTDKGILAVEQNKVLIPPTWNKTFAWGYADLSCR
LGTYESDKAMTVYECLSEWGQILCAICPNPKLVITGGTSTVVCVWEMGTSKEKAKTVT
LKQALLGHTDTVTCATASLAYHIIVSGSRDRTCIIWDLNKLSFLTQLRGHRAPVSALC
INELTGDIVSCAGTYIHVWSINGNPIVSVNTFTGRSQQIICCCMSEMNEWDTQNVIVT
GHSDGVVRFWRMEFLQVPETPAPEPAEVLEMQEDCPEAQIGQEAQDEDSSDSEADEQS
ISQDPKDTPSQPSSTSHRPRAASCRATAAWCTDSGSDDSRRWSDQLSLDEKDGFIFVN
YSEGQTRAHLQGPLSHPHPNPIEVRNYSRLKPGYRWERQLVERSKLTMHTAFDRKDNA
HPAEVTALGISKDHSRILVGDSRGRVFSWSVSDQPGRSAADHWVKDEGGDSCSGCSVR
FSLTERRHHCRNCGQLFCQKCSRFQSEIKRLKISSPVRVCQNCYYNLQHERGSEDGPR
NC,

### or NCBI Reference Sequence: NP_055806.2, e.g.,

(SEQ ID NO: 2)
MNMVKRIMGR PRQEECSPQD NALGLMHLRR LFTELCHPPR HMTQKEQEEK LYMMLPVFNR
VFGNAPPNTM TEKFSDLLQF TTQVSRLMVT EIRRRASNKS TEAASRAIVQ FLEINQSEEA
SRGWMLLTTI NLLASSGQKT VDCMTTMSVP STLVKCLYLF FDLPHVPEAV GGAQNELPLA
ERRGLLQKVF VQILVKLCSF VSPAEELAQK DDLQLLFSAI TSWCPPYNLP WRKSAGEVLM
TISRHGLSVN VVKYIHEKEC LSTCVQNMQQ SDDLSPLEIV EMFAGLSCFL KDSSDVSQTL
LDDFRIWQGY NFLCDLLLRL EQAKEAESKD ALKDLVNLIT SLTTYGVSEL KPAGITTGAP
FLLPGFAVPQ PAGKGHSVRN VQAFAVLQNA FLKAKTSFLA QIILDAITNI YMADNANYFI
LESQHTLSQF AEKISKLPEV QNKYFEMLEF VVFSLNYIPC KELISVSILL KSSSSYHCSI
IAMKTLLKFT RHDYIFKDVF REVGLLEVMV NLLHKYAALL KDPTQALNEQ GDSRNNSSVE
DQKHLALLVM ETLTVLLQGS NTNAGIFREF GGARCAHNIV KYPQCRQHAL MTIQQLVLSP
NGDDDMGTLL GLMHSAPPTE LQLKTDILRA LLSVLRESHR SRTVFRKVGG FVYITSLLVA
MERSLSCPPK NGWEKVNQNQ VFELLHTVFC TLTAAMRYEP ANSHFFKTEI QYEKLADAVR
FLGCFSDLRK ISAMNVFPSN TQPFQRLLEE DVISIESVSP TLRHCSKLFI YLYKVATDSF
DSRAEQIPPC LTSESSLPSP WGTPALSRKR HAYHSVSTPP VYPPKNVADL KLHVTTSSLQ
SSDAVIIHPG AMLAMLDLLA SVGSVTQPEH ALDLQLAVAN ILQSLVHTER NQQVMCEAGL
HARLLQRCSA ALADEDHSLH PPLQRMFERL ASQALEPMVL REFLRLASPL NCGAWDKKLL
KQYRVHKPSS LSYEPEMRSS MITSLEGLGT DNVFSLHEDN HYRISKSLVK SAEGSTVPLT
RVKCLVSMTT PHDIRLHGSS VTPAFVEFDT SLEGFGCLFL PSLAPHNAPT NNTVTTGLID
GAVVSGIGSG ERFFPPPSGL SYSSWFCIEH FSSPPNNHPV RLLTVVRRAN SSEQHYVCLA
IVLSAKDRSL IVSTKEELLQ NYVDDESEES SFYEILPCCA RFRCGELIIE GQWHHLVLVM
SKGMLKNSTA ALYIDGQLVN TVKLHYVHST PGGSGSANPP VVSTVYAYIG TPPAQRQIAS
LVWRLGPTHF LEEVLPSSNV TTIYELGPNY VGSFQAVCMP CKDAKSEGVV PSPVSLVPEE
KVSFGLYALS VSSLTVARIR KVYNKLDSKA IAKQLGISSH ENATPVKLIH NSAGHLNGSA
RTIGAALIGY LGVRTFVPKP VATTLQYVGG AAAILGLVAM ASDVEGLYAA VKALVCVVKS
NPLASKEMER IKGYQLLAML LKKKRSLLNS HILHLTFSLV GTVDSGHETS IIPNSTAFQD
LLCDFEVWLH APYELHLSLF EHFIELLTES SEASKNAKLM REFQLIPKLL LTLRDMSLSQ
PTIAAISNVL SFLLQGFPSS NDLLRFGQFI SSTLPTFAVC EKFVVMEINN EEKLDTGTEE
EFGGLVSANL ILLRNRLLDI LLKLIYTSKE KTSINLQACE ELVKTLGEDW IMMFMEEHLH
STTVTAAMRI LVVLLSNQSI LIKFKEGLSG GGWLEQTDSV LINKIGTVLG FNVGRSAGGR
STVREINRDA CHFPGFPVLQ SFLPKHTNVP ALYFLLMALF LQQPVSELPE NLQVSVPVIS
CRSKQGCQFD LDSIWTFIFG VPASSGTVVS SIHNVCTEAV FLLLGMLRSM LTSPWQSEEE
GSWLREYPVT LMQFFRYLYH NVPDLASMWM SPDFLCALAA TVFPFNIRPY SEMVTDLDDE
VGSPAEEFKA FAADTGMNRS QSEYCNVGTK TYLTNHPAKK FVFDEMRVLI IDNLCLTPAS
KQTPLIDLLL EASPERSTRT QQKEFQTYIL DSVMDHLLAA DVLLGEDASL PITSGGSYQV
LVNNVFYFTQ RVVDKLWQGM FNKESKLLID FIIQLIAQSK RRSQGLSLDA VYHCLNRTIL
YQFSRAHKTV PQQVALLDSL RVLTVNRNLI LGPGNHDQEF ISCLAHCLIN LHVGSNVDGF
GLEAEARMTT WHIMIPSDIE PDGSYSQDIS EGRQLLIKAV NRVWTELIHS KKQVLEELFK
VTLPVNERGH VDIATARPLI EEAALKCWQN HLAHEKKCIS RGEALAPTTQ SKLSRVSSGF
GLSKLTGSRR NRKESGLNKH SLSTQEISQW MFTHIAVVRD LVDTQYKEYQ ERQQNALKYV
TEEWCQIECE LLRERGLWGP PIGSHLDKWM LEMTEGPCRM RKKMVRNDMF YNHYPYVPET
EQETNVASEI PSKQPETPDD IPQKKPARYR RAVSYDSKEY YMRLASGNPA IVQDAIVESS
EGEAAQQEPE HGEDTIAKVK GLVKPPLKRS RSAPDGGDEE NQEQLQDQIA EGSSIEEEEK
TDNATLLRLL EEGEKIQHMY RCARVQGLDT SEGLLLFGKE HFYVIDGFTM TATREIRDIE
TLPPNMHEPI IPRGARQGPS QLKRTCSIFA YEDIKEVHKR RYLLQPIAVE VFSGDGRNYL
LAFQKGIRNK VYQRFLAVVP SLTDSSESVS GQRPNTSVEQ GSGLLSTLVG EKSVTQRWER
GEISNFQYLM HLNTLAGRSY NDLMQYPVFP WILADYDSEE VDLTNPKTFR NLAKPMGAQT
DERLAQYKKR YKDWEDPNGE TPAYHYGTHY SSAMIVASYL VRMEPFTQIF LRLQGGHEDL
ADRMFHSVRE AWYSASKHNM ADVKELIPEF FYLPEFLENS NNFDLGCKQN GTKLGDVILP
PWAKGDPREF IRVHREALEC DYVSAHLHEW IDLIFGYKQQ GPAAVEAVNV FHHLFYEGQV
DIYNINDPLK ETATIGFINN FGQIPKQLFK KPHPPKRVRS RINGDNAGIS VLPGSTSDKI
FFHHLDNLRP SLTPVKELKE PVGQIVCTDK GILAVEQNKV LIPPTWNKTF AWGYADLSCR
LGTYESDKAM TVYECLSEWG QILCAICPNP KLVITGGTST VVCVWEMGTS KEKAKTVTLK
QALLGHTDTV TCATASLAYH IIVSGSRDRT CIIWDLNKLS FLTQLRGHRA PVSALCINEL
TGDIVSCAGT YIHVWSINGN PIVSVNTFTG RSQQIICCCM SEMNEWDTQN VIVTGHSDGV
VRFWRMEFLQ VPETPAPEPA EVLEMQEDCP EAQIGQEAQD EDSSDSEADE QSISQDPKDT
PSQPSSTSHR PRAASCRATA AWCTDSGSDD SRRWSDQLSL DEKDGFIFVN YSEGQTRAHL
QGPLSHPHPN PIEVRNYSRL KPGYRWERQL VERSKLTMHT AFDRKDNAHP AEVTALGISK
DHSRILVGDS RGRVFSWSVS DQPGRSAADH WVKDEGGDSC SGCSVRESLT ERRHHCRNCG
QLFCQKCSRF QSEIKRLKIS SPVRVCQNCY YNLQHERGSE DGPRNC,

as well as a polypeptide with at least 80%, 85%, 90%, 95% or more, e.g., 99% or more, amino acid sequence identity thereto, or a portion thereof with the activity of SEQ ID NO: 1 or SEQ ID NO: 2.

An exemplary mRNA sequence for WDFY3/Alfy comprises:

(SEQ ID NO: 9)
agacagccag cgggaggtgg agaaagcagg aggaggagga ggattaaaga tggccaccaa
cagctgcggg aaacggcaac aacccctcac tttccgggat ggtccctgcg ggtcggcccg
gccttgatgg agagaagaaa cccgaggagc gccgaggctg aggcggcggc ggcggggacc
cagcgaggac gaggacgcgg cggagcaggg acgggggcag gagaagggaa aggcggcggc
gtcgctgccc ctgctgccta gcaccgctgc ctggcccggc ggaccggttc ccatacctcg
cggccgcaga atcgagctcg ggccccggcc cccggcccgc ggcgcggggc tcccgggccc
cgccgcggac gtcgcgccgg tcgccccttc cccgtagccc gtgcgccctc ggcgcggagc
cccggcccgc cgcggtcccg tctcctgggc ctgtcccgcc cgcgccctcc gccggccctc
aggtataata cttctccacg tctgcttcag gaagaaagtg cctgccattc ttatcatttc
taagcaggtt catgccagcc cagaacagag aatcagctgg agcccagatt tcaagttttg
agtaaaatac cttcaagcga atgggcccta ttgtgctcac acattcagaa cctgttaccc
aaggaattcc ctaaagaatt agaagtgcgt ctcaccaacc agccaagatg aacatggtga
agaggatcat ggggcggccg aggcaggagg agtgcagccc acaagacaac gccttaggac
tgatgcacct ccgccggctc ttcacggagt tgtgccatcc tccccggcac atgactcaga
aggaacaaga agagaaactg tatatgatgc tgccagtgtt taacagggtt tttggaaatg
ctccgccgaa tacaatgaca gaaaaatttt ctgatcttct gcagttcaca acacaagtct
cacgactaat ggtgacagaa attcgaagga gagcatcaaa caaatccaca gaggctgcaa
gtcgggccat agttcagttc ctagagatta atcagagtga agaagccagt agaggctgga
tgcttctaac gacaattaat ttgttagctt cctctggtca gaaaaccgtg gactgcatga
caacaatgtc agtgccttcc accctggtta aatgtttata tctgtttttt gaccttccac
atgtgcctga ggcagttgga ggtgcacaga atgagctacc tctagcagaa cgtcgaggac
tactccagaa agtttttgta cagatcttag tgaaactgtg cagttttgtt tcccctgcgg
aggagctggc tcagaaagat gatctccagc ttctattcag tgcaataacc tcttggtgcc
ctccctataa cctgccttgg agaaagagtg ctggagaagt cctcatgacc atatctcgtc
atggtcttag tgtcaatgta gtgaagtata ttcatgagaa agagtgttta tctacatgtg
ttcagaatat gcagcaatca gatgacctgt ctcccctaga aattgtcgaa atgtttgctg
ggctttcttg tttcctcaaa gattccagcg atgtttccca aacacttctg gatgattttc
ggatatggca aggatataat tttctttgtg atctcttgct tagattggaa caagcaaaag
aggcagaatc caaagatgcc ttgaaagatc tggttaatct gataacttcc ctaacaacat
atggtgtcag tgaactaaaa ccagctggta ttaccacagg ggcacccttt ttattgcctg
gatttgcagt acctcagcct gcaggcaaag gtcacagtgt gagaaacgtc caggcctttg
cagttcttca gaatgcattt ttaaaagcaa aaaccagctt ccttgcccaa atcatccttg
atgctatcac aaatatttac atggctgaca atgccaatta cttcatccta gagtcacagc
acacattgtc acagtttgca gagaagattt ctaaactccc agaagtacaa aacaaatact
ttgagatgct ggagtttgtt gtttttagct taaattatat accttgtaaa gaacttatta
gtgtcagtat cctcttaaaa tctagctctt cttatcactg tagcattatt gcaatgaaaa
cacttcttaa gtttacaaga catgactaca tatttaaaga cgtgttcagg gaggttggcc
ttttggaggt catggtaaac cttttgcata aatatgctgc cctgttgaag gatccaactc
aggcactaaa tgaacaaggg gactcaagaa ataatagttc agttgaagac caaaaacacc
tggctttatt ggttatggag accttgacag tgcttcttca aggatcaaac acaaatgcag
gaatttttcg agaatttgga ggtgcaagat gtgcacataa tatagtaaag taccctcaat
gccggcagca tgccttgatg actatccaac agctggtgct ctccccaaat ggggacgatg
acatgggcac tctcctgggg ctaatgcatt cagccccacc gacggaattg cagttgaaga
ctgatatttt aagggccctc ctgtcggtcc ttcgagaaag ccatcgttca agaacagttt
ttaggaaagt tggaggattt gtgtacatta catccttgct cgttgctatg gaaagatctt
tgagctgtcc acccaagaat ggctgggaga aagtgaacca gaatcaagtg tttgaacttc
ttcacactgt gttctgcacg ttgactgcag caatgcgcta tgagccagcc aactctcatt
tcttcaaaac agagattcag tatgagaagt tggcagatgc tgttcgattt cttggctgct
tctcagacct aagaaaaata agcgccatga atgtcttccc ctcaaataca cagccatttc
aaagactttt agaggaagat gtaatctcaa tagaatcagt gtcacccacg ttacggcact
gcagtaaact ttttatttat ctttacaaag tagccacaga ttcttttgac agtcgtgcag
aacagatccc tccttgcctg acaagtgagt cttctctccc ctctccttgg ggtacaccag
ctttgtccag gaaaaggcat gcatatcatt ctgtttcaac tccccctgtt taccctccta
aaaatgttgc cgacctgaaa ctacatgtga caacttcatc tctgcagagt tctgatgcag
tcatcattca tcctggagcc atgcttgcca tgctggacct actggcctct gttgggtcag
tgacacagcc agaacatgct ttggatcttc aacttgccgt ggcaaatatt ttacaatccc
tggtgcacac agaaaggaac cagcaagtca tgtgtgaagc tggtcttcat gcacgactgc
tgcagaggtg cagtgctgca ttggctgatg aggaccactc actgcacccg cccctgcagc
ggatgtttga acgattagcc tctcaggctc tggaacccat ggtgttgagg gagtttttac
gtttggcaag tcctttaaat tgtggtgcct gggacaaaaa actgctaaaa caatataggg
tccacaaacc aagttcactg agttatgaac cagaaatgag aagtagtatg atcacatctc
tggaaggtct gggtactgat aatgttttta gcttacatga agataaccat taccggataa
gcaagagcct ggtaaaatct gcggaaggaa gtactgtacc cctgaccagg gtgaagtgtc
tggtctccat gacaacccca catgacatca gacttcatgg gtcatcagtt actccagctt
ttgttgaatt tgacacatca cttgaagggt ttggatgtct ttttttgccc agtttggccc
ctcataatgc tcctacaaat aataccgtca caacaggtct tattgatggg gctgtggtca
gtggcattgg ttctggtgaa agattcttcc ctcctccctc cggcttaagt tactctagct
ggttttgtat tgaacatttt agttctcctc caaataacca ccctgtcaga cttcttactg
ttgtgcgccg agcaaattct tctgagcaac attacgtgtg ccttgcaata gttctatcag
caaaagaccg atctctgatt gtttccacca aagaggaact cctccaaaat tatgttgatg
attttagtga agagtcctca ttttatgaaa ttctcccatg ctgtgctcgc tttcgatgtg
gagagcttat cattgaggga cagtggcatc atttggtcct ggtaatgagc aaaggcatgt
tgaaaaacag tactgcagcc ctttatattg atggacagct tgttaacact gtaaagcttc
attatgtcca cagtactcca gggggttcag gttcggcaaa tccaccagtg gtgagcacgg
tctatgccta cattggtact ccacctgccc aacgccaaat tgcctcattg gtttggcgcc
tgggacccac acattttcta gaagaagttt taccttcttc aaatgttact accatttatg
aacttggacc aaattatgtt ggaagctttc aggctgtatg tatgccatgt aaagatgcaa
aatccgaagg ggtggtgcca tcccctgtgt cattagtacc agaggagaaa gtgtcatttg
gcctctatgc actctctgtg tcgtctctaa cagtggcaag aatccggaaa gtgtataaca
aattggatag caaagccatt gctaagcagt taggcatttc ctcacatgag aatgccactc
ctgtgaagtt gatacacaat tcagcaggac atcttaatgg atctgcacgg acaattgggg
ccgctctgat tggatacttg ggagtaagaa catttgtccc taagcctgtt gccactactt
tgcagtacgt tggtggagct gcagccatcc tgggcctggt ggccatggcc tctgatgtgg
aagggttata tgcagcagtc aaggccctgg tttgtgtggt caagagtaac ccactagcca
gcaaagaaat ggaaagaatc aagggctacc agttgctggc aatgttgctt aagaagaaac
gttcccttct taacagccac atcctccatc taactttttc tttggtggga actgttgata
gtggacatga gacctccatt attccaaatt caactgcttt ccaggacctc ctctgtgatt
ttgaagtctg gctccatgca ccatatgaac ttcatctttc cttatttgaa cactttattg
aactgctcac agagtccagt gaagcctcaa agaatgccaa attaatgaga gaattccagt
taatcccaaa gctgctcctg actcttcgag atatgtcttt atcccagcct actattgctg
ctattagtaa tgtcctgagc ttcttactgc aaggttttcc tagcagcaat gatctgctca
gatttgggca gtttatttct tctactttgc caacctttgc ggtttgtgag aaatttgtag
taatggaaat aaataatgaa gagaagcttg acactggaac tgaagaggag tttggaggtc
ttgtatcagc taatcttata cttttgagga acagacttct ggatatcttg ctaaaactaa
tttatacatc taaagaaaag acaagcatta atttgcaagc ttgtgaagaa ctggtgaaga
cactgggttt tgactggatc atgatgttta tggaggaaca cttacattcc accacagtta
cagcagccat gaggattctt gttgtcctac taagtaatca gtctattctc atcaagttta
aagaaggact cagtggtgga ggatggcttg aacagacaga ttctgtctta actaataaga
ttggaactgt attaggattc aacgtgggca gaagtgctgg tgggagatcg acggtcaggg
agattaaccg agatgcttgt cattttcctg gttttccagt ccttcagtca ttccttccta
aacacactaa tgtccctgcc ctctattttc tcctcatggc cttgtttctg cagcagccag
ttagtgagct gcctgagaac ctgcaggtca gtgtgcctgt catcagctgc cggagtaagc
agggttgcca gtttgatttg gattccattt ggacattcat ctttggagtt cctgcctcca
gcggaactgt ggtctcttct atccataacg tatgcacaga agctgttttt ttattattgg
gaatgctccg cagcatgctg acttcacctt ggcaatcaga agaagaggga tcttggctcc
gagaatatcc tgtgaccctg atgcagttct tcagatattt gtatcacaac gtgccagacc
ttgcctccat gtggatgagc cctgacttcc tgtgtgcatt agcagccacc gtcttcccct
tcaatattcg cccttactca gagatggtga ctgaccttga tgatgaagtt ggatctccag
cagaagagtt taaagcgttt gcagcagaca cagggatgaa caggagccaa tcagagtact
gcaatgtggg caccaagaca tatctgacca atcacccggc taaaaagttc gtttttgact
tcatgcgggt cttaatcata gacaacctct gtctcactcc tgccagcaag caaactccac
taattgatct tttgttggag gcttcccctg aaaggtctac aagaactcag caaaaagaat
ttcaaactta cattttggat agcgtgatgg accatttgct tgcagctgat gtgttattag
gggaagatgc atctctgcct attaccagtg gaggaagcta ccaggtattg gtgaacaatg
tgttttattt cacacagcgt gtggtggaca agctttggca aggcatgttc aacaaagaat
ctaaacttct tatagatttt ataattcaac taattgcaca gtcaaagaga agatcacagg
gattgtcact ggatgcagtg tatcattgcc tcaataggac catcttgtac cagttctcac
gggcacacaa aaccgttcct cagcaagtag ctctgcttga ttcactcagg gtcctcactg
taaacagaaa cttgatcctg ggacctggga accatgacca agaattcatt agctgtctgg
cccactgctt gataaatcta catgttggaa gcaacgtgga tggatttgga ctggaagcag
aagcccgcat gaccacatgg cacattatga tcccctcgga cattgaacca gatggtagtt
acagccaaga tattagtgaa gggcgtcagc ttctcataaa agctgtcaac agagtttgga
ctgaactgat acatagtaag aaacaagtct tagaggaact tttcaaagta actctacctg
tgaatgaaag gggccacgtg gacatagcta cagcaaggcc actcattgaa gaagctgccc
tgaagtgctg gcagaatcat ttggcccatg aaaagaaatg cataagtcga ggagaagctt
tagcgcccac cacacagtcc aaattatccc gtgtcagcag tggctttggt ctttccaagt
taacaggatc aagaaggaat cgaaaagaaa gtggtcttaa taaacacagt ctttccaccc
aggagatttc gcagtggatg tttactcaca ttgctgttgt tcgtgactta gtagatacac
aatataaaga atatcaggag cgtcagcaga atgccctgaa gtacgtgaca gaagagtggt
gtcagatcga gtgcgagctg ttgagggagc gggggctgtg gggccctccc atcggctccc
acctcgacaa gtggatgctg gagatgacag aagggccctg caggatgagg aaaaagatgg
tgcgaaatga tatgttttat aaccattacc cttacgtgcc agaaactgag caagagacaa
atgtggcgtc tgagatccca agtaaacagc ctgagacacc cgatgatatt cctcaaaaga
aacctgctcg atatagaaga gccgtaagtt atgacagtaa agagtactac atgcgactgg
cctctggcaa tcccgccatt gtccaagacg ccattgtgga gagttcagaa ggtgaagctg
ctcagcaaga accagagcat ggggaagaca ctattgctaa agtcaaaggt ttggtcaagc
ctcctctaaa acgctcccga tctgcacctg atggaggaga tgaggagaac caggagcagc
tacaagacca gattgctgag ggcagctcca tagaagagga ggagaaaaca gataatgcta
ccttactgcg cctgttagag gaaggagaaa agatccaaca catgtaccgc tgtgctcgag
tccagggcct agataccagt gaggggctcc ttctttttgg taaagagcat ttttatgtga
ttgatggatt taccatgaca gcaaccaggg aaataagaga tattgaaacc ttacctccaa
atatgcatga gcctattatt cctagaggag ccaggcaagg ccctagtcaa ctcaagagaa
catgcagcat ttttgcatat gaagatatca aggaagttca taaaaggaga tatctcctgc
agcctattgc tgtggaagtt ttctctggag atggacggaa ttacctcctt gcttttcaga
aaggaatcag aaacaaagtc tatcaaaggt ttttggctgt agtgccatct ctaacggaca
gttcagaatc tgtatctggg caacgaccaa acacgagtgt ggagcaggga tctgggttac
ttagcacttt ggttggagag aagtctgtga ctcagagatg ggagagaggt gaaatcagca
acttccaata tttgatgcat ttgaacactt tggctggcag atcatataat gatctcatgc
agtatcctgt cttcccctgg atccttgcag attatgactc agaggaggtg gatcttacta
atcccaagac gtttagaaac ctggctaagc caatgggagc acaaacagat gaacgattag
ctcagtataa gaagcggtat aaagactggg aggatcctaa tggagaaact cctgcatacc
actatgggac ccactattca tctgcaatga ttgtggcctc ataccttgta aggatggagc
ctttcacaca gatattctta aggctacagg gtggccactt tgacctggct gaccggatgt
ttcacagtgt gcgcgaggcc tggtattcag cgtcaaagca caatatggca gatgtaaaag
aacttatccc agagttcttt tatttaccag aattcctgtt caattccaac aactttgatc
taggctgtaa acaaaatggc accaagcttg gagatgttat ccttccaccc tgggcaaaag
gggacccacg agaattcatc agagtccatc gtgaggcttt ggagtgtgat tacgtgagtg
cccatctaca tgagtggatt gacttaatct tcggttataa acagcaaggc cctgctgcag
tagaagctgt aaatgtcttc catcatcttt tttatgaggg tcaagtggat atctacaaca
tcaatgaccc actaaaggag acagccacaa ttgggttcat taataacttc ggtcagatcc
ctaaacagtt atttaaaaaa cctcatccac caaagcgagt gagaagtcga ctcaatggag
acaatgcagg aatctctgtc ctaccaggat ctacaagtga caagatcttt tttcatcatc
tagacaactt gaggccttct ctaacacctg taaaagaact caaagaacct gtaggacaaa
tcgtatgtac agataaaggt attcttgcgg tggaacagaa taaggttctt atcccaccaa
cctggaataa aacttttgct tggggctatg cagacctcag ttgcagactg ggaacctatg
agtcagacaa ggccatgact gtttatgaat gcttgtctga gtggggccag attctctgtg
caatctgccc caaccccaag ctggtcatca cgggtggaac aagcacggtt gtgtgtgtgt
gggagatggg cacctccaaa gaaaaggcca agaccgtcac cctcaaacag gccttactgg
gccacactga taccgtcacc tgcgccacag catcattagc ctatcacata attgtcagtg
ggtcccgtga tcgaacctgt atcatttggg atttgaacaa actgtcattt ctaacccagc
ttcgagggca tcgagctcca gtttctgctc tttgtatcaa tgaattaaca ggggacattg
tgtcctgcgc tggcacatat atccatgtgt ggagcatcaa tgggaaccct atcgtgagtg
tcaacacgtt cacaggtagg agccagcaga tcatctgctg ctgcatgtcg gagatgaacg
aatgggacac gcagaacgtc atagtgacag gacactcaga tggagtggtt cggttttgga
gaatggaatt tttgcaagtt cctgaaacac cagctcctga gcctgctgaa gtcctagaaa
tgcaggaaga ctgtccagaa gcacaaatag ggcaggaagc ccaagacgag gacagcagtg
attcagaagc agatgagcag agcatcagcc aggaccctaa ggacactcca agccaaccca
gcagcaccag ccacaggccc cgggcagcct cctgccgcgc aacagccgcc tggtgtactg
acagtggctc tgacgactcc agacgctggt ccgaccagct cagtctagat gagaaagacg
gcttcatatt tgtgaactat tcagagggcc agaccagagc ccatctgcag ggccccctta
gccaccccca ccccaatccc attgaggtgc ggaattacag cagattgaaa cctgggtacc
gatgggaacg gcagctggtg ttcaggagta agctgactat gcacacagcc tttgatcgaa
aggacaatgc acacccagct gaggtcactg ccttgggcat ctccaaggat cacagtagga
tcctcgttgg tgacagtcga ggccgagttt tcagctggtc tgtgagtgac cagccaggcc
gttctgctgc tgatcactgg gtgaaggatg aaggtggtga cagctgctca ggctgctcgg
tgaggttttc actcacagaa agacgacacc attgcaggaa ctgtggtcag ctcttctgcc
agaagtgcag tcgctttcaa tctgaaatca aacgcttgaa aatctcatcc ccggtgcgtg
tttgtcagaa ctgttattat aacttacagc atgagagagg ttcagaagat gggcctcgaa
attgttgaag attcaacaag ctgagtggag accatggtct gtagacccct tcccgattct
cctgtcccag cttggaaggc attgaaaaca gtctccgttt acacatctct tcataccacg
tgtttgaagt gttaaaattc aaagggatca ttgaataaaa cgggtgtaga gtacaggaat
ggggcagacg cgattcaggt gaacagcaca agaagaatat gaggtggttc ctaggagcaa
cactttcgac ctccagttct ccctgatgac agtagctgtc tccaagagaa aaatcctcac
ttattaactc tcttttcttg catctcattt ttatagagct actcatcctt atttggaaaa
accaacaaca aaaaaggctt ttagaaaatg gttgtaaatc tgacttcttt gcaagtaact
atgtatattg taaatagata taaaaggcct tttttctaaa taaggactta actgcctgta
acatgaaact tcaaactaaa ccactaactc aatgaactac ttatggtttg tctgacatcc
ctcacttacc aattaattat aaatatgttt ttttaaatcc ccaaagacat tatctgtggt
ctttttttcc tttcaagctc agcctgtgtg cctgatgtca tttctttcaa gttgcccaca
gtatctccac ttaaactagg ctagtaacca aaataatgtg gaccttcttt aggaaacagt
gtgggagaat aggagtccag ccgtaagata aactggaaat atttgggcgt cttgtacctg
gctacgcacc acctcagtgt tgttcctaca taaacagggc cccttttaaa cttgtatgtg
gactgctgtt tggtcaaaga ataccttctt agcattgcag aaaggtggtc agatgaccag
tgtagtgcag gaaacagccc tgtctcaact aatggaaata tatttgcatg taacccaaaa
ttagcttatc ttgcatagaa cataataagt atgtgtcttt ggtgacacta atgttctact
atagcttatt ttcaaacaag gggtaaaaaa aggaaagaaa gaagtgtaca gaattaacat
ataaactttg ttgtaaaact gaatcatgtc agaactgctt aaaattaacc tttaccattt
aatgtcatct acctgaaaac agtgagattt atactgtatc aatgtctatt tttttgtttt
tgctatgaat ataattacag tattttaata tttagttatt taatttgttc tactagttgg
atacagaaca cacaaatcca gggggattaa agctggaagg ggctaagaga ttagtttaca
gagaaaaggc ttggtggtgg gattttttta aatgtgtgtt atgtacatat atatatatat
ataatatata ttaaaaatga aacaattaat ctagatttta acattttcag aaacttagtg
ataacattat gaacaattct aaaagccctg tgatttgaaa aatatagaat cattaatggc
ccaagatagg ccttcacacc ttcacaggtg cgaaaggaaa ggccttcaca ccctcacaga
ggcatcatgc aaaggacagc ggctttggct tttccaattt tccatcttta ggccctggtg
agaggcacac ttatgcacta aaatgcacat atatgcacat gcattcaaaa ataggcattt
ggtacaatgg tgatcttgta cctgatgggc tgaaaccagc ttaagaacaa atttgttctt
cctgatatga taactaggtc tccaagagaa aatagaaagg ctgctttagt gccttacgct
tactaaattt aaatctttat ttacctgggt ttgagcctac agtctattta tgattacata
tcaaaattga ttaaaacact tccatttcta aaagttcaaa tatacttgtt aataaaagga
ttatcggcat taatacttta atttaaagaa aagttgtgtt ctgttttcct ttctgtgtct
tactcccccc acactctccc tcccccatca ccatcttcaa ttctaataaa taatgctgat
gttcaacagt tgcagaaatt gtgctattat gtaactgtgg gccttgcccc tgtctggccc
tctagatgat ttgtagcagt gttattctac actttttaaa agaagcgtcc tccttttgtc
catgaatcat gtttacccca tacccagtgg cagaggtgtt ctttaaagac ttgaatatat
gaatgtgtgt gtgtagttac ttaaaggtta ttcctctttg taataggaaa ctatatggga
tgaacacttt taaactttcc gacacaactt ccattactaa ctttctaaca gaacttccat
aactagaagg tggaaaccaa aaccctcatg gtagtatttc ctctggcagc tggtgctgtg
ggcaactgtt ttgttcaatc gggtttcttt tctttttgcc tctaatgcag aaatcaacag
aatcactcac acatacaagt acactcacat acataaacta attatttctc tggatatctt
tctgtgttcc atgtaaattt atttaccaac atctattgtc aacatgtaca tctaccttag
tatggtctgc attctttttc tgagagtacc tcatagggct cctgcctgat ctttgtagtt
tgttcattca tccatccacc tgttcatttg ttcatccatg tattctaaca tttctatgta
gtgtgcaact ctaatgtcat gcttttgaag aagagaatag ctgcccatag cagccatccg
tctggataat agcaaaacac tctagataag ttattttgca ctttcttatg tataaagttg
gtagaaactt atttttgctt tgtatcattt aaatacattt tgttttggta aatgaactgt
gtataaaata tttatgccgt taaaactgtt tttagaaagt atttttaatt tcagcaagtt
tggttacttg ttgcatgact cttaacacag ctgacttttt gtgtcagtgc aatgtatatt
ttttgtcctg ttattaactt gtaagcccta gtaatggcca attatttgta cagcaacaga
agtaaattga agatactggc taagactgga ttgattgtgg acttttatac tatattgcag
aaaccaatat ctgtttcttg gtggttatgt aaaagacctg aagaattact atctagtgtg
cagtctgtga tatctgaatg ttcattgtat atttgtctct gatgcaaaaa ggtagagtaa,

or a nucleotide sequence having at least 80%, 85%, 90%, 92%, 95%, 96%, 97%, 98%, 99% or more sequence identity thereto which in one embodiment encodes a polypeptide having SEQ ID NO: 1 or 2, or a polypeptide with at least 80%, 85%, 90%, 95% or more, e.g., 99% or more, amino acid sequence identity thereto, or a portion thereof with the activity of SEQ ID NO: 1 or SEQ ID NO: 2.

### Delivery Vectors

Delivery vectors include, for example, nucleic acid, viral vectors, liposomes and other lipid-containing complexes, such as lipoplexes (DNA and cationic lipids), polyplexes, e.g., DNA complexed with cationic polymers such as polyethylene glycol, nanoparticles, e.g., magnetic inorganic nanoparticles that bind or are functionalized to bind DNA such as Fe₃O₄ or MnO₂ nanoparticles, microparticles, e.g., formed of polylactide polygalactide reagents, nanotubes, e.g., silica nanotubes, and other macromolecular complexes capable of mediating delivery of a gene or polypeptide, or a both, to a host cell. Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector by the cell; components that influence localization of the transferred gene within the cell after uptake (such as agents mediating nuclear localization); and components that influence expression of the gene. Such components also might include markers, such as detectable and/or selectable markers that can be used to detect or select for cells that have taken up and are expressing the nucleic acid delivered by the vector. A large variety of such vectors are known in the art and are generally available.

Gene delivery vectors include, but are not limited to, isolated nucleic acid, e.g., plasmid-based vectors which may be extrachromosomally maintained, isolated RNA, e.g., sgRNA, and viral vectors, e.g., recombinant adenovirus, retrovirus, lentivirus, herpesvirus, poxvirus, papilloma virus, or adeno-associated virus, including viral and non-viral vectors which are present in liposomes, e.g., neutral or cationic liposomes, such as DOSPA/DOPE, DOGS/DOPE or DMRIE/DOPE liposomes, and/or associated with other molecules such as DNA-anti-DNA antibody-cationic lipid (DOTMA/DOPE) complexes. Exemplary viral gene delivery vectors are described below. Gene delivery vectors may be administered via any route including, but not limited to, intracranial, intrathecal, intramuscular, buccal, rectal, intravenous or intracoronary administration, and transfer to cells may be enhanced using electroporation and/or iontophoresis, and/or scaffolding such as extracellular matrix or hydrogels, e.g., a hydrogel patch. In one embodiment, a permeation enhancer is not employed to enhance indirect delivery to the CNS.

### Retroviral Vectors

Retroviral vectors exhibit several distinctive features including their ability to stably and precisely integrate into the host genome providing long-term transgene expression. These vectors can be manipulated ex vivo to eliminate infectious gene particles to minimize the risk of systemic infection and patient-to-patient transmission. Pseudotyped retroviral vectors can alter host cell tropism.

### Lentiviruses

Lentiviruses are derived from a family of retroviruses that include human immunodeficiency virus and feline immunodeficiency virus. However, unlike retroviruses that only infect dividing cells, lentiviruses can infect both dividing and nondividing cells. Although lentiviruses have specific tropisms, pseudotyping the viral envelope with vesicular stomatitis virus yields virus with a broader range (Schnepp et al., Meth. Mol. Med., 6:427 (2002)).

### Adenoviral Vectors

Adenoviral vectors may be rendered replication-incompetent by deleting the early (E1A and E1B) genes responsible for viral gene expression from the genome and are stably maintained into the host cells in an extrachromosomal form. These vectors have the ability to transfect both replicating and nonreplicating cells and, in particular, these vectors have been shown to efficiently infect cardiac myocytes in vivo, e.g., after direction injection or perfusion. Adenoviral vectors have been shown to result in transient expression of therapeutic genes in vivo, peaking at 7 days and lasting approximately 4 weeks. The duration of transgene expression may be improved in systems utilizing neural specific promoters. In addition, adenoviral vectors can be produced at very high titers, allowing efficient gene transfer with small volumes of virus.

### Adeno-Associated Virus Vectors

Recombinant adeno-associated viruses (rAAV) are derived from nonpathogenic parvoviruses, evoke essentially no cellular immune response, and produce transgene expression lasting months in most systems. Moreover, like adenovirus, adeno-associated virus vectors also have the capability to infect replicating and nonreplicating cells and are believed to be nonpathogenic to humans.

AAV vectors include but are not limited to AAV1, AAV2, AAV5, AAV7, AAV8, AAV9 or AAVrh.10.

### Plasmid DNA Vectors

Plasmid DNA is often referred to as “naked DNA” to indicate the absence of a more elaborate packaging system. Direct injection of plasmid DNA to myocardial cells in vivo has been accomplished. Plasmid-based vectors are relatively nonimmunogenic and nonpathogenic, with the potential to stably integrate in the cellular genome, resulting in long-term gene expression in postmitotic cells in vivo. Plasmid DNA may be delivered to cells as part of a macromolecular complex, e.g., a liposome or DNA-protein complex, and delivery may be enhanced using techniques including electroporation.

Where translation is also desired in the intended target cell, the heterologous polynucleotide may also comprise control elements that facilitate translation (such as a ribosome binding site or “RBS” and a polyadenylation signal). Accordingly, the heterologous polynucleotide generally comprises at least one coding region operatively linked to a suitable promoter, and may also comprise, for example, an operatively linked enhancer, ribosome binding site and poly-A signal. The heterologous polynucleotide may comprise one encoding region, or more than one encoding regions under the control of the same or different promoters. The entire unit, containing a combination of control elements and encoding region, is often referred to as an expression cassette.

### Exemplary Non-Viral Delivery Vehicles

Biodegradable particles comprising, e.g., isolated nucleic acid or a vector or a polypeptide, or a combination thereof, may include or may be formed from biodegradable polymeric molecules which may include, but are not limited to polylactic acid (PLA), polyglycolic acid (PGA), co-polymers of PLA and PGA (i.e., polyactic-co-glycolic acid (PLGA)), poly-ε-caprolactone (PCL), polyethylene glycol (PEG), poly(3-hydroxybutyrate), poly(p-dioxanone), polypropylene fumarate, poly(orthoesters), polyol/diketene acetals addition polymers, poly-alkyl-cyano-acrylates (PAC), poly(sebacic anhydride) (PSA), poly(carboxybiscarboxyphenoxyphenoxy hexone (PCPP) poly[bis (p-carboxypheonoxy)methane](PCPM), copolymers of PSA, PCPP and PCPM, poly(amino acids), poly(pseudo amino acids), polyphosphazenes, derivatives of poly[(dichloro)phosphazenes] and poly[(organo)phosphazenes], poly-hydroxybutyric acid, or S-caproic acid, elastin, or gelatin. (See, e.g., Kumari et al., Colloids and Surfaces B: Biointerfaces 75 (2010) 1-18; and U.S. Pat. Nos. 6,913,767; 6,884,435; 6,565,777; 6,534,092; 6,528,087; 6,379,704; 6,309,569; 6,264,987; 6,210,707; 6,090,925; 6,022,564; 5,981,719; 5,871,747; 5,723,269; 5,603,960; and 5,578,709; and U.S. Published Application No. 2007/0081972; and International Application Publication Nos. WO 2012/115806; and WO 2012/054425; the contents of which are incorporated herein by reference in their entireties).

The biodegradable nanoparticles may be prepared by methods known in the art. (See, e.g., Nagavarma et al., Asian J. of Pharma. And Clin. Res., Vol 5, Suppl 3, 2012. pages 16-23; Cismaru et al., Rev. Roum. Chim., 2010, 55(8), 433-442; and International Application Publication Nos. WO 2012/115806; and WO 2012/054425; the contents of which are incorporated herein by reference in their entireties). Suitable methods for preparing the nanoparticles may include methods that utilize a dispersion of a preformed polymer. which may include but are not limited to solvent evaporation, nanoprecipitation, emulsification/solvent diffusion, salting out, dialysis, and supercritical fluid technology. In some embodiments, the nanoparticles may be prepared by forming a double emulsion (e.g., water-in-oil-in-water) and subsequently performing solvent-evaporation. The nanoparticles obtained by the disclosed methods may be subjected to further processing steps such as washing and lyophilization, as desired. Optionally, the nanoparticles may be combined with a preservative (e.g., trehalose).

Typically, the nanoparticles have a mean effective diameter of less than 1 micron. e.g., the nanoparticles have a mean effective diameter of between about 25 nm and about 500 nm, e.g., between about 50 nm and about 250 nm. about 100 nm to about 150 nm, or about 450 nm to 650 nm. The size of the particles (e.g., mean effective diameter) may be assessed by known methods in the art, which may include but are not limited to transmission electron microscopy (TEM), scanning electron microscopy (SEM), Atomic Force Microscopy (AFM), Photon Correlation Spectroscopy (PCS), Nanoparticle Surface Area Monitor (NSAM), Condensation Particle Counter (CPC), Differential Mobility Analyzer (DMA), Scanning Mobility Particle Sizer (SMPS), Nanoparticle Tracking Analysis (NTA), X-Ray Diffraction (XRD), Aerosol Time of Flight Mass Spectroscopy (ATFMS), and Aerosol Particle Mass Analyzer (APM).

The biodegradable nanoparticles may have a zeta-potential that facilitates uptake by a target cell. Typically, the nanoparticles have a zeta-potential greater than 0. In some embodiments, the nanoparticles have a zeta-potential between about 5 mV to about 45 mV, between about 15 mV to about 35 mV, or between about 20 mV and about 40 mV. Zeta-potential may be determined via characteristics that include electrophoretic mobility or dynamic electrophoretic mobility. Electrokinetic phenomena and electroacoustic phenomena may be utilized to calculate zeta-potential.

In one embodiment, a non-viral delivery vehicle comprises polymers including but not limited to poly(lactic-co-glycolic acid) (PLGA), polylactic acid (PLA), linear and/or branched PEI with differing molecular weights (e.g., 2, 22 and 25 kDa), dendrimers such as polyamidoamine (PAMAM) and polymethoacrylates; lipids including but not limited to cationic liposomes, cationic emulsions, DOTAP, DOTMA, DMRIE, DOSPA, distearoylphosphatidylcholine (DSPC), DOPE, or DC-cholesterol; peptide based vectors including but not limited to Poly-L-lysine or protamine; or poly(β-amino ester), chitosan, PEI-polyethylene glycol, PEI-mannose-dextrose, DOTAP-cholesterol or RNAiMAX.

In one embodiment, the delivery vehicle is a glycopolymer-based delivery vehicle, poly(glycoamidoamine)s (PGAAs), that have the ability to complex with various polynucleotide types and form nanoparticles. These materials are created by polymerizing the methylester or lactone derivatives of various carbohydrates (D-glucarate (D), meso-galactarate (G), D-mannarate (M), and L-tartarate (T)) with a series of oligoethyleneamine monomers (containing between 1-4 ethylenamines (Liu and Reineke, 2006). A subset composed of these carbohydrates and four ethyleneamines in the polymer repeat units yielded exceptional delivery efficiency.

In one embodiment, the delivery vehicle comprises polyethyleneimine (PEI), Polyamidoamine (PAMAM), PEI-PEG, PEI-PEG-mannose, dextran-PEI, OVA conjugate, PLGA microparticles, or PLGA microparticles coated with PAMAM, or any combination thereof. The disclosed cationic polymer may include, but are not limited to, polyamidoamine (PAMAM) dendrimers. Polyamidoamine dendrimers suitable for preparing the presently disclosed nanoparticles may include 3rd-, 4th-, 5th-, or at least 6th-generation dendrimers.

In one embodiment, the delivery vehicle comprises a lipid, e.g., N-[1-(2,3-dioleoyloxy)propel]-N,N,N-trimethylammonium (DOTMA), 2,3-dioleyloxy-N-[2-spermine carboxamide]ethyl-N,N-dimethyl-1-propanammonium trifluoracetate (DOSPA, Lipofectamine); 1,2-dioleoyl-3-trimethylammonium-propane (DOTAP); N-[1-(2,3-dimyristloxy) propyl]; N,N-dimethyl-N-(2-hydroxyethyl) ammonium bromide (DMRIE), 3-β-[N—(N,N-dimethylaminoethane) carbamoyl]cholesterol (DC-Chol); dioctadecyl amidoglyceryl spermine (DOGS, Transfectam); or imethyldioctadeclyammonium bromide (DDAB). The positively charged hydrophilic head group of cationic lipids usually consists of monoamine such as tertiary and quaternary amines, polyamine, amidinium, or guanidinium group. A series of pyridinium lipids have been developed (Zhu et al., 2008; van der Woude et al., 1997; Ilies et al., 2004). In addition to pyridinium cationic lipids, other types of heterocyclic head group include imidazole, piperizine and amino acid. The main function of cationic head groups is to condense negatively charged nucleic acids by means of electrostatic interaction to slightly positively charged nanoparticles, leading to enhanced cellular uptake and endosomal escape.

Lipids having two linear fatty acid chains, such as DOTMA, DOTAP and SAINT-2, or DODAC, may be employed as a delivery vehicle, as well as tetraalkyl lipid chain surfactant, the dimer of N,N-dioleyl-N,N-dimethylammonium chloride (DODAC). All the trans-orientated lipids regardless of their hydrophobic chain lengths (C_{16:1}, C_{18:1} and C_{20:1}) appear to enhance the transfection efficiency compared with their cis-orientated counterparts.

The structures of cationic polymers useful as a delivery vehicle include but are not limited to linear polymers such as chitosan and linear poly(ethyleneimine), branched polymers such as branch poly(ethyleneimine) (PEI), circle-like polymers such as cyclodextrin, network (crosslinked) type polymers such as crosslinked poly(amino acid) (PAA), and dendrimers. Dendrimers consist of a central core molecule, from which several highly branched arms ‘grow’ to form a tree-like structure with a manner of symmetry or asymmetry. Examples of dendrimers include polyamidoamine (PAMAM) and polypropylenimine (PPI) dendrimers.

DOPE and cholesterol are commonly used neutral co-lipids for preparing cationic liposomes. Branched PEI-cholesterol water-soluble lipopolymer conjugates self-assemble into cationic micelles. Pluronic (poloxamer), a non-ionic polymer and SP1017, which is the combination of Pluronics L61 and F127, may also be used.

In one embodiment, PLGA particles are employed to increase the encapsulation frequency although complex formation with PLL may also increase the encapsulation efficiency. Other cationic materials, for example, PEI, DOTMA, DC-Chol, or CTAB, may be used to make nanospheres.

In one embodiment, complexes are embedded in or applied to a material including but not limited to hydrogels of poloxamers, polyacrylamide, poly(2-hydroxyethyl methacrylate), carboxyvinyl-polymers (e.g., Carbopol 934, Goodrich Chemical Co.), cellulose derivatives, e.g., methylcellulose, cellulose acetate and hydroxypropyl cellulose, polyvinyl pyrrolidone or polyvinyl alcohols, or combinations thereof.

In some embodiments, a biocompatible polymeric material is derived from a biodegradable polymeric such as collagen, e.g., hydroxylated collagen, fibrin, polylactic-polyglycolic acid, or a polyanhydride. Other examples include, without limitation, any biocompatible polymer, whether hydrophilic, hydrophobic, or amphiphilic, such as ethylene vinyl acetate copolymer (EVA), polymethyl methacrylate, polyamides, polycarbonates, polyesters, polyethylene, polypropylenes, polystyrenes, polyvinyl chloride, polytetrafluoroethylene, N-isopropylacrylamide copolymers, poly(ethylene oxide)/poly(propylene oxide) block copolymers, poly(ethylene glycol)/poly(D,L-lactide-co-glycolide) block copolymers, polyglycolide, polylactides (PLLA or PDLA), poly(caprolactone) (PCL), or poly(dioxanone) (PPS).

In another embodiment, the biocompatible material includes polyethyleneterephalate, polytetrafluoroethylene, copolymer of polyethylene oxide and polypropylene oxide, a combination of polyglycolic acid and polyhydroxyalkanoate, gelatin, alginate, poly-3-hydroxybutyrate, poly-4-hydroxybutyrate, and polyhydroxyoctanoate, and polyacrylonitrilepolyvinylchlorides.

In one embodiment, the following polymers may be employed, e.g., natural polymers such as starch, chitin, glycosaminoglycans, e.g., hyaluronic acid, dermatan sulfate and chrondrotin sulfate, and microbial polyesters, e.g., hydroxyalkanoates such as hydroxyvalerate and hydroxybutyrate copolymers, and synthetic polymers, e.g., poly(orthoesters) and polyanhydrides, and including homo and copolymers of glycolide and lactides (e.g., poly(L-lactide, poly(L-lactide-co-D,L-lactide), poly(L-lactide-co-glycolide, polyglycolide and poly(D,L-lactide), pol(D,L-lactide-coglycolide), poly(lactic acid colysine) and polycaprolactone.

In one embodiment, the biocompatible material is derived from isolated extracellular matrix (ECM). ECM may be isolated from endothelial layers of various cell populations, tissues and/or organs, e.g., any organ or tissue source including the dermis of the skin, liver, alimentary, respiratory, intestinal, urinary or genital tracks of a warm-blooded vertebrate. ECM employed in the disclosure may be from a combination of sources. Isolated ECM may be prepared as a sheet, in particulate form, gel form and the like.

The biocompatible scaffold polymer may comprise silk, elastin, chitin, chitosan, poly(d-hydroxy acid), poly(anhydrides), or poly(orthoesters). More particularly, the biocompatible polymer may be formed polyethylene glycol, poly(lactic acid), poly(glycolic acid), copolymers of lactic and glycolic acid, copolymers of lactic and glycolic acid with polyethylene glycol, poly(E-caprolactone), poly(3-hydroxybutyrate), poly(p-dioxanone), polypropylene fumarate, poly(orthoesters), polyol/diketene acetals addition polymers, poly(sebacic anhydride) (PSA), poly(carboxybiscarboxyphenoxyphenoxy hexone (PCPP) poly[bis (p-carboxypheonoxy) methane](PCPM), copolymers of SA, CPP and CPM, poly(amino acids), poly(pseudo amino acids), polyphosphazenes, derivatives of poly[(dichloro)phosphazenes] or poly[(organo) phosphazenes], poly-hydroxybutyric acid, or S-caproic acid, polylactide-co-glycolide, polylactic acid, polyethylene glycol, cellulose, oxidized cellulose, alginate, gelatin or derivatives thereof.

Thus, the polymer may be formed of any of a wide range of materials including polymers, including naturally occurring polymers, synthetic polymers, or a combination thereof. In one embodiment, the scaffold comprises biodegradable polymers. In one embodiment, a naturally occurring biodegradable polymer may be modified to provide for a synthetic biodegradable polymer derived from the naturally occurring polymer. In one embodiment, the polymer is a poly(lactic acid) (“PLA”) or poly(lactic-co-glycolic acid) (“PLGA”). In one embodiment, the scaffold polymer includes but is not limited to alginate, chitosan, poly(2-hydroxyethylmethacrylate), xyloglucan, co-polymers of 2-methacryloyloxyethyl phosphorylcholine, poly(vinyl alcohol), silicone, hydrophobic polyesters and hydrophilic polyester, poly(lactide-co-glycolide), N-isoproylacrylamide copolymers, poly(ethylene oxide)/poly(propylene oxide), polylactic acid, poly(orthoesters), polyanhydrides, polyurethanes, copolymers of 2-hydroxyethylmethacrylate and sodium methacrylate, phosphorylcholine, cyclodextrins, polysulfone and polyvinylpyrrolidine, starch, poly-D,L-lactic acid-para-dioxanone-polyethylene glycol block copolymer, polypropylene, poly(ethylene terephthalate), poly(tetrafluoroethylene), poly-epsilon-caprolactone, or crosslinked chitosan hydrogels.

### Exemplary Lipids

Numerous lipids which are used in liposome delivery systems may be used. Virtually any lipid or polymer which is used to form a liposome or polymersome may be used. Exemplary lipids for use include, for example, 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine](DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000](18:1 PEG-2000 PE), 1,2-dipalmitoyl-sn-glycero-3-phosphoethanolamine-N-[methoxy(polyethylene glycol)-2000](16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof. Cholesterol, not technically a lipid, but presented as a lipid for purposes of an embodiment of the given the fact that cholesterol may be an important component of the lipid according to an embodiment. Often cholesterol is incorporated into lipid particles in order to enhance structural integrity. These lipids are all readily available commercially from Avanti Polar Lipids, Inc. (Alabaster, Alabama, USA). DOPE and DPPE are particularly useful for conjugating (through an appropriate crosslinker) peptides, polypeptides, including antibodies, RNA and DNA through the amine group on the lipid.

In certain embodiments, the lipid is comprised of one or more lipids selected from the group consisting of phosphatidyl-cholines (PCs) and cholesterol.

In certain embodiments, the lipid is comprised of one or more phosphatidyl-cholines (PCs) selected from the group consisting of 1,2-distearoyl-sn-glycero-3-phosphocholine (DSPC) [18:0], 1,2-dioleoyl-sn-glycero-3-phosphocholine (DOPC) [18:1 (Δ9-Cis)], 1,2-dimyristoyl-sn-glycero-3-phosphocholine (DMPC), 1,2-dioleoyl-3-trimethylammonium-propane (DOTAP), 1-palmitoyl-2-oleoyl-sn-glycero-3-phosphocholine (POPC), egg PC, and a lipid mixture comprising of one or more unsaturated phosphatidyl-cholines, DMPC [14:0] having a carbon length of 14 and no unsaturated bonds, 1,2-dipalmitoyl-sn-glycero-3-phosphocholine (DPPC) [16:0], POPC [16:0-18:1], and DOTAP [18:1]. The use of DSPC and/or DOPC as well as other zwitterionic phospholipids as a principal component (often in combination with a minor amount of cholesterol) is employed in certain embodiments in order to provide a protocell with a surface zeta potential which is neutral or close to neutral in character.

In other embodiments: (a) the lipid is comprised of a mixture of (1) DSPC, DOPC and optionally one or more phosphatidyl-cholines (PCs) selected from the group consisting of 1,2-dimyristoyl-sn-glycero-3-phosphocholine (DMPC), 1,2-dioleoyl-3-trimethylammonium-propane (DOTAP), 1-palmitoyl-2-oleoyl-sn-glycero-3-phosphocholine (POPC), a lipid mixture comprising (in molar percent) between about 50% to about 70% or about 51% to about 69%, or about 52% to about 68%, or about 53% to about 67%, or about 54% to about 66%, or about 55% to about 65%, or about 56% to about 64%, or about 57% to about 63%, or about 58% to about 62%, or about 59% to about 61%, or about 60%, of one or more unsaturated phosphatidyl-choline, DMPC [14:0] having a carbon length of 14 and no unsaturated bonds, 1,2-dipalmitoyl-sn-glycero-3-phosphocholine (DPPC) [16:0], POPC [16:0-18:1] and DOTAP [18:1]; and wherein (b) the molar concentration of DSPC and DOPC in the mixture is between about 10% to about 99% or about 50% to about 99%, or about 12% to about 98%, or about 13% to about 97%, or about 14% to about 96%, or about 55% to about 95%, or about 56% to about 94%, or about 57% to about 93%, or about 58% to about 42%, or about 59% to about 91%, or about 50% to about 90%, or about 51% to about 89%.

In certain embodiments, the lipid is comprised of one or more compositions selected from the group consisting of a phospholipid, a phosphatidyl-choline, a phosphatidyl-serine, a phosphatidyl-diethanolamine, a phosphatidylinosite, a sphingolipid, and an ethoxylated sterol, or mixtures thereof. In illustrative examples of such embodiments, the phospholipid can be a lecithin; the phosphatidylinosite can be derived from soy, rape, cotton seed, egg and mixtures thereof; the sphingolipid can be ceramide, a cerebroside, a sphingosine, and a sphingomyelin, and a mixture thereof; the ethoxylated sterol can be phytosterol, PEG-(polyethyleneglycol)-5-soy bean sterol, and PEG-(polyethyleneglycol)-5 rapeseed sterol. In certain embodiments, the phytosterol comprises a mixture of at least two of the following compositions: sitosterol, campesterol and stigmasterol.

In still other illustrative embodiments, the lipid is comprised of one or more phosphatidyl groups selected from the group consisting of phosphatidyl choline, phosphatidyl-ethanolamine, phosphatidyl-serine, phosphatidyl-inositol, lyso-phosphatidyl-choline, lyso-phosphatidyl-ethanolamine, lyso-phosphatidyl-inositol and lyso-phosphatidyl-inositol.

In still other illustrative embodiments, the lipid nanoparticle is comprised of phospholipid selected from a monoacyl or diacylphosphoglyceride.

In still other illustrative embodiments, the lipid is comprised of one or more phosphoinositides selected from the group consisting of phosphatidyl-inositol-3-phosphate (PI-3-P), phosphatidyl-inositol-4-phosphate (PI-4-P), phosphatidyl-inositol-5-phosphate (PI-5-P), phosphatidyl-inositol-3,4-diphosphate (PI-3,4-P2), phosphatidyl-inositol-3,5-diphosphate (PI-3,5-P2), phosphatidyl-inositol-4,5-diphosphate (PI-4,5-P2), phosphatidyl-inositol-3,4,5-triphosphate (PI-3,4,5-P3), lysophosphatidyl-inositol-3-phosphate (LPI-3-P), lysophosphatidyl-inositol-4-phosphate (LPI-4-P), lysophosphatidyl-inositol-5-phosphate (LPI-5-P), lysophosphatidyl-inositol-3,4-diphosphate (LPI-3,4-P2), lysophosphatidyl-inositol-3,5-diphosphate (LPI-3,5-P2), lysophosphatidyl-inositol-4,5-diphosphate (LPI-4,5-P2), and lysophosphatidyl-inositol-3,4,5-triphosphate (LPI-3,4,5-P3), and phosphatidyl-inositol (PI), and lysophosphatidyl-inositol (LPI). In still other illustrative embodiments, the lipid is comprised of one or more phospholipids selected from the group consisting of PEG-poly(ethylene glycol)-derivatized distearoylphosphatidylethanolamine (PEG-DSPE), PEG-poly(ethylene glycol)-derivatized dioleoylphosphatidylethanolamine (PEG-DOPE), poly(ethylene glycol)-derivatized ceramides (PEG-CER), hydrogenated soy phosphatidylcholine (HSPC), egg phosphatidylcholine (EPC), phosphatidyl ethanolamine (PE), phosphatidyl glycerol (PG), phosphatidyl inositol (PI), monosialoganglioside, sphingomyelin (SPM), distearoylphosphatidylcholine (DSPC), dimyristoylphosphatidylcholine (DMPC), and dimyristoylphosphatidylglycerol (DMPG).

In still other embodiments, the lipid comprises one or more PEG-containing phospholipids, for example 1,2-dioleoyl-sn-glycero-3-phosphoethanolamine-N-[methoxy(polyethylene glycol)](ammonium salt) (DOPE-PEG), 1,2-distearoyl-sn-glycero-3-phosphoethanolamine-N-[methoxy(polyethylene glycol)](ammonium salt) (DSPE-PEG), 1,2-distearoyl-sn-glycero-3-phosphoethanolamine-N-[amino(polyethylene glycol)](DSPE-PEG-NH₂) (DSPE-PEG). In the PEG-containing phospholipid, the PEG group ranges from about 2 to about 250 ethylene glycol units, about 5 to about 100, about 10 to 75, or about 40-50 ethylene glycol units. In certain exemplary embodiments, the PEG-phospholipid is 1,2-dioleoyl-sn-glycero-3-phosphoethanolamine-N-[methoxy(polyethylene glycol)-2000](ammonium salt) (DOPE-PEG₂₀₀₀), 1,2-distearoyl-sn-glycero-3-phosphoethanolamine-N-[methoxy(polyethylene glycol)-2000](ammonium salt) (DSPE-PEG₂₀₀₀), 1,2-distearoyl-sn-glycero-3-phosphoethanolamine-N-[amino(polyethylene glycol)-2000](DSPE-PEG₂₀₀₀-NH₂) which can be used to covalent bind a functional moiety to the lipid.

In on embodiment, the lipid particle comprises one of more lipids selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine](DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000](18:1 PEG-2000 PE), 1,2-dipalmitoyl-sn-glycero-3-phosphoethanolamine-N-[methoxy(polyethylene glycol)-2000](16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof.

In one embodiment, pharmaceutical compositions described herein may include, without limitation, lipids such as 1,2-dioleyloxy-N,N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (US20100324120; herein incorporated by reference in its entirety) and liposomes which may deliver small molecule drugs such as, but not limited to, DOXIL® from Janssen Biotech, Inc. (Horsham, Pa.). In one embodiment, the cationic lipid may be selected from, but not limited to, a cationic lipid described in International Publication Nos. WO2012040184, WO2011153120, WO2011149733, WO2011090965, WO2011043913, WO2011022460, WO2012061259, WO2012054365, WO2012044638, WO2010080724, WO201021865 and WO2008103276, U.S. Pat. Nos. 7,893,302, 7,404,969 and 8,283,333 and US Patent Publication No. US20100036115 and US20120202871; each of which is herein incorporated by reference in their entirety. In another embodiment, the cationic lipid may be selected from, but not limited to, formula A described in International Publication Nos. WO2012040184, WO2011153120, WO2011149733, WO2011090965, WO2011043913, WO2011022460, WO2012061259, WO2012054365 and WO2012044638; each of which is herein incorporated by reference in their entirety. In yet another embodiment, the cationic lipid may be selected from, but not limited to, formula CLI-CLXXIX of International Publication No. WO2008103276, formula CLI-CLXXIX of U.S. Pat. No. 7,893,302, formula CLI-CLXXXXII of U.S. Pat. No. 7,404,969 and formula I-VI of US Patent Publication No. US20100036115; each of which is herein incorporated by reference in their entirety. As a non-limiting example, the cationic lipid may be selected from (20Z,23Z)—N,N-dimethylnonacosa-20,23-dien-10-amine, (17Z,20Z)—N,N-dimemylhexacosa-17,20-dien-9-amine, (1Z,19Z)—N5N-dimethylpentacosa-16,19-dien-8-amine, (13Z,16Z)—N,N-dimethyldocosa-13,16-dien-5-amine, (12Z,15Z)—N,N-dimethylhenicosa-12,15-dien-4-amine, (14Z,17Z)—N,N-dimethyltricosa-14,17-dien-6-amine, (15Z,18Z)—N,N-dimethyltetracosa-15,18-dien-7-amine, (18Z,21Z)—N,N-dimethylheptacosa-18,21-dien-10-amine, (15Z,18Z)—N,N-dimethyltetracosa-15,18-dien-5-amine, (14Z,17Z)—N,N-dimethyltricosa-14,17-dien-4-amine, (19Z,22Z)—N,N-dimeihyloctacosa-19,22-dien-9-amine, (18Z,21 Z)—N,N-dimethylheptacosa-18,21-dien-8-amine, (17Z,20Z)—N,N-dimethylhexacosa-17,20-dien-7-amine, (16Z,19Z)—N,N-dimethylpentacosa-16,19-dien-6-amine, (22Z,25Z)—N,N-dimethylhentriaconta-22,25-dien-10-amine, (21Z,24Z)—N,N-dimethyltriaconta-21,24-dien-9-amine, (18Z)—N,N-dimetylheptacos-18-en-10-amine, (17Z)—N,N-dimethylhexacos-17-en-9-amine, (19Z,22Z)—N,N-dimethyloctacosa-19,22-dien-7-amine, N,N-dimethylheptacosan-10-amine, (20Z,23Z)—N-ethyl-N-methylnonacosa-20,23-dien-10-amine, 1-[(11Z,14Z)-1-nonylicosa-11,14-dien-1-yl]pyrrolidine, (20Z)—N,N-dimethylheptacos-20-en-10-amine, (15Z)—N,N-dimethyl eptacos-15-en-10-amine, (14Z)—N,N-dimethylnonacos-14-en-10-amine, (17Z)—N,N-dimethylnonacos-17-en-10-amine, (24Z)—N,N-dimethyltritriacont-24-en-10-amine, (20Z)—N,N-dimethylnonacos-20-en-10-amine, (22Z)—N,N-dimethylhentriacont-22-en-10-amine, (16Z)—N,N-dimethylpentacos-16-en-8-amine, (12Z,15Z)—N,N-dimethyl-2-nonylhenicosa-12,15-dien-1-amine, (13Z,16Z)—N,N-dimethyl-3-nonyldocosa-13,16-dien-1-amine, N,N-dimethyl-1-[(1S,2R)-2-octylcyclopropyl]eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine,N,N-dimeth-yl-1-[(1S,2S)-2-{[(1R,2R)-2-pentylcyclopropyl]methyl}cyclopropyl]nonadecan-10-amine,N,N-dimethyl-1-[(1S,2R)-2-octylcyclopropyl]hexadecan-8-amine, N,N-dimethyl-[(1R,2S)-2-undecylcyclopropyl]tetradecan-5-amine, N,N-dimethyl-3-{7-[(1S,2R)-2-octylcyclopropyl]heptyl}dodecan-1-amine, 1-[(1R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-[(1S,2R)-2-octylcyclopropyl]pentadecan-8-amine, R—N,N-dimethyl-1-[(9Z,12Z)-octadeca-9,12-dien-1-yloxy]-3-(octyloxy)propa-n-2-amine, S—N,N-dimethyl-1-[(9Z,12Z)-octadeca-9,12-dien-1-yloxy]-3-(octy-loxy)propan-2-amine, 1-{2-[(9Z,12Z)-octadeca-9,12-dien-1-yloxy]-1-[(octyloxy)methyl]ethyl}pyrr-olidine, (2S)—N,N-dimethyl-1-[(9Z,12Z)-octadeca-9,12-dien-1-yloxy]-3-[(5Z-)-oct-5-en-1-yloxy]propan-2-amine, 1-{2-[(9Z,12Z)-octadeca-9,12-dien-1-yloxy]-1-[(octyloxy)methyl]ethyl}azet-idine, (2S)-1-(hexyloxy)-N,N-dimethyl-3-[(9Z,12Z)-octadeca-9,12-dien-1-ylo-xy]propan-2-amine, (2S)-1-(heptyloxy)-N,N-dimethyl-3-[(9Z,12Z)-octadeca-9,12-dien-1-yloxy]pr-opan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-am-ine; (2S)—N,N-dimethyl-1-[(6Z,9Z,12Z)-octadeca-6,9,12-trien-1-yloxy]-3-(o-ctyloxy)propan-2-amine, (2S)-1-[(11Z,14Z)-icosa-11,14-dien-1-yloxy]-N,N-dimethyl-3-(pentyloxy)pro-pan-2-amine, (2S)-1-(hexyloxy)-3-[(11Z,14Z)-icosa-11,14-dien-1-yloxy]-N,N-dimethylprop-an-2-amine, 1-[(11Z,14Z)-icosa-11,14-dien-1-yloxy]-N,N-dimethyl-3-(octyloxy)propan-2-amine, 1-[(13Z,16Z)-docosa-13,16-dien-1-yloxy]-N,N-dimethyl-3-(octyloxy)pr-opan-2-amine, (2S)-1-[(13Z,16Z)-docosa-13,16-dien-1-yloxy]-3-(hexyloxy)-N,N-dimethylpro-pan-2-amine, (2S)-1-[(13Z)-docos-13-en-1-yloxy]-3-(hexyloxy)-N,N-dimethylpropan-2-amin-e, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)—N,N-dimethyl-H(1-metoylo ctyl)oxy]-3-[(9Z,12Z)-octadeca-9,12-dien-1-yloxy]propan-2-amine, (2R)-1-[(3,7-dimethyloctyl)oxy]-N,N-dimethyl-3-[(9Z,12Z)-octadeca-9,12-di-en-1-yloxy]propan-2-amine, N,N-dimethyl-1-(octyloxy)-3-({8-[(1S,2S)-2-{[(1R,2R)-2-pentylcyclopropyl]-methyl}cyclopropyl]octyl}oxy)propan-2-amine, N,N-dimethyl-1-{[8-(2-oclylcyclopropyl)octyl]oxy}-3-(octyloxy)propan-2-amine and (11E,20Z,23Z)—N,N-dimethylnonacosa-11,20,2-trien-10-amine or a pharmaceutically acceptable salt or stereoisomer thereof.

In one embodiment, the LNP may contain PEG-DMG 2000 (1,2-dimyristoyl-sn-glycero-3-phophoethanolamine-N-[methoxy(polyethylene glycol)-2000). In one embodiment, the LNP formulation may contain PEG-DMG 2000, a cationic lipid known in the art and at least one other component. In another embodiment, the LNP formulation may contain PEG-DMG 2000, a cationic lipid known in the art, DSPC and cholesterol. As a non-limiting example, the LNP formulation may contain PEG-DMG 2000, DLin-DMA, DSPC and cholesterol. As another non-limiting example, the LNP formulation may contain PEG-DMG 2000, DLin-DMA, DSPC and cholesterol in a molar ratio of 2:40:10:48 (see e.g., Geall et al., Nonviral delivery of self-amplifying RNA vaccines, PNAS 2012; PMID: 22908294; herein incorporated by reference in its entirety).

In one embodiment, the LNP may include MC3.

### Pharmaceutical Compositions

The disclosure provides a composition comprising, consisting essentially of, or consisting of the above-described vector and/or isolated nucleic acid and/or isolated polypeptide, and a pharmaceutically acceptable (e.g., physiologically acceptable) carrier. When the composition consists essentially of the vector and/or isolated nucleic acid and/or isolated polypeptide, and a pharmaceutically acceptable carrier, additional components can be included that do not materially affect the composition (e.g., adjuvants, buffers, stabilizers, anti-inflammatory agents, solubilizers, preservatives, etc.). When the composition consists of the vector and/or isolated nucleic acid and/or isolated polypeptide, and the pharmaceutically acceptable carrier, the composition does not comprise any additional components. Any suitable carrier can be used within the context of the disclosure, and such carriers are well known in the art. The choice of carrier will be determined, in part, by the particular site to which the composition may be administered and the particular method used to administer the composition. The composition optionally can be sterile with the exception of the gene transfer vector described herein. The composition can be frozen or lyophilized for storage and reconstituted in a suitable sterile carrier prior to use. The compositions can be generated in accordance with conventional techniques described in, e.g., Remington: The Science and Practice of Pharmacy, 21st Edition, Lippincott Williams & Wilkins, Philadelphia, PA (2001).

Suitable formulations for the composition include aqueous and non-aqueous solutions, isotonic sterile solutions, which can contain antioxidants, buffers, and bacteriostats, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets of the kind previously described. In one embodiment, the carrier is a buffered saline solution. In one embodiment, the inventive gene transfer vector is administered in a composition formulated to protect the gene transfer vector from damage prior to administration. For example, the composition can be formulated to reduce loss of the vector and/or isolated nucleic acid and/or isolated polypeptide, on devices used to prepare, store, or administer the gene transfer vector, such as glassware, syringes, or needles. The composition can be formulated to decrease the light sensitivity and/or temperature sensitivity of the vector and/or isolated nucleic acid and/or isolated polypeptide. To this end, the composition may comprise a pharmaceutically acceptable liquid carrier, such as, for example, those described above, and a stabilizing agent selected from the group consisting of polysorbate 80, L-arginine, polyvinylpyrrolidone, trehalose, and combinations thereof. Use of such a composition will extend the shelf life of the vector, and/or isolated nucleic acid and/or isolated polypeptide, facilitate administration, and increase the efficiency of the i method. Formulations for gene transfer vector-containing compositions are further described in, for example, Wright et al., Curr. Opin. Drug Discov. Devel., 6(2):174-178 (2003) and Wright et al., Molecular Therapy, 12: 171-178 (2005))

The composition also can be formulated to enhance transduction efficiency. In addition, one of ordinary skill in the art will appreciate that the vector and/or isolated nucleic acid and/or isolated polypeptide, can be present in a composition with other therapeutic or biologically active agents. For example, factors that control inflammation, such as ibuprofen or steroids, can be part of the composition to reduce swelling and inflammation associated with in vivo administration of the gene transfer vector. Immune system stimulators or adjuvants, e.g., interleukins, lipopolysaccharide, and double-stranded RNA. Antibiotics, i.e., microbicides and fungicides, can be present to treat existing infection and/or reduce the risk of future infection, such as infection associated with gene transfer procedures.

Injectable depot forms are made by forming microencapsule matrices of the subject compounds in biodegradable polymers such as polylactide-polyglycolide. Depending on the ratio of active agent to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

In certain embodiments, a formulation comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

The composition can be administered in or on a device that allows controlled or sustained release, such as a sponge, biocompatible meshwork, mechanical reservoir, or mechanical implant. Implants (see, e.g., U.S. Pat. No. 5,443,505), devices (see, e.g., U.S. Pat. No. 4,863,457), such as an implantable device, e.g., a mechanical reservoir or an implant or a device comprised of a polymeric composition, are particularly useful for administration of the inventive gene transfer vector. The composition also can be administered in the form of sustained-release formulations (see, e.g., U.S. Pat. No. 5,378,475) comprising, for example, gel foam, hyaluronic acid, gelatin, chondroitin sulfate, a polyphosphoester, such as bis-2-hydroxyethyl-terephthalate (BHET), and/or a polylactic-glycolic acid.

The dose of the vector and/or isolated nucleic acid and/or isolated polypeptide, in the composition administered to the mammal will depend on a number of factors, including the size (mass) of the mammal, the extent of any side-effects, the particular route of administration, and the like. In one embodiment, the inventive method comprises administering a “therapeutically effective amount” of the composition comprising the vector and/or isolated nucleic acid and/or isolated polypeptide, described herein. A “therapeutically effective amount” refers to an amount effective, at dosages and for periods of time as necessary, to achieve a desired therapeutic result. The therapeutically effective amount may vary according to factors such as the extent of the disease or disorder, age, sex, and weight of the individual, and the ability of the gene transfer vector to elicit a desired response in the individual.

The dose of vector in the composition to achieve a particular therapeutic effect typically is administered in units of vector genome copies per cell (gc/cell) or vector genome copies/per kilogram of body weight (gc/kg). One of ordinary skill in the art can readily determine an appropriate vector dose range to treat a patient having a particular disease or disorder, based on these and other factors that are well known in the art. The therapeutically effective amount may be between 1×10¹⁰ genome copies to 1×10¹³ genome copies. The therapeutically effective amount may be between 1×10¹¹ genome copies to 1×10¹⁴ genome copies. The therapeutically effective amount may be between 1×10⁷ genome copies to 1×10¹⁰ genome copies. The therapeutically effective amount may be between 1×10¹⁴ genome copies to 1×10¹⁷ genome copies. Assuming a 70 kg human, the dose ranges may be from 1.4×10⁸ gc/kg to 1.4×10¹¹ gc/kg, 1.4×10⁹ gc/kg to 1.4×10¹² gc/kg, 1.4×10¹⁰ gc/kg to 1.4×10¹³ gc/kg, or 1.4×10¹¹ gc/kg to 1.4×10¹⁴ gc/kg.

The nucleic acids or vectors, or polypeptides, may be administered in dosages of at least about 0.0001 mg/kg to about 1 mg/kg, of at least about 0.001 mg/kg to about 0.5 mg/kg, at least about 0.01 mg/kg to about 0.25 mg/kg or at least about 0.01 mg/kg to about 0.25 mg/kg of body weight, although other dosages may provide beneficial results.

In one embodiment, the composition is administered once to the mammal. It is believed that a single administration of the composition may result in persistent expression in the mammal with minimal side effects. However, in certain cases, it may be appropriate to administer the composition multiple times during a therapeutic period to ensure sufficient exposure of cells to the composition. For example, the composition may be administered to the mammal two or more times (e.g., 2, 3, 4, 5, 6, 6, 8, 9, or 10 or more times) during a therapeutic period.

The present disclosure provides pharmaceutically acceptable compositions which comprise a therapeutically effective amount of vector and/or isolated nucleic acid and/or isolated polypeptide, as described above.

### Routes of Administration, Dosages and Dosage Forms

Administration of, for example, the vectors and/or isolated nucleic acid and/or isolated polypeptide, in accordance with the present disclosure, may be continuous or intermittent, depending, for example, upon the recipient's physiological condition, and other factors known to skilled practitioners. The administration of the vector(s) and/or isolated nucleic acid and/or isolated polypeptide, may be essentially continuous over a preselected period of time or may be in a series of spaced doses. Both local administration, e.g., intracranial, intranasal or intrathecal, and systemic administration, e.g., using viruses that cross the blood-brain barrier, are contemplated. Any route of administration may be employed, e.g., intravenous, intranasal or intrabronchial, direct administration to the lung and intrapleural. In one embodiment, compositions may be delivered to the pleura.

One or more suitable unit dosage forms comprising the vector(s), and/or isolated nucleic acid and/or isolated polypeptide, which may optionally be formulated for sustained release, can be administered by a variety of routes including intracranial, intrathecal, or intranasal, or other means to deliver to the CNS, or oral, or parenteral, including by rectal, buccal, vaginal and sublingual, transdermal, subcutaneous, intravenous, intramuscular, intraperitoneal, intrathoracic, or intrapulmonary routes. The formulations may, where appropriate, be conveniently presented in discrete unit dosage forms and may be prepared by any of the methods well known to pharmacy. Such methods may include the step of bringing into association the vector with liquid carriers, solid matrices, semi-solid carriers, finely divided solid carriers or combinations thereof, and then, if necessary, introducing or shaping the product into the desired delivery system.

The amount of vector(s) and/or isolated nucleic acid and/or isolated polypeptide, administered to achieve a particular outcome will vary depending on various factors including, but not limited to, the genes and promoters chosen, the condition, patient specific parameters, e.g., height, weight and age, and whether prevention or treatment, is to be achieved.

Vectors and/or isolated nucleic acid and/or isolated polypeptide, may conveniently be provided in the form of formulations suitable for administration, e.g., into the brain. A suitable administration format may best be determined by a medical practitioner for each patient individually, according to standard procedures. Suitable pharmaceutically acceptable carriers and their formulation are described in standard formulations treatises, e.g., Remington's Pharmaceuticals Sciences. By “pharmaceutically acceptable” it is meant a carrier, diluent, excipient, and/or salt that is compatible with the other ingredients of the formulation, and not deleterious to the recipient thereof.

Vectors and/or isolated nucleic acid and/or isolated polypeptide, may be formulated in solution at neutral pH, for example, about pH 6.5 to about pH 8.5, or from about pH 7 to 8, with an excipient to bring the solution to about isotonicity, for example, 4.5% mannitol or 0.9% sodium chloride, pH buffered with art-known buffer solutions, such as sodium phosphate, that are generally regarded as safe, together with an accepted preservative such as metacresol 0.1% to 0.75%, or from 0.15% to 0.4% metacresol. Obtaining a desired isotonicity can be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is useful for buffers containing sodium ions. If desired, solutions of the above compositions can also be prepared to enhance shelf life and stability. Therapeutically useful compositions can be prepared by mixing the ingredients following generally accepted procedures. For example, the selected components can be mixed to produce a concentrated mixture which may then be adjusted to the final concentration and viscosity by the addition of water and/or a buffer to control pH or an additional solute to control tonicity.

The vectors and/or isolated nucleic acid and/or isolated polypeptide, can be provided in a dosage form containing an amount of a vector effective in one or multiple doses. For viral vectors, the effective dose may be in the range of at least about 10⁷ viral particles, e.g., about 10⁹ viral particles, or about 10¹¹ viral particles. The number of viral particles added may be up to 10¹⁴. For example, when a viral expression vector is employed, about 10⁸ to about 10⁶⁰ gc of viral vector can be administered as nucleic acid or as a packaged virion. In some embodiments, about 10⁹ to about 10¹⁵ copies of viral vector, e.g., per 0.5 to 10 mL, can be administered as nucleic acid or as a packaged virion. Alternatively, the nucleic acids, polypeptides or vectors, can be administered in dosages of at least about 0.0001 mg/kg to about 1 mg/kg, of at least about 0.001 mg/kg to about 0.5 mg/kg, at least about 0.01 mg/kg to about 0.25 mg/kg or at least about 0.01 mg/kg to about 0.25 mg/kg of body weight, although other dosages may provide beneficial results. The amount administered will vary depending on various factors including, but not limited to, the nucleic acid or vector chosen for administration, the disease, the weight, the physical condition, the health, and/or the age of the mammal. Such factors can be readily determined by the clinician employing animal models or other test systems that are available in the art. As noted, the exact dose to be administered is determined by the attending clinician but may be in 1 mL phosphate buffered saline. For delivery of plasmid DNA alone, or plasmid DNA or RNA, e.g., in a complex with other macromolecules, the amount of DNA to be administered will be an amount which results in a beneficial effect to the recipient. For example, from 0.0001 to 1 mg or more, e.g., up to 1 g, in individual or divided doses, e.g., from 0.001 to 0.5 mg, or 0.01 to 0.1 mg, of DNA or RNA can be administered.

For example, when a viral expression vector is employed, about 10⁸ to about 10⁶ gc of viral vector can be administered as nucleic acid or as a packaged virion. In some embodiments, about 10⁹ to about 10¹⁵ copies of viral vector, e.g., per 0.5 to 10 mL, can be administered as nucleic acid or as a packaged virion. Alternatively, the nucleic acids, polypeptides or vectors, can be administered in dosages of at least about 0.0001 mg/kg to about 1 mg/kg, of at least about 0.001 mg/kg to about 0.5 mg/kg, at least about 0.01 mg/kg to about 0.25 mg/kg or at least about 0.01 mg/kg to about 0.25 mg/kg of body weight, although other dosages may provide beneficial results.

In one embodiment, administration may be by intracranial, intraventricular, intracisternal, lumbar, intrahepatic, intratracheal or intrabronchial injection or infusion using an appropriate catheter or needle. A variety of catheters may be used to achieve delivery, as is known in the art. For example, a variety of general-purpose catheters, as well as modified catheters, suitable for use in the present disclosure are available from commercial suppliers. Also, where delivery is achieved by injection directly into a specific region of the brain or lung, a number of approaches can be used to introduce a catheter into that region, as is known in the art.

By way of illustration, liposomes and other lipid-containing gene delivery complexes can be used to deliver one or more transgenes. The principles of the preparation and use of such complexes for gene delivery have been described in the art (see, e.g., Ledley, (1995); Miller et al., (1995); Chonn et al., (1995); Schofield et al., (1995); Brigham et al., (1993)).

### Subjects

The subject may be any animal, including a human. human and non-human animals. Non-human animals include all vertebrates, e.g., mammals and non-mammals, such as non-human primates, sheep, dogs, cats, cows, horses, chickens, amphibians, and reptiles, although mammals, such as non-human primates, sheep, dogs, cats, cows and horses may be the subject. The subject may also be livestock such as, cattle, swine, sheep, poultry, and horses, or pets, such as dogs and cats.

In one embodiment, subjects include human subjects. The subject is generally diagnosed with the condition by skilled artisans, such as a medical practitioner.

The methods of the disclosure described herein can be employed for subjects of any species, gender, age, ethnic population, or genotype. Accordingly, the term subject includes males and females, and it includes elderly, elderly-to-adult transition age subjects adults, adult-to-pre-adult transition age subjects, and pre-adults, including adolescents, childrens, and infants.

Examples of human ethnic populations include Caucasians, Asians, Hispanics, Africans, African Americans, Native Americans, Semites, and Pacific Islanders. The methods of the disclosure may be more appropriate for some ethnic populations such as Caucasians, especially northern European populations, as well as Asian populations.

The term subject also includes subjects of any genotype or phenotype as long as they are in need of the disclosure, as described above. In addition, the subject can have the genotype or phenotype for any hair color, eye color, skin color or any combination thereof.

The term subject includes a subject of any body height, body weight, or any organ or body part size or shape.

### Exemplary Nucleotide Analogs

The isolated nucleic acid sequences may include one or more nucleotide analogs, e.g., having modifications to the base, e.g., nucleobases including but not limited to 1,5-dimethyluracil, 1-methyluracil, 2-amino-6-hydroxyaminopurine, 2-aminopurine, 3-methyluracil, 5-(hydroxymethyl)cytosine, 5-bromouracil, 5-carboxycytosine, 5-fluoroorotic acid, 5-fluorouracil, 5-formylcytosine, 8-azaadenine, 8-azaguanine, N⁶-hydroxyadenine, allopurinol, hypoxanthine, or thiouracil, modifications of the sugar group or modifications of the phosphate group. In one embodiment, at least one strand of the ds mRNA molecule includes, but is not limited to, 1-methyladenosine, 2-methylthio-N⁶-hydroxynorvalyl carbamoyladenosine, 2-methyladenosine, 2-O-ribosylphosphate adenosine, N⁶-methyl-N⁶-threonylcarbamoyladenosine, N⁶-acetyladenosine, N⁶-glycinylcarbamoyladenosine, N⁶-isopentenyladenosine, N⁶-methyladenosine, N⁶-threonylcarbamoyladenosine, N⁶, N⁶-dimethyladenosine, N N⁶-(cis-hydroxyisopentenyl)adenosine, N⁶-hydroxynorvalylcarbamoyladenosine, 1,2-O-dimethyladenosine, N⁶,2-O-dimethyladenosine, 2-O-methyladenosine, N⁶, N⁶,O-2-trimethyladenosine, 2-methylthio-N⁶-(cis-hydroxyisopentenyl) adenosine, 2-methylthio-N⁶-methyladenosine, 2-methylthio-N⁶-isopentenyladenosine, 2-methylthio-N⁶-threonyl carbamoyladenosine, 2-thiocytidine, 3-methylcytidine, N⁴-acetylcytidine, 5-formylcytidine, N⁴-methylcytidine, 5-methylcytidine, 5-hydroxymethylcytidine, lysidine, N⁴-acetyl-2-O-methylcytidine, 5-formyl-2-O-methylcytidine, 5,2-O-dimethylcytidine, 2-O-methylcytidine, N⁴,2-O-dimethylcytidine, N⁴, N⁴,2-O-trimethylcytidine, 1-methylguanosine, N²,7-dimethylguanosine, N²-methylguanosine, 2-O-ribosylphosphate guanosine, 7-methylguanosine, under modified hydroxywybutosine, 7-aminomethyl-7-deazaguanosine, 7-cyano-7-deazaguanosine, N², N²-dimethylguanosine, 4-demethylwyosine, epoxyqueuosine, hydroxywybutosine, isowyosine, N²,7,2-O-trimethylguanosine, N²,2-O-dimethylguanosine, 1,2-O-dimethylguanosine, 2-O-methylguanosine, N² N22,2-O-trimethylguanosine, N2,N2,7-trimethylguanosine, peroxywybutosine, galactosyl-queuosine, mannosyl-queuosine, queuosine, archaeosine, wybutosine, methylwyosine, wyosine, 2-thiouridine, 3-(3-amino-3-carboxypropyl)uridine, 3-methyluridine, 4-thiouridine, 5-methyl-2-thiouridine, 5-methylaminomethyluridine, 5-carboxymethyluridine, 5-carboxymethylaminomethyluridine, 5-hydroxyuridine, 5-methyluridine, 5-taurinomethyluridine, 5-carbamoylmethyluridine, 5-(carboxyhydroxymethyl)uridine methyl ester, dihydrouridine, 5-methyldihydrouridine, 5-methylaminomethyl-2-thiouridine, 5-(carboxyhydroxymethyl)uridine, 5-(isopentenylaminomethyl)uridine, 5-(isopentenylaminomethyl)-2-thiouridine, 3,2-O-dimethyluridine, 5-carboxymethylaminomethyl-2-O-methyluridine, 5-carbamoylmethyl-2-O-methyluridine, 5-methoxycarbonylmethyl-2-O-methyluridine, 5-(isopentenylaminomethyl)-2-O-methyluridine, 5,2-O-dimethyluridine, 2-O-methyluridine, 2-thio-2-O-methyluridine, uridine 5-oxyacetic acid, 5-methoxycarbonylmethyluridine, uridine 5-oxyacetic acid methyl ester, 5-methoxyuridine, 5-aminomethyl-2-thiouridine, 5-carboxymethylaminomethyl-2-thiouridine, 5-methylaminomethyl-2-selenouridine, 5-methoxycarbonylmethyl-2-thiouridine, 5-taurinomethyl-2-thiouridine, pseudouridine, 1-methyl-3-(3-amino-3-carboxypropyl)pseudouridine, 1-methylpseudouridine, 3-methylpseudouridine, 2-O-methylpseudouridine, inosine, 1-methylinosine, 1,2-O-dimethylinosine and 2-O-methylinosine, or any combination thereof.

In one embodiment, the isolated nucleic acid sequence includes, but is not limited to, cladribine, acyclovir, 2′,3′-dideoxyinosine; 9-β-D-ribofuranosyladenine; .beta.-arabinofuranosylcytosine; arabinosylcytosine; 4-amino-5-fluoro-1-[(2R,5S)-2-(hydroxymethyl)-1,3-oxathiolan-5-yl]-1,2-di-hydropyrimidin-2-one; 2′,3′-dideoxy-3′-thiacytidine; 2′-3′-dideoxycytidine; {(1S,4R)-4-[2-amino-6-(cyclopropylamino)-9H-purin-9-yl]cyclopent-2-en-1-yl}methanol; 2-Amino-9-[(1S,3R,4S)-4-hydroxy-3-(hydroxymethyl)-2-methylidenecyclopenty-1]-6,9-dihydro-3H-purin-6-one; 2′-3′-didehydro-2′-3′-dideoxythymidine; 1-(2-deoxy-.beta.-L-erythro-pentofuranosyl)-5-methylpyrimidine-2,4(1H,3H)-dione; 1-[(2R,4S,5S)-4-azido-5-(hydroxymethyl)oxolan-2-yl]-5-methylpyrimi-dine-2,4-dione; 1-[(2R,4S,5R)-4-hydroxy-5-(hydroxymethyl)oxolan-2-yl]-5-iodo-1,2,3,4-tetr-ahydropyrimidine-2,4-dione; 1-[4-hydroxy-5-(hydroxymethyl)oxolan-2-yl]-5-(trifluoromethyl) pyrimidine-2,4-dione; 5-Fluoro-2′-deoxycytidine; 5-Fluorodeoxycytidine; Floxuridine (5-Fluoro-1-[4-hydroxy-5-(hydroxymethyl)tetrahydrofuran-2-yl]-1H-pyrimidi-ne-2,4-dione); 4-amino-1-(2-deoxy-2,2-difluoro-β-D-erythro-pentofuranosyl)pyrimidin-2(1H)-one; or 2′,2′-difluoro-2′-deoxycytidine; (8R)-3-(2-deoxy-β-D-erythro-pentofuranosyl)-3,4,7,8-tetrahydroimidaz-o[4,5-d][1,3]diazepin-8-ol, or any combination thereof.

In one embodiment, a strand of the ds mRNA may include analogs such as 2′-O-methyl-substituted RNA, locked nucleic acid (LNA) or BNA (Bridged Nucleic Acid), morpholino, or peptide nucleic acid (PNA), or any combination thereof.

In one embodiment, nucleotide analogs include phosphorothioate nucleotides or deazapurine nucleotides and other nucleotide analogs.

In one embodiment, the isolated nucleic acid sequence can include a modified nucleotide selected from a deoxyribonucleotide, a dideoxyribonucleotide, an acyclonucleotide, a 3′-deoxyadenosine (cordycepin), a 3′-azido-3′-deoxythymidine (AZT), a 2′,3′-dideoxyinosine (ddI), a 2′,3′-dideoxy-3′-thiacytidine (3TC), a 2′,3′-didehydro-2′,3′-dideoxythymidine (d4T), a monophosphate nucleotide of 3′-azido-3′-deoxythymidine (AZT), a 2′,3′-dideoxy-3′-thiacytidine (3TC) and a monophosphate nucleotide of 2′,3′-didehydro-2′,3′-dideoxythymidine (d4T), a 4-thiouracil, a 5-bromouracil, a 5-iodouracil, a 5-(3-aminoallyl)-uracil, a 2′-O-alkyl ribonucleotide, a 2′-O-methyl ribonucleotide, a 2′-amino ribonucleotide, a 2′-fluoro ribonucleotide, or a locked nucleic acid; or any combination thereof.

In one embodiment, the nucleotide modification includes 2′ modifications, e.g., 2′ F on pyrimidines or 2′ H or 2′ OMe on purines. In one embodiment, the nucleotide modification includes a phosphate backbone modification selected from a phosphonate, a phosphorothioate, a phosphotriester; a morpholino nucleic acid; or a peptide nucleic acid (PNA).

Sugar modifications in the strand(s) include, but are not limited to, replacing the heteroatoms at the 2′ and 3′ carbons with hydrogen, another heteroatom or an alkyl group; replacing the H's at the 2′ carbon with a heteroatom or alkyl group; replacing the 2′ and 3′ carbons with a heteroatom, most commonly S or O; removing the 2′ and/or 3′ carbons to generate acyclic sugars; replacing the 4′-OH with N, S, or an alkyl group; adding alkyl groups to the 4′-carbon; replacing the 5′-hydroxyl with N or a phosphonate, or interconversion of both the sugar stereochemistry (D vs. L) and anomeric configuration (a vs. p).

### Exemplary Therapeutic Agents

Genetic approaches to alter WDFY3/Alfy expression include but are not limited to ectopic overexpression of WDFY3/Alfy or a portion thereof, as well as the introduction of a single nucleic acid change that represents a coding variant of the gene that encodes WDFY3/Alfy, e.g., a A to G mutation that encodes Iso3032Val change, or expression of AS2 or a portion thereof.

WDFY3-AS2 sequence (this contains the full AS2 sequence):

(SEQ ID NO: 3)
CTACTGAGCCGGCCGCAGAAATTGCAGCCGCTCAGCTTCTACCCCCTCCTGCCTTTCCTTCCTCTTT
CCTTACTTCCTTCCCTTCCCTCGGCTTCCCGCTCTTGCCTCACTCTCAGCGGCTGCCTTCGCCCCTG
TCTGCAGACAGCGCCGCTGGATGCTCCCAGCTGGACTTCAACCCCACTCCTCTCAGTCCCTCTCCCC
ACTGCCTTCCAGACGCGCCTCTTCCCCGCCCCGCGCCCCTCTCTCCTCTCCCACCCCTGCCCCTCT
CCGCGGCGCTCACCCTCCTCAGTCCCAGTTTCTGAAAGGACTCAGCTGAGAAAGGACAACTGGGTT
CCGCTTTCCTTAACCCTACACCCTTTAGCTGGATGCTGTCAGAGGCGATGGAGAGAAACGCAAAGGCTA
CTAGACGCAACAATAGAATTACCATATTGTTTTTCCTGGTTTGAAAGGACCAGATGGAAAGAAACTGG
GCAAGCCAATGAAAGTCTCTGGGGATCATGGGAATTGAGTGTCTATGAAAACCATATTCCAGACAAG
AATATAGTCTAAGGACACAGCAAGAGGCAACATTTTGGAAGCAGAGGGCAACTCTCACTGGAAACCA
AATCTCCTGGCACCTTGATCTTGGATTTTACAGCCTCCAGAACTGATAGCTGGGAGGCATTACATAGT
CATGTGCGAGGTCAAAACTGAGTCACCACCACAACCAAGTTCCAGCTGGCAAGAAGAGGAAAAATAA
CACAGAGGAGTTATGACCAGTGTTGTAAGGATCTGGCCTAGAAATGGCATGCATCTCTTCCACTAATA
TCCTATTGGTAAAAATGTAGTCACATGGGCACACCACCTATCTGCATGGAAGGCTGGGAAATGTTGA
TCGGTAGGGCAGCCATATGTGTAGGAAGGAGAAAACAGAATTTGGTGGATACCTAGTAGTCTCTGCC
ATACCCTTGACCAGAAATTTTTCTTCTAAAGAATTTAATTCTAAAAGTTATAGACATTCCCAAAGATATA
CCTGCAAGCATCACAGCTTTGTGTATAATTGCAAAAGTTGAAATCAACATGAATGCCCAAAATAGTGA
ATTATTTAAACAGATTATTACCCATATTATGAAGAAATACTCTGCAACTCTTAAAAATAAATGTAATGTG
CCAGGCGCAATGGGATATGCCTGCAGGCCCAGCTACACGGGAGGCCAAGGCAGGAGGATCACTTG
AAATCAAGAGTTCAAGTCTAGCCTAGACAACATAGTGAGACTTGGTCTCTAAAAAAGAAAAAAAAAAA
TAAAACTGATGTAATGAATATATAATTACAACAAAAGAGGTCTAACACTATAGTAGAAAAGTTTGGGGC
ACTCATCCCATTCATAATTATTTTACATTTAAATTACTGTTCCTACTTAAGTGGTGAGCTTGGTAACCAT
TTTCATATCAGGTGATACTGCTGCAGTAATCATAGCTGTTTGAATCAGGAATAATCCCAATCACATTTT
CTCTTAATCGTTTATTATACTGGGCATTGGGGTTGCAAGATAATGCACAGTCCGAGCTCAATACATGT
CTTCATGTACCTGTATGTAAATCCCACTTTTATTCTAAAATTCTTTGAATATTTTGTTTCTTGGAACCCA
ATGATCCTAAGAAAATTATGTACTTTTACCTGAAAATTAAAACCAATATTTATAAAAAGTGTATATGCAG
TATGATACCACTTTTAAAAAAAGATAAATTTATAGAAAAAAGTTTATATGACTGACAACAATGTCTCTGA
GTGGTAGGATTACAGTTAATTTTTATTTTCTTCATTCTACTTATCTTTATTTTCTGAACTTTCTACAATGA
ACATGTTTTATTTTTACTTTTGTTAAAATAAATAACATTTAATTTGACGGAAAATTCTAAAAATCAACATT
TGGTAACTTAAATATTCATTGGCACATGATTCTGTGCCTGTTTCTGGGGGTCATTCCCATGAGAAGAG
CAGGTAGAACTTATGAGAATAGAGCAGTCGAAATATTATAGGTTTCATATTTTACGGCTTTATTGAAAA
CATTGATATATTGAATATTCATAAATTTTAATTTAACTTTCTTTTATAATCCTTAATGTGAGGCTAAGGTA
TAAGGAGAAAGGAGATTGGTAAGGAAGTGTGATACATATTGAATCAGGTGTCAAGGTACCATTTGTG
CCTGGATCTGACCAATAGACCAAGGTAAAAATCTCAAAGAATGAGACTTGTAATGAGAATGCCACAAA
CTTGAATACCTTATGCACAAAACACAAACATTGTTATGCATAATGTACATGAACCCTGAAACAAAGATA
ATGATTTGGACCACCCAACTGAAGTGGCATGTTATGTTTTTGGCATGAACAAAAGAAACAAGAGAGAA
AAAAATCAAAATAATTAAAATAAATTGTTAGAGAAAAGTTACTTTAAAAATAGCTATAACGCTTTGCAAT
TTCAGTAACAGTTCTGCCTCTGATGCAATGTAAAACATGTATTAGACATTCAGGCTCCACAATAAATTT
TAATGAGCACTACAAATTTAGAATATATAAAAATATCAGTAACCTATGAATTCATCAACATAACTCACTT
TTCAGTTAAGTTAGGCTGAGTTGAAATTCTACTAAGTTTTCCCATATTCTTGATAAAGGCTAAATTTGA
AATAATTTATTTAGATGACTAATTGGCAATTTGATAGTAGTTACATTTTATGTAACACTTGATATAATAA
CCCAAATTAATTAGCTCAATATTATCTTCTGTGGTTTATTGATCCAAAAGTACATATTTGTTATGTAAAC
TTATACTATGTAAACTTGAATTAGTTTTTTGCTTCCTTGAATTAATAAAGCCCATTAATGCAAATCTAAG
CCTTCATAGACTTTTCCAATCCTTAGTTAAATATTAAAAACTGAGTATTGTAGTTATTTGCTAGGTATAA
AAGGTGACTCAGAGTAGAGAGTAAGATGTCAGCAAATAGACAATAAGATGTCAGCAAAAGTGCCTCA
AATTTTAACTGCGTCAAGTTAAGGGCATTGTAAATACTGTAGTTTTATGTAGCTTGCAACTTCTACTGA
AATGAGTTTAATCATGTCTTTCACATAGCTGAAGTTTGTGTCAAGATTTAAGCTGATTTTCTCATTOTTA
TCAAAGTCTCACCTGGGGGTAGGGAGGGGTAGGCCTACCTCCCACCTCTATACTATCATTTTAGGAA
CAGACAGGCATAAATCTGTTCATATGGTAGAACACATGATGTTAGAGTATATTTTGTTGAATGCTATG
GAATATTAATATAATTAATTCTAAAATATCACCTAAAATATGTCAACTGAAGATCTGACTGTACTAAATA
TGAAAAATAAAGCAGCACATACTTTC

Exemplary portions of AS2 that may be employed in the methods include sequences in bold and/or in red:

CTACTGAGCCGGCCGCAGAAATTGCAGCCGCTCAGCTTCTACCCCCTCCTGCCTTTCCTTCCTCTTTCCTTACTTCCT
TCCCTTCCCTCGGCTTCCCGCTCTTGCCTCACTCTCAGCGGCTGCCTTCGCCCCTGTCTGCAGACAGCGCCGCTGGAT
GCTCCCAGCTGGACTTCAACCCCACTCCTCTCAGTCCCTCTCCCCACTGCCTTCCAGACGCGCCTCTTCCCCGCCCCG
CGCCCCTCTCTCCTCTCCCACCCCTGCCCCTCTCCGCGGCGCTCACCCTCCTCAGTCCCAGTTTCTGAAAGGACTCAG
CTGAGAAAGGACAACTGGGTTCCGCTTTCCTTAACCCTACACCCTTTAGCTGGATGCTGTCAGAGGCGATGGAGAAAC
GCAAAGGCTACTAGACGCAACAATAGAATTACCATATTGTTTTTCCTGGTTTGAAAGGACCAGATGGAAAGAAACTGG
GCAAGCCAATGAAAGTCTCTGGGGATCATGGGAATTGAGTGTCTATGAAAACCATATTCCAGACAAGAATATAGTCTA
AGGACACAGCAAGAGGCAACATTTTGGAAGCAGAGGGCAACTCTCACTGGAAACCAAATCTCCTGGCACCTTGATCTT
GGATTTTACAGCCTCCAGAACTGATAGCTGGGAGGCATTACATAGTCATGTGCGAGGTCAAAACTGAGTCACCACCAC
AACCAAGTTCCAGCTGGCAAGAAGAGGAAAAATAACACAGAGGAGTTATGACCAGTGTTGTAAGGATCTGGCCTAGAA
ATGGCATGCATCTCTTCCACTAATATCCTATTGGTAAAAATGTAGTCACATGGGCACACCACCTATCTGCATGGAAGG
CTGGGAAATGTTGATCGGTAGGGCAGCCATATGTGTAGGAAGGAGAAAACAGAATTTGGTGGATACCTAGTAGTCTCT
GCCATACCCTTGACCAGAAATTTTTCTTCTAAAGAATTTAATTCTAAAAGTTATAGACATTCCCAAAGATATACCTGC

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

indicates data missing or illegible when filed

In one embodiment, the portion has TCTTTCCTTACTTCCTTCCCTTCCCTCGGCTTCCCGCTCTTGCCTCACTC (SEQ ID NO: 11), or a portion thereof, or a nucleotide sequence with at least 80%, 85%, 88%, 90%, 92%, 94%, 95%, 96%, 97%, 98% or 99% nucleic acid sequence identity thereto. In one embodiment, the portion has TCCCACCCCTGCCCCTCT (SEQ ID NO: 12), or a portion thereof, or a nucleotide sequence with at least 80%, 85%, 88%, 90%, 92%, 94%, 95%, 96%, 97%, 98% or 99% nucleic acid sequence identity thereto. In one embodiment, the portion has AACTGGGCAAGCCAATGAAAGTCTCTGGGGAT (SEQ ID NO: 13), or a portion thereof, or a nucleotide sequence with at least 80%, 85%, 88%, 90%, 92%, 94%, 95%, 96%, 97%, 98% or 99% nucleic acid sequence identity thereto. In one embodiment, the portion has GGAAGGCTGGGA (SEQ ID NO: 14), or a portion thereof, or a nucleotide sequence with at least 80%, 85%, 88%, 90%, 92%, 94%, 95%, 96%, 97%, 98% or 99% nucleic acid sequence identity thereto.

The invention will be further described by the following non-limiting examples.

### Example 1

### Results

A pooled, fluorescence-activated cell sorting (FACS)-based genome-wide CRISPR knockout screen in primary mouse macrophages identified known and novel regulators of macrophage efferocytosis. Genome-wide forward genetic screens have the capacity to examine a biological process in an unbiased manner and allow for novel discoveries. The proper cell types for a genome wide CRISPR screen of macrophage efferocytosis were determined. Human monocytic cell lines, including U937 and THP-1, can be differentiated to macrophage-like cells, which have previously been used for genome-wide screening (Sedlyarov et al., 2018; Haney et al., 2018). Yet, it was confirmed that U937 and THP-1 derived macrophages were not a proper model for screening of efferocytosis as monocytic cell line-derived macrophages showed poor efferocytosis capacity. Specifically, upon up to 24 hours of AC incubation, only less than 1-3% of either U937-derived or THP-1-derived macrophages were able to engulf ACs. The results highlight the importance of using physiologically relevant primary macrophages for screening of efferocytosis regulators.

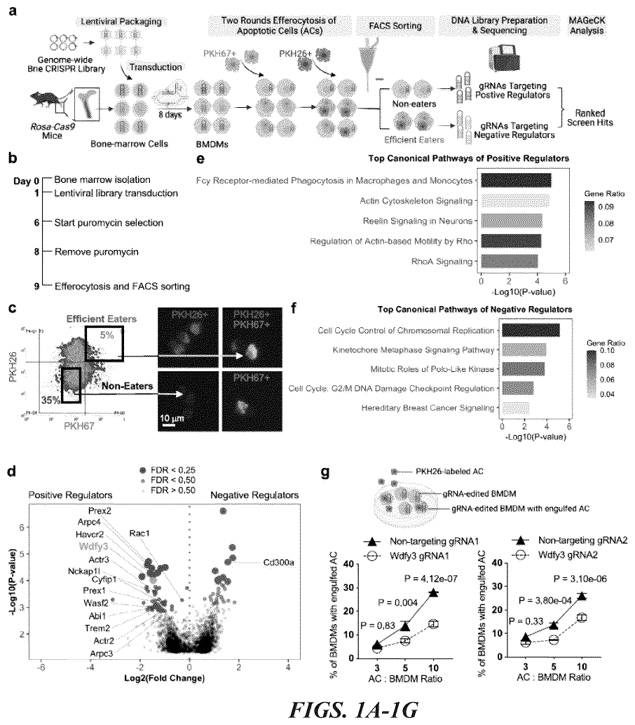

The Rosa26-Cas9 knock-in mice constitutively expressing Cas9 endonuclease (Platt et al., 2014) was leveraged and a workflow for CRISPR gene editing in primary BMDMs established. Specifically, lentiviral gRNA libraries were transduced to isolated bone marrow (BM) cells, which were then differentiated to BMDMs using L cell-conditioned media that provide macrophage colony-stimulating factor (M-CSF) for macrophage differentiation. As illustrated in FIG. 1A and FIG. 1B, for each replicate, 400-500 million BM cells were isolated and seeded. The lentiviral Brie library (Doench et al., 2016) (Addgene 73633) including 78,637 gRNAs targeting 19,674 mouse genes and 1000 non-targeting control gRNAs was transduced on day 1 with a low Multiplicity of Infection (MOI) to ensure that majority of the BM cells integrate one viral particle for gene editing of a single gene. 48 hours after transduction, puromycin was applied to select BM cells with successful lentiviral integration.

The success of the screening relies on the effective enrichment of macrophages with high vs. low efferocytosis capacity. Since efferocytosis is a binary event, to facilitate an effective separation and enrichment, two rounds of efferocytosis were sequentially performed. Specifically, human Jurkat cells (about 10 μm in diameter), an acute T cell leukemia cell line routinely used for in vitro efferocytosis assays, were treated with staurosporine to induce apoptosis, then labeled with fluorescent linkers, PKH67 (Ex/Em: 490/502 nm) or PKH26 (Ex/Em: 551/567 nm), that stains cell membrane. BMDMs were first incubated with PKH67-labeled ACs at a ratio of 5:1 for AC: BMDM and allowed for efferocytosis. After 45 minutes, the unbound PKH67-labeled ACs were washed away and BMDMs were cultured for 2 hours without ACs to allow degradation of the engulfed cargo. Next, BMDMs were fed with PKH26-labeled ACs also at a ratio of 5:1. After 90 minutes, unbound ACs were washed away and BMDMs were collected for flow cytometry sorting. Longer time was allowed for the second round in order to enrich BMDMs that engulf a second AC. Sorting separated the BMDMs that engulfed both PKH67⁺ and PKH26⁺ ACs, i.e., the efficient eaters (about 5%), and BMDMs that did not engulf any ACs, i.e., the non-eaters (FIG. 1C). Two independent replicates were performed. For each replicate, efferocytosis was performed in about 80 million BMDMs on day 9. After sorting, about 3 million efficient eaters were obtained and about 16 million non-eaters. 40 million BMDMs were collected on day 9 without performing efferocytosis, i.e., the input samples.

The sorted non-eaters, efficient eaters, and the input samples for each of the two replicates were sequenced and MAGeCK analysis performed (Li et al., 2014; Li et al., 2015; Chen et al., 2018; Wang et al., 2019) to identify the top hits. Three comparisons were analyzed: input vs. non-eaters, input vs. efficient eaters, non-eaters vs. efficient eaters. The comparison of input vs. non-eaters will likely identify positive regulators whose knockout impairs efferocytosis, while the comparison of input vs. efficient eaters will identify negative regulators whose knockout enhances efferocytosis. The comparison of non-eaters vs. efficient eaters likely further improves the power to identify enriched gRNAs. As expected, the analysis comparing non-eaters vs. efficient eaters was able to identify more known regulators (FIG. 1D). Non-targeting gRNAs did not show enrichment in either sample.

The non-eaters are expected to be enriched for gRNAs targeting positive regulators essential for efferocytosis, i.e., knockout would impair efferocytosis. Indeed, many genes involved in actin polymerization that is known to be essential for phagocytic cup formation, including Rac1, four members of the five-subunit SCAR/WAVE complex (Nckap1l, Wasf2, Abi1, Cyfip1) and five members of the seven-subunit ARP2/3 complex (Actr2, Actr3, Arpc3, and Arpc4) (FIG. 1D) were identified. Pathway analysis was performed using Ingenuity Pathway Analysis (IPA). The top-ranked positive regulators (negative score <0.002, 163 genes) were enriched for pathways including Fcγ Receptor-mediated Phagocytosis in Macrophages and Monocytes, Actin Cytoskeleton Signaling etc. (FIG. 1E), supporting the screening performance in identifying well-known positive regulators. The results also show that many, but not all, genes involved in actin cytoskeleton remodeling and general phagocytosis are among the most highly ranked screen hits.

Using high-content imaging analysis, Arpc4 (top-2 ranked) and Nckap1l (top-14 ranked) were selectively validated using the gRNAs from the original screening library. gRNAs targeting Arpc4 or Nckap1l led to about 50% reduction in the efferocytosis of PKH26-labeled ACs by BMDM. Hacvr2, also known as TIM3, is one of the PtdSer-specific receptors involved in AC recognition and efferocytosis (Zhao et al., 2017). Hacvr2 was ranked at top-7 and was also validated with about 30% reduction in efferocytosis capacity.

The efficient eaters are expected to be enriched for gRNAs targeting negative regulators, i.e., knockout would enhance efferocytosis. Efferocytosis needs to be tightly controlled and there are very few known negative regulators. While this manuscript is being prepared, the top-2 ranked hit for negative regulators, Cd300a (FIG. 1D), was identified as a novel negative regulator (Nakahashi-Oda et al., 2021). Specifically, the binding of an AC with Cd300a and the activation of downstream signaling suppresses efferocytosis by myeloid cells, thus the blockage of Cd300a enhanced efferocytosis (Nakahashi-Oda et al., 2021). The results in BMDM were validated using a gRNA targeting Cd300a. Pathway analysis of top-ranked negative regulators (positive score <0.001 for a total of 96 genes) implies that genes involved in cell cycle control and chromosomal replication were enriched for top hits for negative regulators (FIG. 1F).

The screen has revealed many top-ranked hits that promise to inform novel biology and warrant further validation and functional interrogation. Among the top hits for positive regulators, Wdfy3 is top-10 ranked but not previously implicated in the regulation of efferocytosis or phagocytosis, nor identified in previous screens in nonmammalian cells or using other substrates. Using two individual gRNAs, one from the Brie library and one designed independently, and quantitative imaging analysis, knockout of Wdfy3 in BMDMs led to impaired efferocytosis of PKH26-labeled ACs (FIG. 1G) without affecting BMDM viability. The defects were more significant when BMDMs were challenged with higher AC to BMDM ratio, i.e., a condition mimicking high burden efferocytosis.

Altogether, a CRISPR screen for regulators of efferocytosis, a complex functional phenotype, was established in primary macrophages at genome-wide coverage. Moreover, using this screen a novel regulator, Wdfy3, was identified.

WDFY3 deficiency led to impaired uptake, as opposed to binding, of apoptotic cells due to defective actin depolymerization. WDFY3 encodes a highly conserved, large 400 kDa protein with 3526 amino acids. Similarly to mouse (Dragich et al., 2016), WDFY3 mRNA is the most abundantly expressed in the brain, and in multiple brain cell types. Among immune cells, WDFY3 mRNA expression is abundant in myeloid cells, including macrophages, neutrophils, and monocytes, but not T cells.

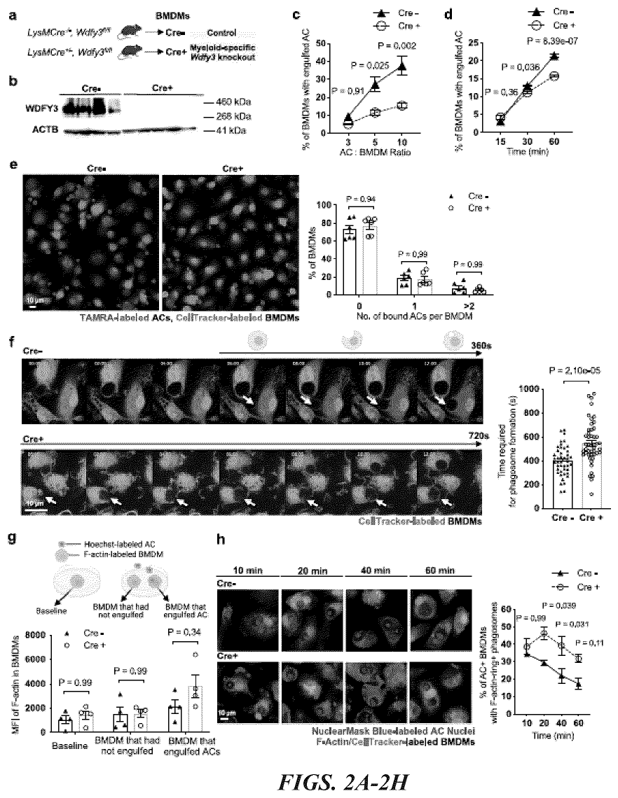

To further validate the role of Wdfy3 knockout in efferocytosis ex vivo, Wdfy3^{fl/fl} mice created by insertion of two loxP sites flanking exon 5 on a 129/SvEv×C57BL/6 background (generated by the Ai Yamamoto lab (Dragich et al., 2016), were obtained. Myeloid-specific Wdfy3 null mice were generated by breeding Wdfy3^{fl/fl} mice with LysMCre mice (JAX 004781), i.e., LysMCre^{+/-}Wdfy3^{fl/fl} mice (Cre⁺) while using LysMCre^{-/-}Wdfy3^{fl/fl} littermates (Cre⁻) as the controls (as illustrated in FIG. 2A). Efficient knockout was confirmed by western blot of WDFY3 in BMDMs from the Cre⁺ mice (FIG. 2B). Although global deletion of Wdfy3 led to perinatal lethality (Dragich et al., 2016), myeloid-specific loss of Wdfy3 did not affect body weight or organ weight, including that of heart, liver, and spleen. Moreover, the mice did not show changes in circulating levels of neutrophils and monocytes, confirming that myelopoiesis was not affected.

Flow cytometry was used to quantify the percentage of BMDMs with engulfed ACs labeled with Hoechst. With lower AC:BMDM ratio or at relatively early time points, efferocytosis of Cre⁻ and Cre⁺ BMDMs appeared similar (FIG. 2C, D). The defects were more significant with a high ratio of AC:BMDM that resembles high burden efferocytosis (FIG. 2C). Consistently, with an AC: BMDM ratio at 5:1, the defective efferocytosis in Cre⁺ BMDMs was the most significant at later time points (FIG. 2D), also supporting more pronounced defects over prolonged periods of challenges. These effects were independent of sex.

Efferocytosis involves the finding, recognition and binding, uptake, and finally the degradation of the engulfed cargos (Boada-Romero et al., 2020; Doran et al., 2019). The screen identifies regulators essential for the binding and/or uptake of ACs but is not designed to identify genes involved solely in cargo degradation, if the binding or uptake of ACs is unaffected. The screen will also not identify genes solely responsible for the chemotactic cues termed find-me signals because the pooled design masks the defective secretion by a small subset of edited cells.

With this in mind, the molecular steps regulated by WDFY3 were determined. First it was determined if Wdfy3 knockout affected binding and/or uptake during efferocytosis. TAMRA-labeled apoptotic murine thymocytes were incubated with CellTracker-labeled BMDMs pretreated with cytochalasin D that prevents actin polymerization thus the uptake of ACs at a 5:1 AC:BMDM ratio (Wang et al., 2017; Perry et al., 2019; Moon et al., 2020). Following incubation, unbound ACs were washed away and BMDMs were fixed and imaged. The numbers of TAMRA-labeled ACs bound with each BMDM were counted and the percentage of BMDMs with none, one, or two and more bound ACs was quantified for Cre⁻ and Cre⁺ BMDMs. The results support that Wdfy3 knockout did not affect the ability of BMDMs to bind ACs (FIG. 2E), suggesting that the uptake, as opposed to binding, of ACs was impaired due to Wdfy3 knockout.

Time-lapse live-cell imaging confirmed that the time required for complete internalization of ACs was longer in Wdfy3 knockout BMDM compared with control (FIG. 2F), suggesting delayed phagosome formation. Phagosome formation during phagocytosis of large particles requires the coordination of actin polymerization and depolymerization, permitting the continual restructuring of the actin cytoskeleton (Schlam et al., 2015). Complete internalization of the cargo is synchronized with actin depolymerization, allowing subsequent phagosome maturation (Lerm et al., 2007). Thus, it was asked if Wdfy3 knockout affects actin polymerization and/or depolymerization. BMDMs were labeled with siR-actin, a fluorogenic, cell-permeable probe based on an F-actin binding natural product jasplakinolide and determined F-actin levels at baseline and upon efferocytosis of Hoechst-labeled ACs by flow cytometry. F-actin signals at baseline were similar between Cre⁻ and Cre⁺ BMDMs (FIG. 2G, left panel). Upon efferocytosis, BMDMs that had not engulfed ACs also showed comparable F-actin levels between Cre⁻ and Cre⁺ BMDMs (FIG. 2G, middle panel). Yet, in BMDMs that engulfed ACs, Wdfy3 knockout BMDMs showed a trend of higher F-actin signals (FIG. 2G, right panel, P=0.34). Although not statistically significant, the observed trend led to the hypothesis that potential defects in actin disassembly exist in Wdfy3 knockout BMDMs. Indeed, in Wdfy3 knockout BMDMs that had successfully internalized an AC, it was observed that many engulfed ACs were surrounded by F-actin rings (FIG. 2H). Confirming the subjective observations, the percentage of BMDMs with F-actin surrounded cargos over all BMDMs that had engulfed ACs was greater in Cre⁺ vs. Cre⁻ BMDMs (FIG. 2H). In Cre⁻ control BMDMs, the percentage of BMDMs with F-actin surrounded cargos was the highest at 10 minutes after adding ACs and then decreased over time (FIG. 2H). Yet, in Cre⁺ BMDMs, the percentage further increased and peaked at 20 minutes after adding ACs and remain greater than the percentage in Cre⁻ BMDMs (FIG. 2H), supporting defective actin depolymerization.

Thus, defective actin depolymerization in Wdfy3 knockout macrophages led to impaired uptake and delayed phagosome formation during efferocytosis. The defects were specific to efferocytosis of ACs because the phagocytosis of other substrates, including polystyrene beads of different sizes (4 μm and 10 μm), sheep red blood cells (RBCs) that were untreated, stressed by heat treatment, or IgG-opsonized, zymosan particles (500 nm), was not impaired in Wdfy3 knockout BMDM. Consistently, previous screens using the above-mentioned substrates in U937monocytic line derived macrophages (Haney et al., 2018), or using cancer cells in J774 macrophages (Kamber et al., 2021) did not uncover Wdfy3 as a hit. Thus, a novel regulator specifically required for the uptake of ACs during efferocytosis was confirmed.

How macrophages involve different molecular machinery to regulate the engulfment of various cargos remains largely unknown. Recent studies revealed that the engulfment of larger cargos (e.g., 5 μm beads) requires phosphoinositide 3-kinase (PI3K)-mediated PtdIns(3,4,5)₃ production and PtdIns(3,4,5)₃-dependent recruitment of GTPase-activating proteins (GAPs) that inactivates Rho GTPases Rac/Cdc42, therefore allowing cycling of F-actin assembly and disassembly (Schlam et al., 2015). This mechanism may also be required for the engulfment of ACs (about 10 μm for Jurkat cells). Indeed, PI3K inhibitor, LY294002, markedly reduced the uptake of ACs in both control and Wdfy3 knockout BMDMs, implicating that WDFY3 was not required for PI3K activation. It was reasoned that if WDFY3 is downstream of PI3K-mediated F-actin disassembly, with PI3K inhibitor treatment, knockout of Wdfy3 should not further impair AC uptake. In fact, with PI3K inhibition, Wdfy3 knockout BMDMs showed lower AC uptake compared with control BMDMs, supporting that WDFY3 affects AC uptake at least partly through PI3K and GAP-independent mechanisms. As expected, when PI3K is inhibited, uptake of 10 μm beads was comparable between Wdfy3 knockout and control BMDMs, suggesting that WDFY3-mediated regulatory mechanisms are not required for beads engulfment. Consistently, the percentage of BMDMs with F-actin surrounded beads was also comparable between Wdfy3 knockout and control BMDMs, in sharp contrast to the higher percentage of F-actin ring surrounded engulfed ACs in Wdfy3 knockout BMDMs compared with control BMDMs (FIG. 2H). Several studies have reported that macrophages more effectively engulf rigid cargos than soft cargos (Beningo & Wang, 2002; Jaumouille et al., 2019), likely because soft cargos deform and thus requiring stronger force generation (Jaumouille & Waterman, 2020). It was speculated that WDFY3-mediated F-actin dynamics is essential for the engulfment of the more challenging cargos, such as ACs, while dispensable for the uptake of the less challenging cargos, such as cargos with smaller size or higher rigidity. The observations account for the differential requirement for WDFY3 during efferocytosis and pave the way for further interrogating the complex molecular mechanisms employed by macrophages in cargo-specific phagocytosis.

The role of Wdfy3 in macrophage efferocytosis in Wdfy3^{fl/fl} mice generated by the Knock-Out Mouse Project (KOMP) with two lox p sites flanking exon 8 and maintained on C57BL/6N background (Orosco et al., 2019) was validated. Breeding to LysMCre mice led to efficient knockout of Wdfy3 though a small amount of residual protein remained detectable. Consistently, impaired uptake of ACs in Cre⁺ BMDMs was observed, further confirming that the role of Wdfy3 knockout in macrophage efferocytosis is independent of the genetic strain or specific gene inactivating mutation of the mouse models.

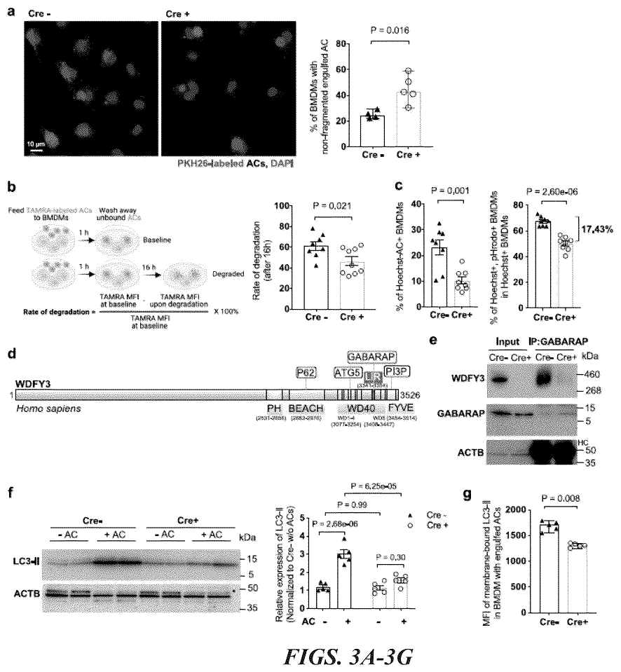

WDFY3 deficiency led to impaired degradation of engulfed ACs. Sustained accumulation of periphagosomal F-actin prevents efficient phagosome-lysosome fusion (Lerm et al., 2007). Thus, it was reasoned that defective actin depolymerization may impair the degradation of the engulfed cargos. To test the hypothesis, the degradation of the engulfed ACs by Cre⁺ and Cre⁻ BMDMs was determined. BMDMs were first incubated with PKH26-labeled ACs for efferocytosis. After 60 minutes of incubation, unbound ACs were washed away and BMDMs were returned to the incubator for 3 hours to allow degradation of the engulfed cargos. BMDMs were then fixed and imaged. The percentage of AC⁺ BMDMs that showed non-fragmented PKH26 staining implicating impaired degradation was counted. Indeed, the percentage of BMDMs with non-fragmented ACs was greater in Cre⁺ vs. Cre⁻ BMDMs (FIG. 3A), confirming impaired degradation in Wdfy3-deficient BMDMs. To further validate the degradation defects using an alternative approach via flow cytometry-based quantification (Perry et al., 2019), BMDMs were incubated with ACs labeled by TAMRA, a dye that labels peptides and proteins, for efferocytosis and quantified the degradation of TAMRA signals 16 hours post-efferocytosis. Consistently, Wdfy3 knockout led to decreased rates of corpse degradation (FIG. 3B).

To dissect if the impaired degradation in Wdfy3 knockout BMDMs is also linked to impaired lysosomal acidification, ACs were dual-labeled with Hoechst that stains DNA and is pH-insensitive, and pHrodo-Red that is pH-sensitive and shows fluorescent signals only under an acidified environment in the phagolysosome. Wdfy3 knockout BMDMs showed a lower efferocytosis of Hoechst-labeled ACs (FIG. 3C, left panel), consistent with the results in FIG. 2C. For BMDMs with engulfed Hoechst⁺ ACs, the percentage of pHrodo⁺/Hoechst⁺ BMDMs in Hoechst⁺BMDMs is lower in Cre⁺ BMDMs vs. Cre⁻ BMDMs, supporting impaired acidification in Wdfy3 knockout BMDMs (FIG. 3C, right panel). Consistent results were observed using peritoneal macrophages (PMs). Thus, WDFY3 is required for both the uptake and the degradation of engulfed ACs during efferocytosis, and Wdfy3 knockout led to impaired acidification of the phagolysosome.

WDFY3 deficiency led to defects in LAP. Next it was asked if the impaired degradation in Wdfy3 knockout BMDMs was merely a consequence of the defects in actin depolymerization during phagosome formation or mediated by other potentially independent mechanisms. First it was considered whether WDFY3 is involved in LC3-associated phagocytosis (LAP), a process by which LC3-II conjugation to phagosomes enables phagosome-lysosome fusion and AC corpse degradation (Martinez et al., 2015; Martinez, 2018; Cunha et al., 2018; Heckmann et al., 2017; Green et al., 2016). The hypothesis is built on the known role of WDFY3 in autophagic clearance of aggregated proteins, i.e., aggrephagy (Filimonenko et al., 2010; Fox et al., 2020). Specifically, The C-terminus of both mouse and human WDFY3 contains several functional domains (as illustrated in FIG. 3d) (Simonsen et al., 2004). Co-immunoprecipitation and colocalization studies indicated that WDFY3 scaffolds a complex containing the p62-positive, ubiquitinated, aggregation-prone protein and the core autophagy proteins ATG5, ATG12, ATG16L1 and LC3/GABARAP (Filimonenko et al., 2010; Lystad et al., 2014). The human ortholog of the yeast Atg8 includes the LC3 family (LC3A, LC3B, LC3B2 and LC3C) and the GABARAP family (GABARAP, GABARAPL1 and GABARAPL2). During aggrephagy, the WD40 repeats of WDFY3 are essential for its colocalization and interaction with ATG539. The ATG5-ATG12 complex is required for an early stage of autophagosome formation, and together with the membrane-bound ATG16L1 facilitate the conjugation of LC3/GABARAP proteins to phosphatidylethanolamine, i.e., LC3 lipidation to form LC3-II, for autophagosome formation (Hanada et al., 2007; Fujita et al., 2008). Recent work using HEK293T cells further revealed that WDFY3 has a conserved LIR (LC3-interacting region) motif in its WD40 region that directly binds to GABARAP, responsible for its recruitment to LC3B during aggrephagy (Lystad et al., 2014).

First it was determined if endogenous WDFY3 interacts with GABARAP in macrophages. Whole-cell lysates from Cre⁻ and Cre⁺ BMDMs were incubated with anti-GABARAP antibodies and were immunoprecipitated using protein A/G agarose beads. WDFY3 can be found in a complex with endogenous GABARAP in Cre⁻ BMDMs, confirming WDFY3 and GABARAP interactions (FIG. 3E). No precipitation was observed in Wdfy3 knockout BMDMs, confirming the specificity of the antibody (FIG. 3E).

It was thus reasoned that WDFY3 interacts with GABARAP, regulating the recruitment and lipidation of LC3 during LAP for subsequent cargo degradation. Consistent with previous literature (Martinez et al., 2011), AC engulfment led to increased LC3-II as determined by western blot (FIG. 3F). The increase was blunted in Wdfy3-deficient BMDMs (FIG. 3F). The results were confirmed using a flow cytometry-based assay (Martinez, 2020). Specifically, BMDMs were incubated with Hoechst-labeled ACs to allow efferocytosis. After removal of unengulfed ACs, BMDMs were collected and treated with digitonin to remove non-membrane bound LC3, and then immunostained for LC3 that is lipidated and membrane bound, i.e., LC3-II. As quantified by flow cytometry, for BMDMs that had engulfed Hoechst-labeled ACs, Wdfy3 knockout BMDMs had lower membrane-bound LC3-II (FIG. 3G), supporting impaired LC3 lipidation.

Taken together, WDFY3 regulates LAP-mediated degradation of engulfed ACs through interacting with GABARAP and facilitating LC3 lipidation and the subsequent phagolysosomal degradation.

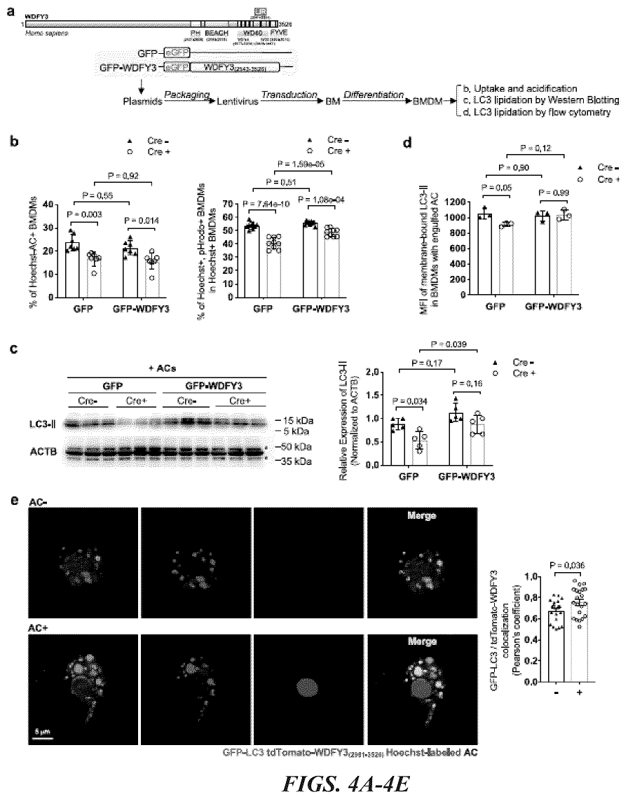

A C-terminus fragment of WDFY3 is sufficient for regulating degradation yet the full-length protein is required for the AC uptake during efferocytosis. It has previously been shown that a 1000 amino acid C-terminus fragment, that contains the PH-BEACH, WD40, LIR, and FYVE domains of WDFY3 or the D. melanogaster ortholog, Bluecheese, was sufficient to enhance the degradation of aggregated proteins in otherwise wildtype cells (Filimonenko et al., 2010; Eenjes et al., 2016). Therefore, it was asked if this fragment was sufficient to regulate uptake and/or degradation during efferocytosis. Lentiviral transduction was used to express C-terminal WDFY3 in both Cre⁻ and Cre⁺BM cells that were then differentiated to BMDMs (as illustrated in FIG. 4A). Although expression of C-terminal WDFY3₍₂₅₄₃₋₃₅₂₆₎ did not rescue the defective uptake in Cre⁺ BMDMs (FIG. 4B), it was sufficient to partly rescue the defects in the acidification of the engulfed ACs (FIG. 4B). Mechanistically, expression of C-terminal WDFY3 restored LC3 lipidation as quantified by both western blot (FIG. 4C) and flow cytometry (FIG. 4D). Thus, Wdfy3 knockout led to two major defects affecting lysosomal acidification: (1) WDFY3 deficiency disrupted its direct interaction with GABARAP/LC3 complex that facilitates LC3 lipidation and phagosome-lysosome fusion and subsequent acidification; (2) Knockout of Wdfy3 led to defects in F-actin disassembly, which is expected to delay the subsequent phagosome-lysosome fusion and lysosomal acidification. Therefore, though the C-WDFY3 completely rescued LC3 lipidation, the acidification defects were partially rescued, likely because C-WDFY3 was not sufficient to rescue defects in uptake.

Demonstrating WDFY3 localization and LC3 phagosome recruitment using microscopy is important to strengthen further the conclusion on the role of WDFY in LAP. Because of the lack of a reliable antibody for immunofluorescence staining of WDFY3 and the technical challenge to package the full-length WDFY3 cDNA (which is 10.8 kb thus preventing effective transfection or transduction), tdTomato was fused to C-terminal WDFY3 and transfected the construct via electroporation to BMDMs from GFP-LC3 transgenic mice and imaged the cells with or without AC engulfment. As shown in FIG. 4E, C-WDFY3 showed cytoplasmic localization. Without AC engulfment, BMDMs from GFP-LC3 mice showed basal levels of LC3 punta. With AC engulfment, LC3 punta showed association with the phagosome. LC3 and C-WDFY3 colocalization also increased in AC-engulfed BMDMs vs. BMDMs without AC uptake. Thus, the imaging data are consistent with western blot and flow cytometry data, supporting the role of WDFY3 in interacting with LC3 complex and facilitating LC3 lipidation in LAP.

Wdfy3 knockout subtly affects the transcriptome of BMDMs without affecting macrophage differentiation. To gain an unbiased view of how Wdfy3 knockout affects the transcriptomic signature of macrophages, RNA-seq was performed in Cre and Cre⁺ BMDMs (n=4 male mice). First it was confirmed that many receptors responsible for efferocytosis and phagocytosis, including Fcgr1, Fcgr2b, Fcgr3, Mertk, Timd4, and many macrophage marker genes, were expressed at similar levels between Cre⁻ and Cre⁺ BMDMs. Using a FDR-adjusted P value <0.05 and absolute fold-change >1.5, only a small number of genes were identified as differentially expressed (DE) between Cre⁻ and Cre⁺ BMDMs, i.e., 20 genes were upregulated while 32 genes were downregulated in Cre⁺ vs. Cre⁻ BMDMs.

It was reasoned that modest changes in the expression of genes belonging to the same pathway may imply functional impact. Gene-set enrichment analysis (GSEA) was performed to determine which gene sets or pathways were enriched in upregulated or downregulated genes due to Wdfy3 knockout. The upregulated genes in Cre⁺ BMDM were enriched for Human Reactome Pathway terms, IL-4 and IL-13 Signaling and Collagen Formation, and GO Biological Process term, Regulation of Chemotaxis. The downregulated genes in Cre⁺ BMDM were enriched for Human Reactome Pathway term, Peroxisomal Lipid Metabolism, and Gene Ontology (GO) Biological Process term, Fatty Acid Catabolic Process. Overall, no clear proinflammatory or anti-inflammatory gene signatures were identified in Cre⁺ BMDMs.

Thus, it was confirmed that: (1) Despite the profound role of WDFY3 in AC uptake and degradation, the observed transcriptomic modifications by Wdfy3 knockout were modest; (2) Wdfy3 knockout did not affect macrophage maturation, as macrophage marker genes were not differentially expressed. Further it was confirmed that the percentage of F4/80⁺ macrophages in BMDMs and PMs was comparable between Cre⁻ and Cre⁺ mice. Population doubling during BMDM differentiation was not different between Cre⁻ and Cre⁺ mice, supporting comparable differentiation and proliferation capacity.

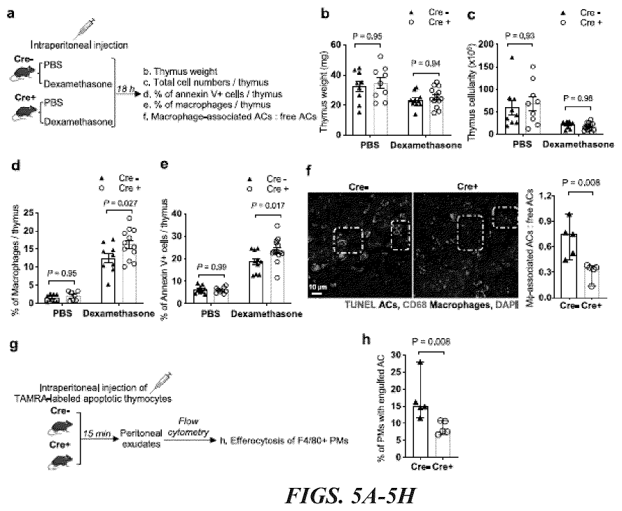

Mice with myeloid Wdfy3 knockout show impaired efferocytosis in vivo. To determine if Wdfy3 knockout affects efferocytosis in vivo, two in vivo efferocytosis assays were performed in Cre⁻ and Cre⁺ mice as illustrated in FIG. 5a (thymus efferocytosis) and FIG. 5g (PM efferocytosis).
For thymus efferocytosis (FIG. 5A), Cre⁻ and Cre⁺ mice were treated with dexamethasone that induces apoptosis of thymocytes, using PBS as the control. 18 hours after injection, thymi were isolated and weights were measured. The total number of cells per thymus was determined by dissociating one lobe of the thymus to count the cell number and then normalized to the weight of both lobes of the thymus. The dissociated cells were stained for Annexin V, a marker of apoptosis, and macrophage marker F4/80, for quantification by flow cytometry. As expected, in dexamethasone-treated mice, coupled processes of thymocyte apoptosis and phagocytic clearance of dead cells led to reduced thymus weight (FIG. 5b) and the total number of cells per thymus (FIG. 5C), accompanied by increased macrophage infiltration (FIG. 5D) and a higher percentage of Annexin V⁺ cells in the thymus (FIG. 5E). A significant change in thymus weight or the total number of cells in Cre⁺ mice compared to Cre⁻ controls was not observed, yet myeloid Wdfy3 knock-out led to an increased percentage of Annexin V⁺ cells (FIG. 5E), implying impaired efferocytic clearance of apoptotic thymocytes (FIG. 5E). Note that the impaired efferocytic clearance in Cre⁺ mice was unlikely to be caused by reduced macrophage availability because the percentage of macrophages per thymus was not lower in Cre⁺ mice vs. Cre⁻ mice treated with either PBS or dexamethasone (FIG. 5D). Induction of apoptosis by dexamethasone was equivalent between Cre⁻ and Cre⁺ mice for both thymocytes and BMDMs), ensuring that the increased percentage of Annexin V⁺ cells in Cre⁺ mice was due to impaired clearance, not altered induction of apoptosis, To assess efferocytosis in the thymus in situ, thymus sections were labeled and fluorescently imaged for TUNEL⁺ cells (ACs) that were either associated with CD68⁺ macrophages as a result of efferocytosis, or not associated with macrophages, i.e., free ACs, indicating inefficient efferocytic clearance. The ratio of macrophage associated vs. free ACs was significantly lower in Cre⁺ mice, further supporting impaired efferocytosis in Wdfy3 knockout mice (FIG. 5F).

For PM efferocytosis (FIG. 5G), TAMRA-labeled apoptotic thymocytes were injected intraperitoneally into Cre⁻ and Cre⁺ mice. 15 minutes after injection, peritoneal exudate was collected and stained for F4/80 to identify macrophages. The percentage of TAMRA⁺ PMs was quantified by flowcytometry. Consistent with thymus efferocytosis, the percentage of TAMRA⁺ PMs was significantly lower in Cre⁺ mice (FIG. 5H), supporting reduced AC efferocytosis by PMs in vivo.

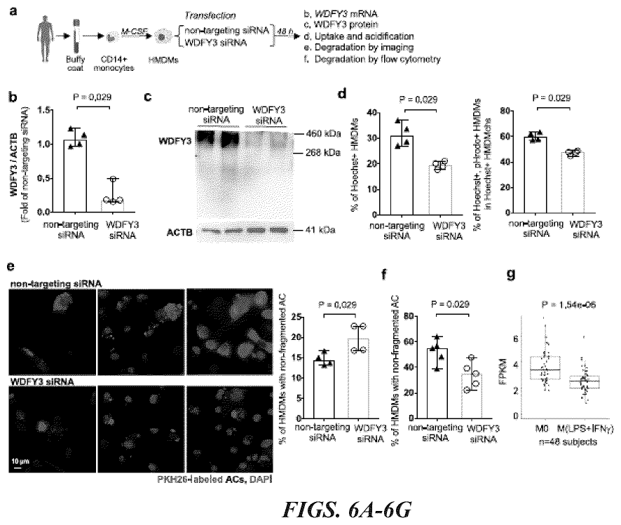

WDFY3 is required for efferocytosis in human macrophages. Further it was confirmed that in human macrophages, knockdown of WDFY3 by transfection of small interfering RNA (siRNA) led to impaired uptake and degradation of engulfed ACs during efferocytosis (FIG. 6A-F). Human CD14⁺ monocytes were isolated from buffy coats of three independent subjects and differentiated to macrophages (human monocyte-derived macrophages, HMDMs) using M-CSF. On day 5, non-targeting control siRNA pool or WDFY3-targeting siRNA pool were transfected using Lipofectamine RNAiMAX (FIG. 6A). At 48 hours post-transfection, efficient knockdown of WDFY3 was confirmed at both mRNA (FIG. 6B) and protein levels (FIG. 6C). Efferocytosis of human Jurkat cells labeled by both Hoechst and pHrodo was performed. Consistent with the results in murine macrophages, both uptake and acidification (FIG. 6D) of ACs were impaired in HMDMs with siRNA mediated WDFY3 knockdown. The percentage of HMDMs with non-fragmented ACs was greater with WDFY3 knockdown (FIG. 6E). Flow cytometry analysis further confirmed lower degradation rate of TAMRA signals in HMDMs with WDFY3 knockdown, supporting impaired degradation of TAMRA-labeled ACs (FIG. 6F). In querying previously published RNA-seq data for HMDMs either unstimulated (MO) or treated with LPS and IFNγ for 18-20 hours (M1-like) (Fan et al., 2020), M1 stimulation reduced WDFY3 mRNA expression (FIG. 6G). Thus, reducedWDFY3 during inflammation and subsequently impaired WDFY3-mediated macrophage efferocytosis may contribute to impaired efferocytotic clearance of ACs in vitro and in vivo, exacerbating inflammation.

### Discussion

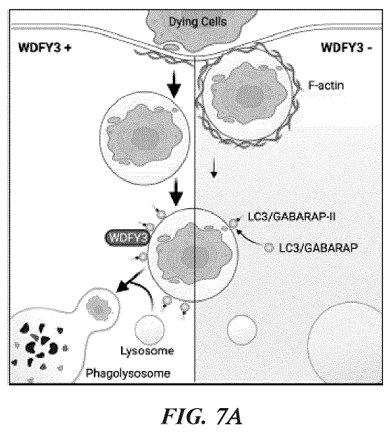

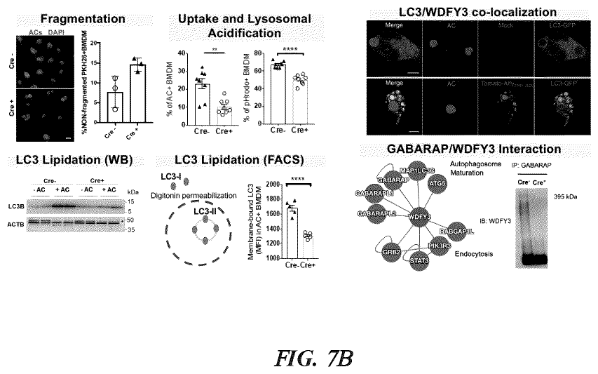

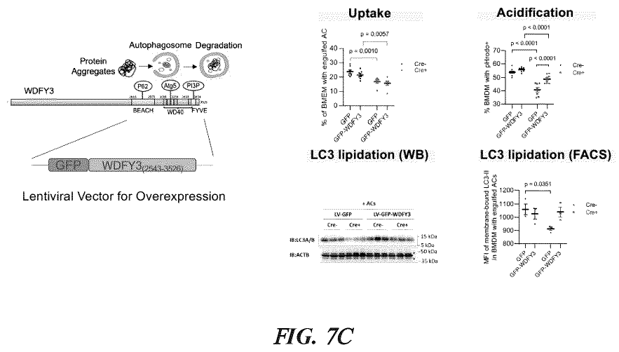

A genome wide CRISPR knockout screen in primary macrophages was developed. By focusing on efferocytosis, a complex macrophage functional phenotype, it was illustrated the versatility of pooled screens and provided an effective approach for genome wide CRISPR screening in primary macrophages derived from Cas9 transgenic mice. Many known genes regulating efferocytosis and general phagocytosis were identified, illuminating the important genes essential for the uptake of ACs during efferocytosis. WDFY3 was uncovered an validated as a novel regulator specifically regulating the phagocytosis of dying cells, but not other substrates, using orthogonal assays in vitro and in vivo. Mechanistically, WDFY3 deficiency led to impaired phagosome formation due to defects in actin depolymerization. Further it was revealed that WDFY3 directly interacts with GABARAP, one of the seven members of the LC3/GABARAP protein family, to facilitate LC3 lipidation and the efficient degradation of the engulfed cargo (FIG. 7 for a schematic summary). Further, WDFY3 expression was suppressed by inflammatory stimulation. Thus, WDFY3 regulates multiple steps during efferocytosis. Targeting WDFY3 may have therapeutic implications for diseases related to defective efferocytosis.

Unexpectedly a novel role of WDFY3 in LAP was uncovered. The detailed molecular mechanisms require further investigation. The FYVE domain binds to phosphatidylinositol 3-monophosphate (PI3P). PI3P is produced during both autophagosome and phagosome formation and is required for the recruitment of autophagic machinery for downstream fusion with lysosomes. It is therefore plausible that during efferocytosis, WDFY3 is recruited through its known PI3P binding domain and acts as a scaffold that bridges ACs and autophagic machinery to regulate phagosome-lysosome fusion and lysosomal degradation of the engulfed cargos. Other questions remain unanswered, e.g., whether identical or different functional domains and binding partners have been involved in efferocytosis vs. in WDFY3-mediated aggrephagy; what is the function of N-terminal WDFY3; which molecular domains are required and sufficient for the role of WDFY3 in uptake and/or degradation and what are their protein-protein interaction partners; what are the molecular mechanisms by which WDFY3 regulates F-actin disassembly and how WDFY3-mediated F-actin dynamics specifically regulate AC uptake. Pull-down experiments using specific domains of WDFY3 followed by quantitative proteomic screening, and live-cell imaging of endogenously tagged WDFY3 at baseline and during efferocytosis will further uncover these molecular mechanisms.

WDFY3 was highly expressed in myeloid cells compared with other immune cells. WDFY3 expression in HMDM was reduced by proinflammatory stimulation with LPS and IFNγ. The role of WDFY3-mediated efferocytosis in inflammation resolution and the therapeutic potential to enhance WDFY3 in diseases related to defective efferocytosis warrant further investigation. Indeed, overexpression of C-terminal WDFY3 (WDFY32981-3526) can enhance aggrephagy in neurons as indicated by increased aggregate clearance (Filimonenko et al., 2010; Eenjes et al., 2016), supporting the therapeutic premise to target macrophage WDFY3 to stimulate efferocytosis. Therapeutic activation of WDFY3 may represent a proefferocytotic therapy in atherosclerosis and other diseases related to defective efferocytosis.

The screen has implied many highly ranked, potentially novel regulators of macrophage efferocytosis. Among the top-ranked positive regulators, in addition to Wdfy3, Sh3glb1, Snx24, and Vps33a are annotated in autophagy-related pathways. The generation of PI3P on the phagosomal membrane recruits LC3-conjugation machinery, and abrogation of LC3 lipidation at the membrane impairs phagosome maturation and lysosome-mediated degradation (Boada-Romero et al., 2020; Martinez, 2018). SH3GLB1 activates lipid kinase activity of PIK3C3 during autophagy by associating with the PI3K complex II (PI3KC3-C2) (Takahashi et al., 2007). SNX24 contains a PX domain that mediates specific binding to membranes enriched in PI3P50. VPS33A is required for lysosome fusion with endosomes and autophagosomes (Wartosch et al., 2015). These top screen hits may represent additional novel components of the cellular machinery that regulates efferocytosis. These promising targets and other potential novel regulators as uncovered by the screen have tremendous potential for additional novel discoveries. In the genome-wide screen, a strategy of interpreting the results with a relatively permissive FDR threshold was employed. Secondary screens with an increased number of gRNAs per gene and the number of cells infected per gRNA are expected to further improve the specificity and sensitivity for pooled screens in primary cells (Doench, 2018).

Furthermore, this screening platform can be adapted to screen for phagocytic regulators of distinct substrates, e.g., bacteria and amyloid-O aggregates, for which the engulfment by physiologically relevant primary macrophages will be more informative, and to study gene pairs with epistatic interactions using libraries with multiplexed gRNAs. The platform will facilitate the identification of efferocytosis regulators affecting distinct molecular steps, including recognition and degradation. For example, by applying different selection strategies to separate macrophages with engulfed and acidified cargos from those with engulfed yet non-acidified cargos, genes specifically regulating the intracellular processing and degradation can be systematically interrogated. Further, screening for regulators responsible for efferocytosis of dying cells undergoing different modes of cell death can be studied. Since the number of macrophages required for genome-wide coverage and the required Cas9 transgenic expression makes it impractical for genome-wide pooled screens to be performed in human primary macrophages, screens in primary murine macrophages provide opportunities for physiologically relevant discoveries of novel biology, which can then be validated in human macrophages. The experimental framework also provides a general strategy for systematic identification of genes of interest and uncovering novel regulators of complex macrophage functions, such as lipid uptake and foam cell formation. This genetic platform promises to accelerate clinically relevant, mechanism-based translational research projects in macrophage biology and related human diseases.

In summary, a pooled genome wide CRISPR knockout screen was established in primary macrophages for discoveries of novel regulators of macrophage efferocytosis. The screen has revealed WDFY3 as a regulator of efferocytosis in vitro and in vivo, in the mouse and in human cells. The findings advance the understanding of fundamental mechanisms of efferocytosis regulated by WDFY3. The screen top hits may likely contain additional novel regulators that can be further validated and promise to yield insights into diseases manifested by dysregulated efferocytosis. The innovative screen approaches established in this project are of broad and fundamental value to the community for conducting functional screens of novel regulators of complex macrophage function.

### Methods

### Cell Lines

Cell lines, including Jurkat (lymphocytes, human acute T cell leukemia), THP-1 (monocytes, human acute monocytic leukemia), U937 (monocytes, human histiocytic lymphoma), and L-929 (mouse fibroblasts) were obtained from ATCC and handled according to the instructions provided on the ATCC product sheet.

### Bone-Marrow Isolation and Differentiation to Bone Marrow Derived Macrophages (BMDMs).

Bone-marrow (BM) cells from 8 to 12 weeks old mice were isolated by flushing femurs and tibia with DMEM basal medium using 10 mL syringes with 22 G needles (day 0). The isolated BM cells were cultured at 37° C., 5% CO₂ on non-tissue-culture-treated vessels for 7-10 days in BMDM culture medium containing DMEM supplemented with 10% (vol/vol) heat-inactivated fetal bovine serum (HI-FBS), 20% (vol/vol) L-929 fibroblast conditioned medium, and 2 mM L-Glutamine. During differentiation, the growth medium was replaced with fresh medium 96 hours after seeding and then every 2-3 days. In vitro assays were performed in BMDMs from day 7 to day 10.

### Peritoneal Macrophage (PM) Isolation.

PM isolation buffer (DPBS supplemented with 10% (vol/vol) HI-FBS and 2 mM EDTA) was injected into the peritoneum of 8-12 weeks old mice for a 10-minute incubation. Peritoneal exudates were then collected using 10 mL syringes with 25 G needles and plated on non-tissue-culture treated vessels. Unattached cells were removed 6 hours after plating and the attached cells were used. PMs were maintained in DMEM supplemented with 10% (vol/vol) HI-FBS, 20% (vol/vol) L-929 fibroblast conditioned medium, and 2 mM L-Glutamine for 12-18 hours at 37° C., 5% CO₂ before the indicated assays (Moon et al., 2020).

### Human-Monocyte-Derived Macrophages (HMDMs).

Buffy coats of anonymous, de-identified healthy adult volunteer donors were purchased from the New York Blood Center (NYBC), with informed consent obtained by the NYBC, for isolation of peripheral blood mononuclear cells (PBMCs). Buffy coats were diluted with 1×DPBS supplemented with 2 mM EDTA at a 1:1 ratio, i.e., 8 mL buffy coats were diluted with 8 mL DPBS to a total volume of 16 mL The diluted buffy coats were carefully laid on 9 mL Ficoll-Paque solution, i.e., a 4:3 ratio in 50-mL conical tubes and centrifuged at 400 g for 40 minutes at 20° C. without brake. PBMC layer was transferred and washed with washing buffer (1×DPBS, 2% (vol/vol) HI-FBS, 5 mM EDTA, 20 mM HEPES and 1 mM sodium pyruvate), centrifuged at 500 g for 10 minutes at 4° C. The PBMC pellets were washed again in RPMI-1640 medium containing 20% (vol/vol) HI-FBS. The pellets were then resuspended and cultured in RPMI-1640 medium supplemented with 20% (vol/vol) HI-FBS and 50 ng/mL human macrophage colony-stimulating factor (M-CSF) for 7-10 days (Zhang et al., 2015). The growth medium was replaced with fresh medium 96 hours after seeding and then every 2-3 days.

### THP-1 and U937 Differentiation to Macrophages.

THP-1 human acute monocytic leukemia cell line was obtained from ATCC and grown in suspension in THP-1 culture medium containing RPMI-1640 supplemented with 10% (vol/vol) HI-FBS, 1 mM Sodium Pyruvate, 10 mM HEPES, and 50 μM 2-Mercaptoethanol. THP-1 macrophages were differentiated from THP-1 cells in the above culture media supplemented with 100 nM Phorbol 12-myristate 13-acetate (PMA) for 24 hours at a seeding density of 1×10⁶ cells per well of a 6-well tissue culture plate. PMA-containing media was then removed and replaced with THP-1 culture media for 48 hours culture. The same seeding density was used for U937 differentiation to macrophages with 50 nM PMA for 3 days (Haney et al., 2018).

### Experimental Animals

Animal protocols were approved by the Institutional Animal Care And Use Committee at Columbia University (Protocol Number ACAABN5558). All animals were cared for according to the NIH guidelines. Mice were socially housed in standard cages at 22° C. with 40-60% humidity under a 12-12 hours light-dark cycle with ad libitum access to water and food provided by the mouse barrier facility (PicoLab Rodent Diet 20 5053 and 5058, LabDiet). Rosa26-Cas9 knockin mice were obtained from the Jackson Laboratory (Cat #026179, C57BL/6J) (female mice were used for the CRISPR screen and validation). Wdfy3fl/fl mice were obtained from Dr. Ai Yamamoto's lab (Wdfy3^{fl/fl}: 129/SvEv×C57BL/6 flanking Exon 6) (Dragich et al., 2016) and Dr. Konstantinos Zarbalis's lab (Wdfy3^{fl/fl}: C57BL/6NJ flanking Exon 8). Myeloid-specific Wdfy3 knockout mice were created by crossing LysMCre^{+/-} mice (the Jackson Laboratory, Cat #004781, C57B16 J) with Wdfy3^{fl/fl} mice. LysMCre^{+/-}Wdfy3^{fl/fl} mice (Cre⁺) had myeloid-specific knockout of Wdfy3, while LysMCre^{-/-}Wdfy3^{fl/fl} littermates (Cre⁻) served as controls. The GFP-LC3 mice (Mizushima et al., 2004; Kuma & Mizushima, 2008) were maintained as homozygote (Eenjes et al., 2016) (Background strain C57B16 J). The wild-type mice for thymocyte isolation were obtained from the Jackson Laboratory (Cat #000664, C57BL/6J). Mice were euthanized by CO₂ asphyxiation followed by cervical dislocation. Both male and female mice were used at 8-12 weeks old unless otherwise specified, and experimental and control mice were co-housed.

### Lentiviral Plasmid Construction

The Brie murine CRISPR knockout pooled library in the lentiGuide-Puro backbone was obtained from Addgene (#73663) (Doench et al., 2016). To validate the top screen hits using individual gRNAs, pairs of oligonucleotides with BsmBI-compatible overhangs were separately annealed and cloned into the lentiGuide-Puro vector (Addgene #52963) using standard protocols available via https://www.addgene.org/52963/. To validate the role of Wdfy3 using a separate plasmid platform, gRNA targeting Wdfy3 was selected from the murine Sanger lentiviral CRISPR library (Sigma) and the Wdfy3-targeting lentiviral vector, as well as the nontargeting control vector, were obtained (Sigma). To overexpress C-terminal WDFY3, pLE4-eGFP-WDFY₃₂₅₄₃₋₃₅₂₆ was constructed by inserting Myc-WDFY3₍₂₅₄₃₋₃₅₂₆₎, which was from pcDNA-myc-WDFY₃₂₅₄₃₋₃₅₂₆ provided by Dr. Ai Yamamoto (Filimonenko et al., 2010), into the pLE4 lentiviral backbone (Fang et al., 2018). eGFP was then inserted into the N-terminal of WDFY3 to generate pLE4-eGFP-WDFY3₍₂₅₄₃₋₃₅₂₆₎ to allow the identification of WDFY3-overexpressing population by flow cytometry upon transduction.

### Lentiviral Packaging and Transduction

Lentivirus particles were generated from HEK293T cells (ATCC CRL-3216) by co-transfection of lentiviral vectors with the packaging plasmid psPAX2 (Addgene #12260) and envelope plasmid pMD2G (Addgene #12259) using FuGene 6 transfection reagent (Promega). The medium was changed 16-18 hours after transfection. 24 hours after media change, lentiviral supernatants were harvested and stored at 4° C. Fresh media were fed and lentiviral supernatants were collected again 24 hours later and pooled together with the first harvest. The pooled supernatants were then filtered through 0.45-μm SFCA filters (Corning). Lentiviral particles were further concentrated using Lenti-X concentrator (Takara Bio) following the manufacturer's instructions.

Mouse BM cells were isolated and plated (day 0). On day 1, BM cells were virally transduced in BMDM culture medium supplemented with 10 μg/mL polybrene. On day 2, half of the medium was replenished with fresh BMDM culture medium. On day 6, the transduced cells underwent puromycin selection at 5 μg/mL for 48 hours. On day 9, i.e., 24 hours after removing puromycin, BMDMs were used for efferocytosis assays. The pLE4 lentiviral vector does not have a puromycin-resistant gene, thus no antibiotics selection was performed. For pLE4 lentivector expressing GFP only or GFP-WDFY3, transduction was performed on day 0 and assays were performed on day 8.

### Induction of Apoptosis and Fluorescent Labeling of Apoptotic Cells (ACs)

Apoptotic Jurkat cells were generated by treating Jurkat cells with 5 μg/mL staurosporine in RPMI-1640 medium for 3 hours at a density of 2.5×10⁶ cells/mL at 37° C., 5% CO₂. The method routinely yields greater than 90% Annexin V⁺ apoptotic Jurkat cells. After washing in 1×DPBS, apoptotic Jurkat cells were resuspended at a concentration of 2×10⁷ cells/mL in Diluent C with either PKH67 (green fluorescence) or PKH26 (red fluorescence) per the manufacturer's instruction. After labeling, the cells were rinsed twice with DMEM basal medium containing 10% HI-FBS and immediately used for efferocytosis assay. For labeling with other fluorescent probes, ACs were resuspended at a density of 2.5×10⁶ cells/mL in DMEM basal media and incubated with 20 ng/mL pHrodo red (Life Technologies) and/or Hoechst 33342 solution (20 mM, 1:10,000 dilution, Thermo Scientific) for 30 minutes, or NuclearMask Blue Stain solution (1:2000 dilution, Invitrogen) for 30 minutes. TAMRA staining was applied to ACs at a concentration of 2×10⁷ cells/mL in DMEMbasal medium at 10 μg/mL for 25 minutes.

To isolate mouse thymocytes and induce apoptosis, thymi were dissected from C57BL/6 J mice (about 6-weeks) and were grounded and filtered through 70 μm cell strainer to obtain single-cell suspension. The induction of apoptosis can be initiated by one of the two approaches: (1) Incubating the thymocytes with 50 μM dexamethasone in DMEM at 37° C., 5% CO₂ for 4 hours; (2) UV irradiation (Analytik Jena UVP EL Series Lamps, UVP95020001) for a total of 12 minutes and then incubated for 2.5 hours at 37° C. with 5% CO₂ (Wang et al., 2017) and the percentage of Annexin V⁺ cells that are apoptotic were confirmed to be >90%. Labeling of apoptotic thymocytes was performed as described above for Jurkat ACs.

### Preparation of Sheep Red Blood Cells (RBCs) for Efferocytosis

Sheep red blood cells (RBCs) (Rockland) were obtained. For heat-shock treatment, RBCs were incubated under 56° C. in a water bath for 5 minutes (Chang et al., 2018). For IgG-opsonization, RBCs were incubated with 1 μg/mL anti-RBCs antibodies in DMEM basal medium containing 10% (vol/vol) HIFBS to conjugate with IgG at 37° C., 5% CO₂ for 1.5 hours (Chang et al., 2018). The non-treated, heat-shock treated, or IgG-conjugated RBCs were labeled with PKH67 following the same procedures for the labeling of apoptotic Jurkat cells.

### In Vitro Efferocytosis and Phagocytosis Assays

For imaging-based quantification, macrophages were plated in 96-well plates at a density of 0.3×10⁵ per well. For flow cytometry-based quantification, macrophages (BMDMs, PMs, or HMDMs) were plated in 6-well or 24-well plates at a density of 1.5×10⁶ per well or 0.2×10⁶ per well, respectively. Fluorescently labeled apoptotic cells were co-incubated with macrophages at a 5:1 AC: macrophage ratio for 1 hour (or as described in Figures) at 37° C., 5% CO₂. Macrophages were then washed with 1×DPBS gently to remove unbound targets. For imaging-based quantification, macrophages were fixed with 2% PFA for 30 minutes, rinsed 3 times with 1×DPBS, and counterstained by DAPI. For flow cytometry-based quantification, macrophages were lifted using Cell-Stripper, a non-enzymatic cell dissociation solution, for live-cell analysis. The phagocytosis of beads, RBCs, and zymosan particles by BMDMs was determined upon incubation for 1 hours at the specific ratio or concentration as specified in the respective figures.

To determine how inhibiting PI3K affects macrophage efferocytosis and phagocytosis, BMDMs were pretreated with 10 μM PI3K inhibitor LY294002 for 60 minutes and during efferocytosis. The percentage of AC-engulfed or beads-engulfed BMDMs was quantified by flow cytometry.

### CRISPR-Cas9 Screen for Efferocytosis in BMDMs and Validation

CRISPR-Cas9 screens were performed using the Brie library (Doench et al., 2016). BM cells isolated from Rosa-Cas9 knock in mice were virally transduced at a low multiplicity of infection (MOI) of 0.3 and targeting about 1000-fold coverage of the library. After puromycin selection, BMDMs were dissociated and replated in 10-cm tissue culture plates at a density of 6×10⁶ per plate for two-round efferocytosis. For the 1st round, PKH67-labeled ACs were incubated with BMDMs at a 5:1 ratio for 45 minutes. After removing the unbound ACs, macrophages were rested for 3 hours before the 2^{nd} round, in which PKH26-labeled ACs were incubated with BMDMs at a 5:1 ratio for 90 minutes. Unbound ACs were removed and BMDMs were collected for sorting on BD Influx. The sorted populations were processed individually for genomic DNA extraction using DNeasy Blood and Tissue Kit (Qiagen) and subjected to PCR reactions to amplify the gRNA sequences and generate the libraries. The purified PCR products were sequenced on Illumina NextSeq 500 system to determine gRNA abundance in two independent replicates. The fastq files were processed using count_spacers.py to obtain the gRNA counts (Joung et al., 2017) (refer to the Code Availability statement for code). The gRNA count matrix files were then analyzed using MAGeCK (Model-based Analysis of Genome-wide CRISPR-Cas9 Knockout (Wang et al., 2019), version 0.5.7). MAGeCK (mageck test) uses Robust Rank Aggregation (RRA) for robust identification of CRISPR-screen hits, and outputs the summary results at both sgRNA and gene level as ranked lists of screen hits. Independent validation of top screen hits by individual gRNAs was performed by lentiviral transduction of gRNA in Rosa-Cas9 knock-in BM cells and differentiation to BMDM followed by efferocytosis assays and quantification (Nakahashi-Oda et al., 2021).

Initial validation of top screening hits as shown in FIG. 1G was performed using Nikon Ti-S Automated Inverted Microscope with NIS-Elements High Content Analysis Imaging Software according to the manual. Briefly, nuclei were segmented as primary objects by DAPI images. Cut-off size was optimized to remove improperly segmented cells, such as large debris and apoptotic bodies, from further analyses. Individual cell outlines were obtained by growing all initial nuclei regions simultaneously until they touch or reach the image border, i.e., Watershed. ACs were segmented by PKH26 images. Cut-offs to the size, roundness, and intensity of the signals were optimized to remove auto-fluorescent bodies and cell debris. The count feature was then used to count cells and cells with ACs and calculate the percentage of cells with ACs. The analysis template was provided in the Github repository, which can be used as guidance rather than prescriptive, as differences in staining intensity can affect the effectiveness of segmentation.

### Analysis of Macrophage Capability of Binding

BMDMs were stained with 0.5 μM CellTracker Green CMFDA (5-chloromethylfluorescein diacetate) for 60 minutes. The CellTracker dye freely passes through cell membranes and is well-retained in cells, allowing labeling of cytoplasmic area. BMDMs were then treated with 5 μM cytochalasin D for 30 minutes. Cytochalasin D blocks the assembly and disassembly of actinmonomers, thus preventing internalization of ACs. The treated BMDMs were then incubated with TAMRA-stained apoptotic mouse thymocytes for 30 min at a 5:1 ratio of AC: BMDM at 37° C., 5% CO₂ to allow binding. The unbound ACs were extensively washed with 1×DPBS, BMDMs were fixed with 2% PFA for 30 minutes and washed with 1×DPBS for 3 times, followed by imaging with ImageXpress Micro 4 High-Content Imaging System with a Nikon Plan Apo λ 20×/0.75 objective lens to analyze the percentage of macrophages with bound ACs.

### Time-Lapse Imaging of Phagosome Formation

BMDMs cultured on chambered coverslips with 8 individual wells (ibidi) at a density of 0.12×10⁶ were stained with 0.5 μM CellTracker Green CMFDA Dye (Invitrogen) in DMEM supplemented with 10% (vol/vol) HI-FBS for 60 minutes. The medium was replaced with fresh DMEM containing 10% HI-FBS and apoptotic Jurkat cells were added at a 5:1 AC:BMDM ratio. BMDMs were imaged with Nikon Ti Eclipse inverted microscope for spinning-disk confocal microscopy equipped with a 60×/1.49 Apo TIRF oil immersion lens. Images of the same fields were recorded at 30 s intervals for 20 minutes.

### Visualization and Quantification of F-Actin Dynamics During Efferocytosis of ACs or Phagocytosis of Beads

BMDMs plated on 96-well plates were stained with 0.5 μM CellTracker Green CMFDA Dye (Invitrogen) and 1 μM SiR-actin (Cytoskeleton) for 60 minutes. ACs labeled by NCS-nucleomask blue (Invitrogen) were added to the macrophages to replace the staining medium at a 5:1 AC: macrophage ratio for 1-hour efferocytosis. Macrophage monolayer was then washed with 1×DPBS to remove unbound ACs, fixed with 2% PFA for 30 minutes and washed with 1×DPBS for 3 times, and imaged by ImageXpress Micro4 high-content microscopy (Molecular Device) with a Nikon Plan Apo λ 40×/0.95 objective lens. The percentage of macrophage with bright F-actin ring, as an indicator of F-actin polymerization, was quantified.

To quantify F-actin intensity by flow cytometry, BMDMs plated on 6-well non-tissue culture plates were incubated with Hoechst-labeled ACs for 1 hour. Unbound ACs were washed away and BMDMs were collected and fixed by 2% PFA for staining with 1 μM siR-actin in washing buffer (1×DPBS, 2% (vol/vol) HI-FBS, 5 mM EDTA, 20 mM HEPES and 1 mM sodium pyruvate). siR-actin-labeled F-actin levels were quantified as the mean fluorescence intensity (MFI) of siR-actin in BMDMs with or without engulfment of ACs.

To assess F-actin dynamics during phagocytosis of beads, BMDMs were stained with 1 μM CellMask-Green Actin Tracking Dye (Invitrogen) for 30 minutes. 10 μm red fluorescent polystyrene beads (Invitrogen) were added at a 5:1 ratio to macrophage for 1-hour efferocytosis. The same procedures were followed as above to image and quantify the percentage of macrophage with F-actin rings.

### Analysis of Fragmentation of Engulfed AC Components by Imaging

PKH26-labeled ACs were added to BMDMs or HMDMs and incubated for 45 minutes. Unengulfed ACs were removed by vigorous rinsing with 1×DPBS. After being cultured for an additional 3 hours, the macrophages were fixed with 2% PFA and counterstained with DAPI. Images were captured using ImageXpress Micro4 high-content microscopy (Molecular Device) with a Nikon Plan Apo λ 40×/0.95 objective lens. The percentage of macrophages containing non-fragmented AC-derived fluorescence, which is a measure of AC corpse degradation, was quantified (Wang et al., 2017).

### Analysis of Degradation of Engulfed AC by Flow Cytometry

TAMRA-labeled ACs were added to CellTracker Green stained BMDMs or HMDMs and incubated for 45 minutes. Unengulfed ACs were removed by 1×DPBS wash. Macrophages were harvested for flow cytometry to quantify the MFI of TAMRA⁺CellTracker⁺ population at baseline and 16 hours after efferocytosis ended. The rate of degradation was calculated as (MFI of TAMRA at 0 hours-MFI of TAMRA at 16 hours)/MFI of TAMRA at baseline X 100%.

### Membrane-Bound LC3 Detection by Flow Cytometry

BMDMs were incubated with Hoechst-labeled ACs at a 5:1 AC:BMDM ratio at 37° C., 5% CO₂ for 1-hour efferocytosis. Unbounded ACs were washed away. BMDMs were collected and resuspended in 300 μL cold DPBS with 20 μg/mL digitonin and incubated on ice for 10 minutes to permeabilize cells and allow non-membrane bound LC3 to be removed from cells. Permeabilized BMDMs were then centrifuged for 5 minutes at 750 g, followed by incubation with anti-LC3A/B-FITC antibody or anti-LC3A/B-PE antibody diluted in cold washing buffer (1×DPBS, 2% (vol/vol) HI-FBS, 5 mM EDTA, 20 mM HEPES and 1 mM sodium pyruvate) for 15 minutes on ice to stain the membrane-bound lipidated LC3-II within the cells. After staining, macrophages were washed with 1 mL cold washing buffer and were centrifuged for 5 minutes at 750 g. Cell pellets were resuspended in washing buffer and acquired on a flow cytometer (Martinez et al., 2011).

### Membrane-Bound LC3 Detection by Immunoblotting

UV-induced apoptotic thymocytes were added to BMDMs cultured in 6-well plate at a ratio of 5:1. After incubating for 1.5 hours, BMDMs were washed 3 times with DPBS and harvested with CellStripper. Cells from each well were lysed with 70 μL RIPA lysis buffer (Millipore) supplemented with protease inhibitor cocktail (Roche) and phosphatase inhibitor cocktail (Roche) for 30 minutes on ice. Lysates were then centrifuged at 12,000 g for 10 minutes and supernatant were transferred to a fresh tube. Protein concentration was quantified using Pierce BCA protein assay kit (ThermoFisher). Equal amounts of protein were mixed with 4× Bolt LDS sample buffer and 10× reducing reagent dithiothreitol (Novex Bolt Sample Reducing Agent, 10×). Samples were heated for 10 min at 60° C. and centrifuge at 12,000 g for 30 seconds before loading to a 16% Tris-glycine gel. Proteins were then electro-transferred to a 0.45 μm (or 0.2 μm) PVDF membrane (Thermo Scientific). After blocking with 5% milk, the membrane was incubated with rabbit anti-LC3B primary antibody (ab48394, Abcam) overnight at 4° C. The membrane was then washed for 3 times in TBST and incubated with HRP-conjugated goat anti-rabbit IgG (1:5000 dilution) for 1 hour at room temperature. After the final wash to remove unbound antibodies, the protein expression was detected by SuperSignal™ West Pico PLUS Chemiluminescent Substrate (Thermo Scientific) and imaged using ChemiDoc Imaging System (Bio-rad). Band intensity was quantified using the software ImageJ.

### Immunoblotting of WDFY3

Macrophages cultured on 6-well plate were harvested and cells from one well were lysed in 70 μL RIPA lysis buffer (Millipore) supplemented with protease inhibitor cocktail (Roche) and phosphatase inhibitor cocktail (Roche). Protein concentration was quantified using Pierce BCA protein assay kit (Thermo Fisher). Equal amount of protein were mixed with 5×SDS sample buffer [5% (vol/vol) β-Mercaptoethanol, 0.02% (vol/vol) Bromophenol blue, 30% (vol/vol) Glycerol, 10% (vol/vol) Sodium dodecyl sulfate, 250 mM Tris-Cl, pH 6.8)] and loaded onto a 3-8% Tris-Acetate NuPage gel and then electro-transferred to a 0.45 μm (or 0.2 μm) PVDF membrane (Thermo Scientific). After blocking with 5% milk, the membrane was incubated with rabbit anti-WDFY3 primary antibody (Ai Yamamoto lab (Fox et al., 2020)) overnight at 4° C. The membrane was then washed for 3 times in TBST and incubated with HRP-conjugated goat anti-rabbit IgG (1:5000 dilution) for 1 hour at room temperature. After the final wash to remove unbound antibodies, the protein expression was detected by SuperSignal™ West Pico PLUS Chemiluminescent Substrate (Thermo Scientific) and imaged using ChemiDoc Imaging System (Bio-rad). The membranes were then blocked with 5% milk for 30 min followed by incubating with HRP-conjugated antibody to blot ACTB (β-actin) for 1 hour at room temperature. After washing with TBST for 3 times, the membranes were imaged using ChemiDoc Imaging System (Bio-rad). Band intensity was quantified using the software image J.

### GABARAP Immunoprecipitation

Around 20×10⁶ BMDMs were harvested from two 10-cm dishes and lysed in 600 μL RIPA lysis buffer (Millipore) supplemented with protease inhibitor cocktail (Roche) on ice for 30 minutes. The lysates were centrifuge at 12,000 g for 10 minutes. 450 μL supernatant was taken out and transferred into a fresh pre-chilled tube, followed by measuring the protein concentration with Pierce BCA protein assay kit (Thermo Fisher). For input sample preparation, about 5% of total protein was aliquoted to a separate tube and mixed with 4× Bolt LDS sample buffer as well as 10× reducing reagent for GABARAP blot. About 5% of total protein were aliquoted and mixed with 5×SDS sample buffer for WDFY3 blot. 10 μg of the input samples were loading to the gel.

For Pull-down sample preparation, 250 μg total cell lysates were incubated with 8 μg anti-GABARAP antibodies (Abcam, ab191888) in 500 μL RIPA buffer overnight at 4° C. 100 μL protein A/G agarose beads (Thermo Scientific Pierce) were centrifuged at 600 g and washed with RIPA buffer for 3 times and were then added to the antibody and lysates mixture for another 1-2 hours at 4° C. After incubation, the mixture was centrifuged at 600 g for 1 minute. After removing supernatant, beads were washed with 1 mLRIPA buffer for 3 times. Each time remained 100 μL at bottom. For eluting the immunoprecipitants from the antibody and beads, the sample after the final wash were separated to two tubes. One tube containing 40 μL beads was then mixed with 2× Bolt LDS sample buffer with 2× reducing reagent for GABARAP blotting. The remaining 40 μL beads were incubates with 2×SDS sample buffer for WDFY3 blotting. Samples were heated at 60° C. for 10 minutes and centrifuge at 800 g to elute the proteins. Fifteen microliters of the pulldown samples were then subjected to 3-8% Tris-glycine gel for immunoblotting analysis.

Live-Cell Imaging of GFP-LC3. tdTomato-WDFY3. And Quantification of Colocalization

BM cells were isolated from GFP-LC3 transgenic mice and then differentiated to BMDMs. At day 5, BMDMs were lifted up and transfected with pDEST-tdTomato-WDFY3₍₂₉₈₁₋₃₅₂₆₎ (Filimonenko et al., 2010), a plasmid construct with tdTomato-fused to the N-terminal of the C-terminal WDFY3₍₂₉₈₁₋₃₅₂₆₎, via electroporation using P3 primary cells kit (Lonza, V4XP-3032). After electroporation, cells were seeded into chambered coverslip (ibidi, 80826) to continue differentiation. At day 8 or day 9, apoptotic thymocytes were labeled with Hoechst for 30 minutes at 1:10,000, and then fed to BMDMs for 15 minutes and washed with DPBS for 8-10 times. Note that a shorter incubation time and extensive washing step were used to ensure minimal amount of unengulfed ACs remaining on the coverslip in order to perform live-cell imaging using a Nikon spinning-disk confocal microscope with Plan Apo λ 100×/0.95 oil objective. Quantification of colocalization was performed using the ImageJ JACoP (Just Another Colocalization Plugin59 version 2.0). Pearson's coefficient is a commonly used colocalization indicators that measures the strength of a linear relationship between two variables (Moreau et al., 2015). Using JACoP, Pearson's coefficient of GFP and tdTomato signals was calculated for each cell.

### RNA-Sequencing and Gene Set Enrichment Analysis

Total RNAs were extracted from day 8 BMDMs (9-10 weeks old male mice: 4 Cre⁻ and 4 Cre⁺) using the Quick-RNA miniprep plus kit (Zymo). With a minimum of 300 ng input RNA, strand-specific, poly(A)+libraries were prepared and sequenced at 20 million 100-bp paired-end reads per sample. Raw sequencing reads were mapped to the mouse genome version GRCm39 (M27) using Salmon (Patro et al., 2017) (version 1.5.1) to obtain transcript abundance counts. MultiQC was used to generate quality control reports based on Salmon read mapping results. The transcript-level count information was summarized to the gene level using tximport62 (version 1.20.0). Differential expression was assessed using DESeq263 (version 1.34.0). Genes with an absolute fold-change >1.5 and false discovery rate (FDR)-adjusted P value <0.05 were considered as differentially expressed (DE). The output of DESeq2 were scored and ranked based on P value and shrunken log 2 fold-change by apeglm (Zhu et al., 2019) using ranking metrics −log 10 P value multiplied by the sign of log-transformed fold-change (Reimand et al., 2019). The ranked gene list was then used for Gene Set Enrichment Analysis (GSEA)66 (version 4.2.0) with the weighted statistics to identify the gene sets overrepresented at the top or bottom of the ranked list using the Human Reactome Pathway (the most actively updated general-purpose public database of human pathways) and the Gene Ontology Biological Process annotation (the most commonly used resource for pathway enrichment analysis) within the Molecular Signatures Database. Only ontologies with more than 15 genes and less than 200 genes were considered. g:Orth was used to translate gene identifiers from mouse to human based on the information retrieved from the Ensembl database (Raudvere et al., 2019).

### Ingenuity Pathway Analysis

Ingenuity pathway analysis (IPA) software using build-in scientific literature-based database (according to IPA Ingenuity Web Site, www.ingenuity.com) was used to identify canonical pathways, overrepresented in top-scored CRISPR screen hits.

### Quantitative RT-PCR

Total RNA was extracted using Quick-RNA Miniprep Kit (Zymo) and cDNA was synthesized using High-Capacity cDNA Reverse Transcription Kit (Applied Biosystems) as per the manufacturer's instructions. To measure gene expression, quantitative RT-PCR was performed using POWERUP SYBR GreenMasterMix by QuantStudio™ 7 Flex Real-Time PCR System (Applied Biosystem, 4485701). ΔΔCT method was used to analyze the relative levels of each transcript normalized to human ACTB.

### In Vivo Thymus Efferocytosis Assay

Cre⁺ and Cre⁻ mice of 8-12 weeks old were injected intraperitoneally with 200 μL PBS or 200 μL PBS containing 250 μg dexamethasone. Dexamethasone was prepared freshly by diluting 4× stock in DMSO with sterile PBS. 18 hours after injection, mice were weighed and euthanized, and thymi were harvested and both lobes were weighed. One lobe was immersed in OCT and snap-frozen for immunohistochemical staining to determine efferocytosis in situ, while the other lobe was mechanically disaggregated into single-cell suspension for flow cytometry (Wang et al., 2017). To evaluate in situ efferocytosis (Wang et al., 2017), frozen thymus specimens were cryosectioned at 4-μm and placed on Superfrost plus microscope slides. Sections were fixed in 4% PFA for 10 minutes and permeabilized in 1% Triton X-100 for 15 minutes. After rinsing with PBS for three times, sections were incubated with TUNEL staining reagents at 37° C. for 60 minutes and then washed three times with PBS. Sections were then blocked with 5% goat serum for 60 minutes at room temperature, followed by overnight incubation at 4° C. in anti-CD68 antibodies (Abcam) diluted in PBS supplemented with 5% BSA to label macrophage area. After washing in PBS, sections were incubated with fluorescently labeled secondary antibodies and counterstained with DAPI. Images were captured using ImageXpress Micro4 with a Nikon Plan Apo 40×/0.95 objective lens. For quantification, the TUNEL⁺ nuclei in close proximity or in contact with CD68+macrophages were counted as macrophage associated ACs, indicative of efferocytosis. The TUNEL⁺ nuclei without neighboring macrophages were counted as free ACs. The ratio of macrophage associated ACs to free ACs was calculated to represent the capability of efferocytosis by thymus macrophages.

To evaluate the percentage of Annexin V⁺ thymocytes by flow cytometry, mechanically disaggregated thymus cells were rinsed twice with cold DPBS containing 2% HI-FBS and 1 mM EDTA. Cells were then stained with AF647-conjugated Annexin V in Annexin V binding buffer (Invitrogen) at a concentration of 5×10⁶ cells/mL for 15 minutes at room temperature, followed by flow cytometry analysis.

### In Vivo Peritoneal Macrophage Efferocytosis Assay

Cre⁺ and Cre⁻ mice of 12 weeks old were injected intraperitoneally with 1×10⁷ TAMRA-stained apoptotic mouse thymocytes in 300 μl PBS. 15 minutes after injection, mice were euthanized, and peritoneal exudates were collected. The pelleted cells were blocked with CD16/32 (BioLegend) and then stained by FITC-conjugated F4/80 antibody (BioLegend) to label macrophages. The percentage of TAMRA+PMs was determined by flow cytometry (Moon et al., 2020).

siRNA-Mediated Gene Silencing and Transfection

Non-targeting siRNA and WDFY3-targeting siRNA (Dharmacon) were transfected using Lipofectamine RNAiMAX (Invitrogen) as per the manufacturer's recommendation. Briefly, human PBMCs were seeded at 4×10⁵ per well of 24-well culture dish for differentiation to HMDMs for 7 days with about 70% confluence. HMDMs were then transfected with a final concentration of 25 pmol siRNA and 1 μL Lipofectamine RNAiMAX in 500 μL Opti-MEM (Invitrogen) for 6 hours. A second transfection with the same condition was performed 18 hours after the completion of the first transfection. HMDMs were collected 48 hours from the start of the first transfection for assessing mRNA and protein expression, and efferocytosis capacities. Mouse complete blood cell count (CBC) and differential count Retro-orbital bleeding was performed to collect about 500 μL blood per mouse for complete blood count and differential count using a Heska Element HT5 by the diagnostic lab at the Institute of Comparative Medicine, Columbia University Irvine Medical Center.

### Statistical Analyses

Statistical analyses were performed using GraphPad Prism7. Data were tested for normality using the D'Agostino-Pearson test (when n>=8) or Shapiro-Wilk test (when n<8). Data that passed normality tests were analyzed using Student's t test for comparison of two groups. When n was less than or equal to 5 or when data did not pass normality test, the nonparametric Mann-Whitney test was used. Two-way ANOVA was performed for two independent variables (factors) with two or more groups. Tukey's post hoc test was applied to correct multiple comparisons. Data analyzed using parametric tests were presented as mean±standard error of mean (SEM), while data analyzed using nonparametric tests were presented as median±95% confidence interval (CI). Statistical significance of difference was accepted when P values were <0.05. The specific P values, the number of independent experiments or biological replicates (mice), and the number of technical replicates per independent experiment and biological replicate were specified in figures and figure legends.

TABLE

| | Phagocyte |
|---|---|
| | BMDMs |
| | Substrates |
| | Apoptotic Jurkat cells |
| Rank | (about 10 μm) |
| 1 | U2AF1 |
| 2 | ARPC4 |
| 3 | DYRK1A |
| 4 | WDR62 |
| 5 | POMT2 |
| 6 | PREX2 |
| 7 | HAVCR2 |
| 8 | BCL2L13 |
| 9 | TRAF3 |
| 10 | WDFY3 |
| 11 | VPS52 |
| 12 | SEC61A1 |
| 13 | KPNA6 |
| 14 | NCKAP1L |
| 15 | FCRL1 |
| 16 | VARS1 |
| 17 | CEBPB |
| 18 | TMEM200A |
| 19 | ACTR3 |
| 20 | SLC2A12 |
| 21 | TSC22D2 |
| 22 | ABHD17A |
| 23 | RAC1 |
| 24 | PREX1 |
| 25 | SH3GLB1 |
| 26 | SNX24 |
| 27 | CCDC115 |
| 28 | SETD1B |
| 29 | ABCE1 |
| 30 | CYFIP1 |
| 31 | PIAS1 |
| 32 | DCLRE1A |
| 33 | GIMAP4 |
| 34 | CEBPA |
| 35 | RTL8A |
| 36 | PRAMEF6 |
| 37 | TAS2R16 |
| 38 | ATP1B3 |
| 39 | ST8SIA2 |
| 40 | COMMD4 |
| 41 | SPTY2D1 |
| 42 | WASF2 |
| 43 | ABI1 |
| 44 | TREM2 |
| 45 | SHLD1 |
| 46 | GTF3C1 |
| 47 | TLE4 |
| 48 | ISY1 |
| 49 | OR10K1 |
| 50 | CDK9 |
| 51 | MGAT1 |
| 52 | VPS33A |
| 53 | ACTR2 |
| 54 | LACTB |
| 55 | FBXW12 |
| 56 | ELOVL5 |
| 57 | HNRNPK |
| 58 | SF3B2 |
| 59 | NFKBIA |
| 60 | FBXW2 |
| 61 | DENND3 |
| 62 | TMEM220 |
| 63 | CCDC9B |
| 64 | ALPK1 |
| 65 | UROD |
| 66 | ZBTB46 |
| 67 | ODAD2 |
| 68 | SOCS5 |
| 69 | PDCD1 |
| 70 | TAS1R1 |
| 71 | FIZ1 |
| 72 | GRIN2D |
| 73 | TOPAZ1 |
| 74 | ARPC3 |
| 75 | MTMR9 |
| 76 | TMEM98 |
| 77 | FBXO11 |
| 78 | CCDC88B |
| 79 | WNK1 |
| 80 | CEACAM4 |
| 81 | SERPINB3 |
| 82 | OPA3 |
| 83 | CTR9 |
| 84 | PRL |
| 85 | U2AF2 |
| 86 | METTL16 |
| 87 | POT1 |
| 88 | APOL4 |
| 89 | APOL1 |
| 90 | APOL3 |
| 91 | APOL2 |
| 92 | KATNBL1 |
| 93 | TDO2 |
| 94 | RHO |
| 95 | THAP3 |
| 96 | NPY4R |
| 97 | NPY4R2 |
| 98 | LYG2 |
| 99 | ATP6V0B |
| 100 | RPS23 |

### Example 2

Efferocytosis is the phagocytic clearance of dying cells by phagocytes, mainly macrophages. Efferocytosis is essential for maintaining homeostasis and resolving inflammation upon injury. Defective efferocytosis has been demonstrated in systemic lupus erythematosus (SLE or lupus), atherosclerosis, and cancer. Enhancing efferocytosis showed therapeutic promise in clinical studies, i.e., the anti-CD47 antibody targeting the “do not eat me” signal to enhance efferocytosis has demonstrated efficacy and safety in Phase 2 trials for cancers, and for reducing vascular inflammation, a key mechanism of atherosclerosis. Harnessing macrophage efferocytosis is therefore promising for diseases driven by defective efferocytosis.

Despite decades of research, it remains unclear if and how macrophages recruit different molecular machinery to engulf cargos that have distinct sizes and rigidity. Macrophages express receptors to recognize the ligands on the surface of cargo, e.g., phosphatidylserine on dying cells and their receptors such as MERTK and TIM4 on phagocytes. Yet little is known about the regulatory mechanisms beyond ligand-receptor recognition. A genome wide CRISPR screen using apoptotic T cells as the cargo was performed (Example 1). Intriguingly, it was discovered that WDFY3 was needed for the phagocytic clearance of dying cells (5-10 μm), but not zymosan (1 μm) or latex beads (4 or 10 μm). Consistently, WDFY3 was not identified in screens for the uptake of latex beads, zymosans, and myelin (500 nm). It was also confirmed that WDFY3 has a cytoplasmic localization and does not affect binding. Thus, WDFY3 is uniquely required for the uptake of dying cells. Mechanistic studies further revealed that WDFY3 is required for LC3 lipidation, the last step for phagosome-lysosome fusion and the subsequent lysosomal degradation of the engulfed cargo. Therefore, WDFY3 is essential in two major steps of efferocytosis, uptake and degradation; the latter is important for continual efferocytosis and resolution of inflammation.

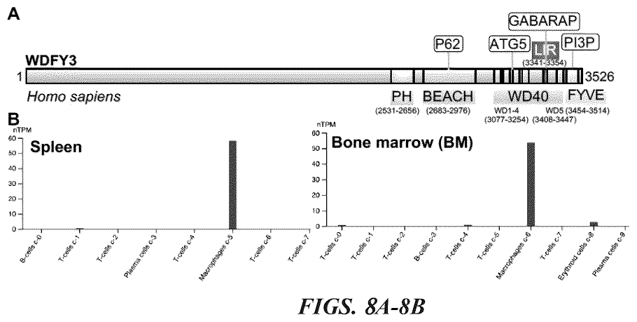

Discussed herein is how WDFY3 is differentially required for the uptake of dying cells, but not the less challenging cargos (rigid and non-deformable, or small); and how WDFY3 is required for the effective degradation of all engulfed cargos and the silence of inflammation. Further, as there is a lack of knowledge as to whether boosting macrophage efferocytosis results in the resolution of SLE, discussed herein is if enhancing WDFY3 is therapeutically effective in ameliorating lupus-like disorders (FIG. 8).

The molecular mechanisms of the differential requirement of WDFY3 in the uptake of dying cells. but not other cargos; and its nonselective requirement in degradation. Data show that Wdfy3 knockout impaired, while overexpression enhanced the uptake and degradation of dying cells. A proximity labeling experiment identified Arp2/3 complex and myosins being proximal (about 20 nm zone) to WDFY3. Using murine macrophages with Wdfy3 knockout or overexpression, and human macrophages with knockdown, the following were are tested: (A) WDFY3 is selectively required when macrophages are engulfing the more challenging cargos; (B) WDFY3 serves as a scaffold to bridge Arp2/3 and myosins during uptake of challenging cargos; (C) WDFY3 is required for the degradation of engulfed cargos non-selectively via regulating ROS production and promotes inflammation resolution, in addition to facilitating LC3 lipidation.

Impaired efferocytosis drives lupus-like disorders and if enhances efferocytosis protects against lupus-like disorders. Data show that acute injection of apoptotic T cells led to accumulation and delayed clearance in kidney, but not liver or spleen in myeloid-specific Wdfy3 knockout mice compared with controls, implicating kidney as being more vulnerable to apoptotic cell challenges. Chronic injection of apoptotic T cells led to lupus-like nephritis in myeloid-specific Wdfy3 knockout mice. Using the pristane-induced lupus model in myeloid knockout or overexpression mice, or with adoptive monocyte transfer, the following were tested: (A) myeloid-specific Wdfy3 knockout exacerbates, while (B) enhancing myeloid WDFY3 protects against lupus-like disorders. The following are assessed (1) kidney in situ efferocytosis index: determined by the ratio of macrophage-associated TUNEL+ apoptotic cells vs. TUNEL+ cells not bound to macrophages; (2) kidney pathology: glomeruli C1q and IgG deposition by immunohistochemical staining; (3) kidney macrophage inflammation: by RNA-seq; (4) kidney function: by urine albumin and serum creatinine; (5) systemic effects: serum autoantibodies and inflammatory cytokines by ELISA and Luminex assays, and splenomegaly.

Efferocytosis is the phagocytic clearance of dying cells by phagocytes, mainly macrophages (Boada-Romero et al., 2020). Effective efferocytosis is essential in maintaining homeostasis and resolving inflammation upon injury (Morioka et al., 2019). Defective efferocytosis has been demonstrated in autoimmune diseases, cancer, and atherosclerosis (Morioka et al., 2019; Doran et al., 2019; Mehrotra & Ravichandran, 2022). Enhancing efferocytosis showed therapeutic promises (Mehrotra & Ravichandran, 2022). Understanding more about efferocytosis will therefore have a broad impact on many diseases. Despite decades of research, there is a significant gap in knowledge as to how macrophages eliminate different cargos. In particular, beyond the known role of ligand-receptor binding for recognition, if and how macrophages recruit different molecular machinery to internalize and process corpses of different sizes and rigidity remain largely unknown. Understanding the specific principles of efferocytosis contributes to fundamental biological knowledge and facilitates specific targeting of dying cell clearance for therapeutic purposes.

Defective efferocytosis has been demonstrated in patients with systemic lupus erythematosus (SLE or lupus) (Herrmann et al., 1998; Baumann et al., 2002; Munoz et al., 2009; Huang et al., 2016; Geng et al., 2022), cancer (Gholamin et al., 2017; Advani et al., 2018), and atherosclerosis (Kojima et al., 2016; Jarr et al., 2021). Anti-CD47 antibody is a pro-efferocytic therapy that blocks the “do not eat me” signal to enhance macrophage efferocytosis. Phase 2 trials established the safety and efficacy of anti-CD47 in improving cancer prognosis (Gholamin et al., 2017; Advani et al., 2018), as well as reducing vascular inflammation (Jarr et al., 2021), a key mechanism of atherosclerosis. In light of the clinical success of pro-efferocytic therapy and the cellular and pathological data supporting impaired efferocytosis in lupus patients (Mehrotra & Ravichandran, 2022; Beningo & Wang, 2002; Jaumouille et al., 2019), exploring targeting efferocytosis in SLE and lupus nephritis may allow for new platforms of treatments.

Early studies recognized that macrophages can take up rigid cargos more effectively than soft ones (Beningo & Wang, 2002; Jaumouille et al., 2019), likely because the soft cargos are deformable thus requiring stronger mechanical force to be generated. Engulfment of large cargos also requires the involvement of different molecular machinery (Schlam et al., 2015). Specifically, the engulfment of larger beads (about 5 μm and rigid), but not the small ones, requires phosphoinositide 3-kinase (PI3K)-mediated PtdIns(3,4,5)₃ production and PtdIns(3,4,5)₃-dependent recruitment of GTPase-activating proteins (GAPs) that inactivates Rho GTPases RAC/CDC42, therefore allowing the cycling of F-actin assembly and disassembly for engulfment (Schlam et al., 2015). Apoptotic cells (ACs) are typically between 5-10 μm and are deformable. ACs expose phosphatidylserine (PtdSer) for recognition by receptors on phagocytes, such as MERTK and TIM4. It is unknown if the mechanical properties of dying cells require specific mechanisms for their internalization upon recognition.

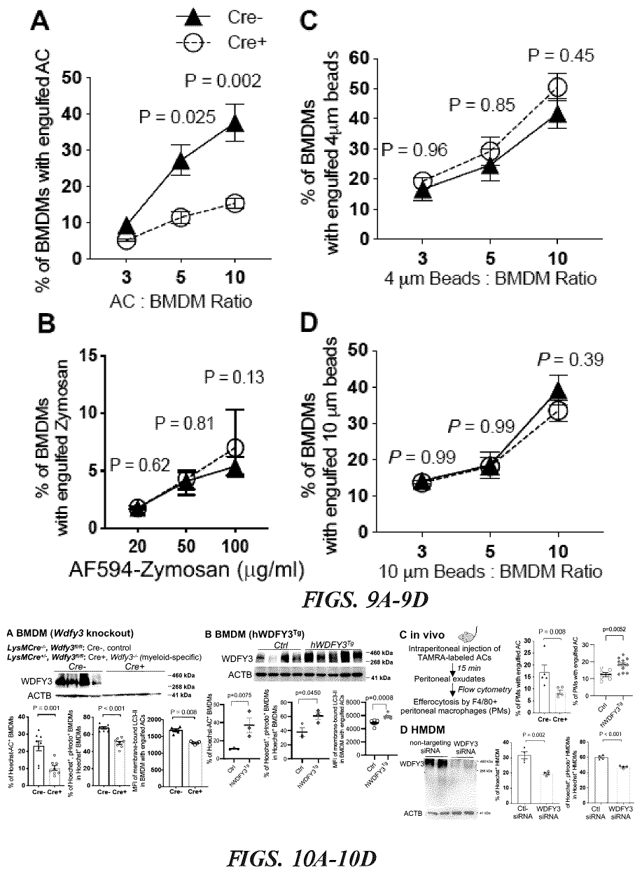

To identify efferocytosis-specific mechanisms, a genome-wide CRISPR screening was performed using apoptotic T cells as the cargo and discovered WDFY3 as being required for the uptake and degradation of the engulfed ACs (Shi et al., 2022a). WDFY3 is a large 400 kDa protein with several known functional domains (Shi et al., 2022a) and is most abundantly expressed in macrophages vs. other immune cells (FIG. 9). Intriguingly, it was confirmed that WDFY3 is dispensable for the uptake of rigid beads and smaller cargos like zymosans (FIG. 10). Consistent with the results, previous genome-wide screening using various cargos but not dying cells did not identify WDFY3 (Hedgecock et al., 1983; Ramet et al., 2002; Silva et al., 2007; Santulli-Marotto et al., Haney et al., 2018; Kamber et al., 2021), illuminating the specific principles of efferocytosis beyond the universal mechanisms of phagocytosis.

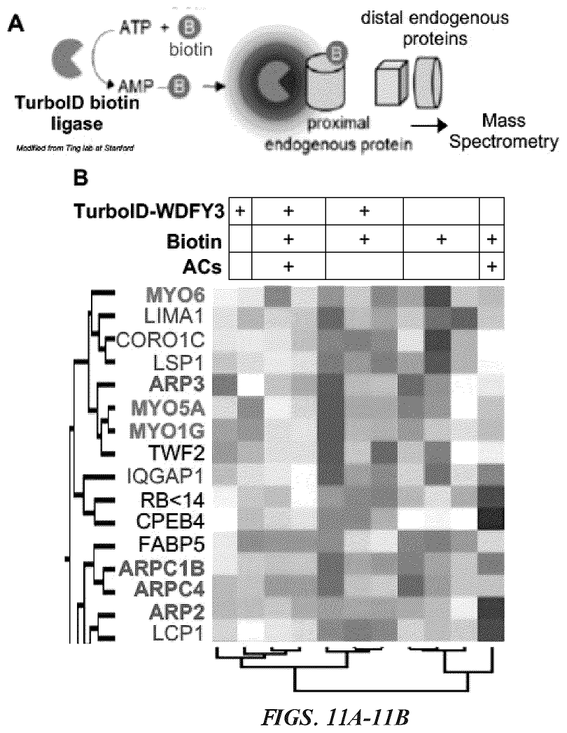

Data support that Wdfy3 knockout impaired, while overexpression enhanced uptake and lysosomal acidification of dying cells in murine bone-marrow derived macrophages (BMDM) (FIG. 11A-B). Uptake of ACs by Wdfy3 knockout BMDMs requires longer time from binding to complete internalization (Shi et al., 2022). Wdfy3 knockout, but not control BMDMs, showed an accumulation of F-actin surrounding the newly internalized ACs (Shi et al., 2022), supporting defects in efficient F-actin disassembly. WDFY3 directly interacts with GABARAP to facilitate LC3 lipidation and the subsequent lysosomal degradation of engulfed dying cells (Shi et al., 2022), consistent with the previously known role of WDFY3 in bridging autophagic machinery to facilitate lysosomal degradation of aggregated proteins in neuronal cells (Filimonenko et al., 2010).

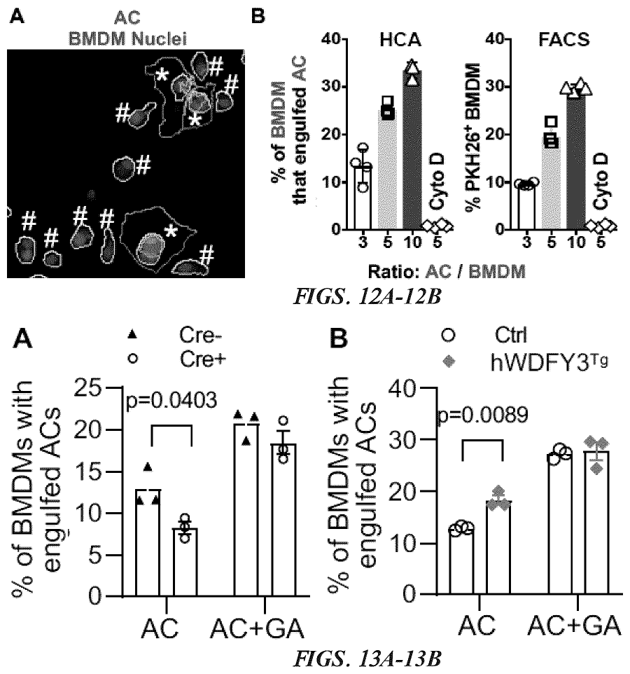

The central questions to be addressed were: (1) why and how is WDFY3 required for the uptake of dying cells, but not other cargos; (2) are WDFY3 required for the degradation of different cargos and are there molecular mechanisms by WDFY3 in regulating degradation beyond its known role in facilitating LC3 lipidation. To leverage unbiased methods for hypothesis generation, proximity labeling was performed to identify proteins in close proximity to WDFY3 (about 20 nm zone by using the TurboID biotin ligase as illustrated in FIG. 12A (Cho et al., 2020; Qin et al., 2021)). A number of subunits of Arp2/3 complex, myosins, and proteins involved in the regulation of F-actin dynamics (FIG. 12B) were identified. RNA-seq was used to identify the pathways altered by Wdfy3 knockout and validated that the impaired AC engulfment-induced reactive oxygen species (ROS) production may represent the additional mechanisms by which Wdfy3 knockout causes impaired degradation (FIG. 10).

In homeostasis, the clearance of apoptotic material is rapid, efficient, and anti-inflammatory, preceding the release of nuclear constituents from the dying cells (Trzeciak et al., 2021; Yin & Heit, 2021). In patients with lupus, there is increased cell death and impaired clearance of dying cells by phagocytes (Herrmann et al., 1998; Baumann et al., 2002; Munoz et al., 2009; Huang et al., 2016; Geng et al., 2022). Uncleared dying cells become secondarily necrotic (Gaipl et al., 2007). Impaired degradation of nuclear material derived from necrotic cells contribute to the exacerbation of inflammation and autoimmunity (Munoz et al., 2009). Experimental studies support that impaired dying cell clearance itself is sufficient to induce lupus-like disorders and exacerbate autoimmunity (Martinez et al., 2016). However, there is a gap in knowledge as to whether boosting of lupus and lupus nephritis. The role of WDFY3 in efferocytosis was confirmed in vivo in mice and in human macrophages (FIG. 11C-D). Chronic apoptotic thymocyte injection to myeloid-specific Wdfy3 knockout mice led to lupus-like disorders and lupus nephritis, including increased autoantibodies to ANA and kidney deposition of IgG and C1q (FIG. 9), while WDFY3 overexpression ameliorates the development of autoantibodies (FIG. 8). Leveraging the data, it is determined if WDFY3 is protective in lupus using pristane-induced lupus mouse models, therefore further establishing the premise of targeting efferocytosis in lupus and lupus nephritis.

The differential requirement of WDFY3 in the uptake of the more challenging cargos. This study provides an example of how macrophages involve differential molecular machinery when dealing with the more challenging cargo. Proximity labeling data imply that WDFY3 likely serves as a scaffold protein for Arp2/3 complex and myosins in the regulation of F-actin dynamics. Live-cell imaging and pull-down was used to visualize and validate the interaction during the uptake of an array of cargos, i.e., large and deformable ACs, large and rigid cells, and small cell debris. The results will contribute to the fundamental knowledge of how cytoskeleton dynamics are regulated by WDFY3 to deal with the clearance of the more challenging cargos, which is dispensable for the uptake of the less challenging cargos.

The Yamamoto lab first identified that WDFY3 recruits autophagic complex for clearance of intracellular aggregated proteins in neurons (Filimonenko et al., 2010), thus playing important roles in Huntington's disease (Fox et al., 2020). It was discovered that WDFY3 is also essential for the uptake and degradation of engulfed dying cells by macrophage (Shi et al., 2022). WDFY3 overexpression led to increased clearance of aggregated proteins in neurons (Yamamoto lab) and increased uptake and degradation of dying cells by macrophages via enhancing LC3 lipidation (FIG. 11B). The role of WDFY3 in cargo engulfment-induced ROS production, which is essential for downstream phagosome-lysosome fusion and acidification for degradation (Shekhova, 2020; Martinez et al., 2015), is further validated. It is speculated that WDFY3 is nonselectively required for the degradation of all engulfed cargos because the transcriptomic changes in genes regulating phagocyte ROS production should lead to phenotypic changes regardless of the type of cargos.

WDFY3 may represent a potential therapeutic target for pro-effeocytotic therapy in lupus. The ongoing work continues to address the role of macrophage WDFY3 in atherosclerosis using atherosclerosis-prone mouse models and human functional genomics, including PheWAS for cardiometabolic traits and human iPS-derived macrophages to model WDFY3 variants. Wdfy3 deficiency exacerbates, while WDFY3 overexpression ameliorates lupus-like disorders induced by chronic injection of apoptotic thymocytes. In light of the promise of pro-efferocytic therapy in cancers and atherosclerosis (Gholamin et al., 2017; Advani et al., 2018; Jarr et al., 2021), and the clinical premise that efferocytic clearance of dying cells and debris is impaired in lupus patients (Herrmann et al., 1998; Baumann et al., 2002; Huang et al., 2016; Geng et al., 2022), it is established that enhancing efferocytosis may be a potential therapeutic approach for lupus and lupus nephritis.

### Determine the Molecular Mechanisms of the Differential Requirement of WDFY3 in the Uptake of Dying Cells, but not Other Cargos, and its Nonselective Requirement in Degradation.

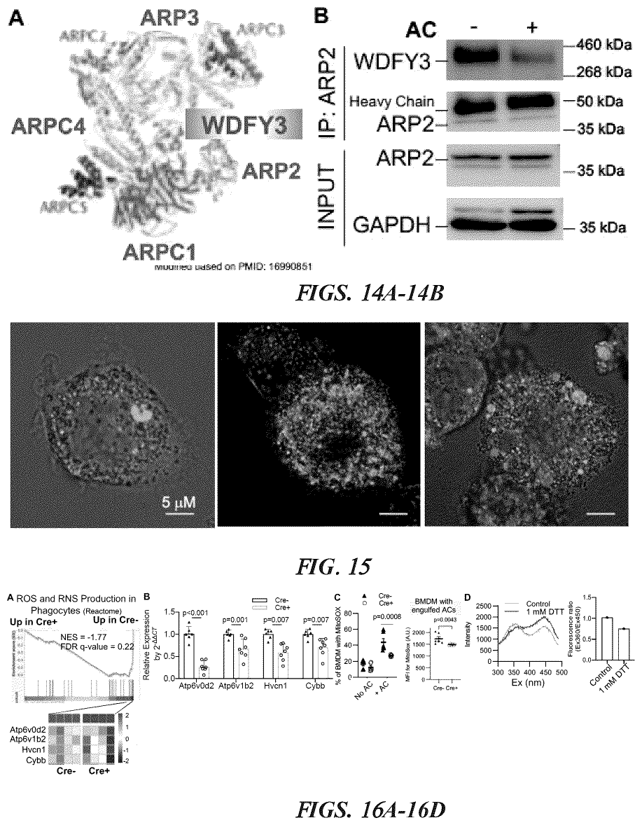

How macrophages engage different molecular machinery to deal with different cargos is minimally understood. WDFY3 was discovered as being required for efferocytosis of dying cells by macrophages (Shi et al., 2022). WDFY3 deficiency does not affect the engulfment of the rigid beads (with sizes similar to that of ACs) and small zymosan particles (FIG. 10) (Shi et al., 2022), supporting the differential requirement of WDFY3 in engulfing dying cells. What is special about dying cell clearance by macrophages? And why uptake of beads or small zymosan particles do not require WDFY3? Previous studies observed that macrophages engulf rigid cargos more effectively than soft cargos (Beningo & Wang, 2022; Jaumouille et al., 2019), e.g., increasing cell rigidity by treating murine red blood cells (RBC) with glutaraldehyde (GA) leads to more effective clearance (Sosale et al., 2015). Since the beads tested are of similar size to dying cells but are rigid, it was hypothesized that being soft and deformable, instead of rigid, is the key characteristics of cargos to require WDFY3 for clearance. Indeed, data using GA-treated ACs led to more effective engulfment in both control and Wdfy3 knockout BMDMs and abolished the difference between the two groups (FIG. 14A). Consistently, GA-treated AC led to increased uptake by both control and hWDFY3^{Tg} BMDMs (FIG. 14B).

AC and BMDM binding were not affected by Wdfy3 knockout (Shi et al., 2022). Wdfy3 knockout led to an increase in the amount of time needed from binding to complete internalization (Shi et al., 2022). In Wdfy3 knockout BMDM, it was observed that F-actin (labeled by siR-Actin) forms ring-like structure surrounding the engulfed cargos, while in control BMDMs, the ring-like structure is rarely seen once ACs are engulfed (Shi et al., 2022). The ring-like structure is also rarely seen in BMDMs engulfing beads (Shi et al., 2022). Thus, WDFY3 deficiency leads to dysregulated F-actin dynamics, which appears essential in AC uptake. Previous studies indeed support that the engulfment of large cargos requires the synchronized F-actin assembly and disassembly (Schlam et al., 2015). Therefore, it was hypothesized that size and rigidity affect how macrophages engage different molecular machinery to deal with the cargo and WDFY3 plays an important role in the process.

A recent study already confirmed that WDFY3 is required for LC3 lipidation through direct interaction with GABARAP (Shi et al., 2022). This is aligned with prior data supporting that WDFY3 is required for the macroautophagic clearance of intracellular protein aggregates (Filimonenko et al., 20210; Fox et al., 2020). The remaining question is if the role of WDFY3 in regulating the degradation of the engulfed cargo entirely relies on regulating LC3 lipidation? This is unlikely the case because when lentiviral vectors are used to express C-terminal WDFY3 in Wdfy3 knockout BMDM, the reconstitution of C-WDFY3 completely rescued impaired LC3 lipidation, but lysosomal acidification was only partly restored (Shi et al., 2022). C-WDFY3 was also not sufficient to restore uptake (Shi et al., 2022). Therefore, it was hypothesized that WDFY3 mediates degradation through additional mechanisms. RNA-seq data of Wdfy3 knockout vs. control BMDMs identified only a small number of differentially expressed genes, i.e., 31 downregulated and 23 upregulated genes in Wdfy3 knockout vs. controls (Shi et al., 2022). Yet pathway analysis supports an enrichment of downregulated genes in the pathway term “ROS and RNS production in phagocytes” (FIG. 17A). qPCR validation supports a modest downregulation of the leading-edge genes contributing to the pathway enrichment (FIG. 17B). The hypothesis that ROS production or cargo engulfment-induced ROS upregulation may be impaired in Wdfy3 knockout BMDMs is explored. ROS production is required for cargo degradation (Shekova, 2020; Martinez et al., 2015; Xia et al., 2019), which may represent the additional mechanisms of WDFY3 in the degradation of engulfed cargos. Effective degradation is also essential for the resolution of inflammation (Perry et al., 2019).

Thus, WDFY3 is differentially required for the uptake of dying cells, but not the less challenging cargos; WDFY3 is, however, required for the effective degradation of all engulfed cargos and the silence of inflammation.

### Experimental Models:

Mice models: All mice are on C57BL/6J background. Both male and female mice at about 2 months old are used.

Myeloid-specific knockout: Wdfy3_{fl/fl} mice originally obtained from Yamamoto lab are now maintained in the Zhang lab (Shi et al., 2022). Myeloid-specific Wdfy3 knockout mice are maintained by crossing LysMCre (JAX #004781) with Wdfy3^{fl/fl} mice. LysMCre^{+/-}Wdfy3^{fl/fl} mice (Cre+) have myeloid-specific knockout of Wdfy3, while the LysMCre^{-/-}Wdfy3^{fl/fl} mice (Cre⁻) with be the littermate controls (FIG. 11A shows successful knockout). Myeloid-specific overexpression: For data, the whole body hWDFY3^{Tg} mice were generated by crossing floxed-human WDFY3 transgenic mice with Hprt^{Cre} by Yamamoto lab. The colonies are maintained in the Zhang lab by het×het breeding to obtain all the genotypes. Wild-type mice in the colonies are used as controls (FIG. 11B shows successful overexpression). For in vitro studies using BMDMs, the whole-body overexpression is sufficient. For myeloid-specific overexpression, the hWDFY3^{Tg fl/fl} mice will be bred with LysMCre mice. LysMCre^{+/-}hWDFY3^{Tg fl/fl} have myeloid-specific overexpression of WDFY3, LysMCre^{-/-}hWDFY3^{Tg fl/fl} will be the littermate controls. LysMCre was chosen because of high efficiency of gene editing in myeloid cells (Shi et al., 2018; Abram et al., 2014).

Murine macrophages: Bone marrow (BM) cells are isolated by flushing femurs and tibia and differentiated for 7-10 days (Shi et al., 2022). BMDMs are differentiated in DMEM with 10% FBS, 20 ng/mL mM-CSF, and 2 mM L-Glutamine (Shi et al., 2022).

Human macrophages: Monocyte-derived macrophage (HMDM) (Zhang et al., 2015) with knockdown by siRNA (Shi et al., 2022) that shows >80% knockdown efficiency is used and as well as nontargeting siRNA (Dharmacon) as controls (FIG. 11D shows successful knockdown).

AC preparation and labeling for efferocytosis assays: Jurkat cells (about 10 μm, a human T cell line) and mouse thymocytes (about 5 μm, isolated from mouse thymi) are treated with staurosporine for 3 hours to induce apoptosis. The method routinely yields greater than 90% Annexin V+ ACs (Shi et al., 2022). GA treatment at 10 mM for 1-minute leads to an increase in cell rigidity (Sosale et al., 2015). Mouse RBC are isolated from whole blood freshly and opsonized by IgG (Sosale et al., 2015). Storage at 4° C. for one-week or treatment by GA increases rigidity (Sosale et al., 2015). Mouse RBC are freshly prepared because commercial RBCs are often stored in cold conditions for shipment and thus likely already have increased rigidity. Cells will be labeled by TAMRA (10 μg/mL, 25 minutes, Invitrogen #C1171) that labels both DNA and protein (Shi et al., 2022; Bi et al., 2022). To assess uptake and acidification, ACs are fluorescently labeled by TAMRA (pH-insensitive) and pHrodo (20 ng/mL, 30 minutes, ThermoFisher #P35373, pH-sensitive and only fluoresces in acidic environments) (Shi et al., 2022; Bi et al., 2022). RBC are labeled by PKH26 which labels membrane lipids (Sigma PKH26GL-1KT) (Shi et al., 2022). Cell debris is prepared by passing labeled ACs 10 times through 27G needles (Puigdellivol et al., 2021). Labeled cargos are incubated with BMDMs or HMDMs at a ratio of 5:1 for cargo: macrophages (Puigdellivol et al., 2021; Wang et al., 2017), or at 30 mg of debris (equivalent to about 1×10\hat{ }5 dead cells) per well of 24-well plate, for one hour for efferocytosis. The % of macrophages that engulfed cargo will be quantified by FACS (Shi et al., 2022).

### Experimental Design and Methodology:

WDFY3 is required when macrophages are engulfing the more challenging (soft and large) cargos. It is hypothesized that Wdfy3 knockout in BMDM or WDFY3 knockdown in HMDM only impairs the uptake of ACs, but not rigid or small cargos; it is also hypothesized that hWDFY3^{Tg} increases the uptake of AC but will not further enhance the uptake of other rigid or small cargos, because the engulfment of the “less challenging” cargos do not require WDFY3-mediated mechanisms.

An array of particles, including AC (large and deformable), GA-treated AC (large and rigid, because GA functions as a crosslinker and fixative to increase cell rigidity), and AC debris (small) are used. Freshly isolated RBC, RBC stored at 4° C. for one week, and GA-treated RBC are also tested. Similarly, it is hypothesized that stored and GA-treated RBC engulfment does not require WDFY3.

WDFY3 serves as a scaffold to bridge F-actin and myosins during the uptake of challenging cargos. It is hypothesized that WDFY3 serves as a scaffold protein for the Arp2/3 complex, myosin V and VI, and other proximal proteins identified by proximity labeling, e.g., IQGAP1 (Briggs & Sacks; 2003; Mateer et al., 2004), LSP1 (Jongstra-Bilen et al., 1992), LIMA1 (Maul et al., 2003; Taha et al., 2019), LCP1 (Morley, 2012), CORO1C (Cai et al., 2008), and TWF2 (Hakala et al., 2021) that had known roles in F-actin binding or dynamics. It is speculated that during uptake, the activation of RAC/CDC42 is relayed to Arp2/3 complex via the Wiskott-Aldrich syndrome proteins (WASPs) (Pollard & Borisy, 2003)—the C terminus of N-WASP binds to the Arp2/3 complex (Rohatgi et al., 1999), thus recruiting Arp2/3 complex together with WDFY3 and other proximal proteins to the phagocytic cup. Myosin V and VI are responsible for filopodia extension (Masters & Buss, 2017; Wang et al., 1996). All the WDFY3 proximal proteins together facilitate the synchronized F-actin assembly and disassembly and filopodia extension while taking up large and deformable cargos. It was hypothesized that without WDFY3, the recruitment of myosins and other proximal proteins to the phagocytic cup is less efficient. It is hypothesized that during engulfment of the rigid or small particles, WDFY3 is also recruited to the phagocytic cup together with Arp2/3 complex and other proximal proteins, but F-actin accumulation and cargo internalization are not affected.

Are there direct protein-protein interactions between WDFY3 and the proximal proteins identified in proximity labeling? Pull-down of WDFY3 is performed and the proximal proteins blotted or pull down of the proximal proteins is performed and WDFY3 blotted. Lentiviral transduction of C-WDFY3 (with 3-HA tag) (Shi et al., 2022) or hWDFY^{Tg} (with flag tag) is employed for pull-down because pull-down by WDFY3 antibodies is technically challenging and not feasible likely due to the large size of WDFY3 (400 kDa). Pull-down in BMDM with or without AC incubation is performed in order to determine if the quantitative difference shown in proximity labeling experiments can be replicated. (FIG. 15 shows the direct protein-protein interaction between ARP2 and WDFY3, supporting the hypothesis and feasibility.)

Are there colocalization between WDFY3 and its proximal proteins, and F-actin network during engulfment? A THP-1 human monocytic cell line was developed with endogenously tagged WDFY3 with eGFP at the N-terminal by CRISPR-mediated knockin for live-cell imaging to visualize endogenous WDFY3 (FIG. 16 supports successful tagging and the cytoplasmic localization of WDFY3). The colocalization of WDFY3 and Factin (labeled by siR-actin, a fluorogenic, cell-permeable probe based on an F-actin binding natural product jasplakinolide), and other proximal proteins is performed by IHC staining with or without AC engulfment. Macrophages are transfected with LifeAct-tagGFP2 mRNA encoding a fusion protein of LifeAct and a GFP reporter (Xu & Du, 2021) for visualization of F-actin and perform live-cell imaging in order to visualize the colocalization and dynamics of WDFY3 and F-actin network. It is determined if myosin V and VI and other proximal proteins show reduced recruitment to the phagocytic cup with Wdfy3 knockout or knockdown, or increased recruitment in hWDFY3^{Tg} BMDMs.

In addition or Arp2/3 complex and myosin V and VI, IQGAP1 (Briggs & Sacks, 2003; Mateer et al., 2004), LSP1 (Jongstra-Bilen et al., 1992), LIMA1 (Maul et al., 2003; Taha et al., 2019), LCP1 (Morley, 2012), CORO1C (Cai et al., 2008), and TWF2 (Hakala et al., 2021) have been reported in the literature to bind with F-actin or Arp2/3 complex. Similarly, it is hypothesized that their close proximity/interaction with WDFY3 allows efficient recruitment to the phagocytic cup to synergistically regulate F-actin dynamics for AC engulfment.

What is the role of the N-terminal WDFY3? The data of proximity labeling was performed with CWDFY3₍₂₅₄₃₋₃₅₂₆₎, the C-terminal WDFY3 has all known functional domains (FIG. 9A). Because of the large size, full-length WDFY3 cannot be packaged for lentiviral delivery in order to perform proximity labeling. Proximity labeling is separately performed with the N-terminal WDFY3 for WDFY3₍₁₋₁₂₅₀₎ and WDFY3₍₁₂₅₁₋₂₅₄₂₎, respectively. It is hypothesized that the N-WDFY3 may or may not show the same binding partner, but N-terminal and full-length WDFY3 are essential for the maximal efficiency in the recruitment of the proximal proteins identified by proximity labeling using the CWDFY3.

How does hWDFY3^{Tg} increases uptake? It is hypothesized that hWDFY3^{Tg} increases the recruitment efficiency of proximal proteins and will increase the percentage of macrophage that can engulf, and also shorten the time required from binding to phagocytic closure as determined by live-cell imaging.

How is WDFY3 involved while engulfing rigid or small particles that do not require WDFY3 for engulfment? It is hypothesized that during engulfed of the rigid or small particles, WDFY3 is also recruited to the phagocytic cup together with Arp2/3 complex and other partners. Without WDFY3, the recruitment of proximal proteins is reduced. But either knockout or overexpression will not affect internalization time nor F-actin accumulation, because the molecular machinery without WDFY3 is sufficient for the engulfment of the rigid or small cargos.

WDFY3 is required for the degradation of engulfed cargos non-selectively via regulating ROS production and promotes inflammation resolution.

Is WDFY3 required for the degradation of all engulfed cargo? It is hypothesized that once cargos are engulfed, WDFY3 is required for the LC3 lipidation, lysosomal acidification, and cargo degradation, i.e., WDFY3 is nonselectively required for the degradation of all cargos tested. LC3 lipidation, lysosomal acidification, and degradation of cargos is determined, including ACs, GA-treated ACs, AC debris, and RBC and GA-treated RBC.

Uptake and acidification (Shi et al., 2022): Upon labeling with both TAMRA (red) and the pH-sensitive pHrodo-green, cargos are incubated with BMDMs or HMDMs at a ratio of 5:1 or at 30 mg of debris (equivalent to about 1×10\hat{ }5 dead cells) per well of 24-well plate to allow efferocytosis. For FACS-based assay, macrophages are collected at 1 hour. The % of TAMRA+ macrophages represents uptake, i.e., the macrophages engulfed cells or debris. The % of TAMRA+pHrodo+ macrophages in TAMRA+ macrophages represents acidification, i.e., the % of macrophages with engulfed and acidified cargos. For imaging, cells are imaged every 15 minutes using ImageXpress Micro4 HCA imager for 2 hours to quantify the pHrodo intensity over time that represents acidification efficiency.

LC3 lipidation (Shi et al., 2022; Martinez, 2020): macrophages are incubated with TAMRA-labeled cargos for 1-hour efferocytosis, then collected and resuspended in 300 μL cold PBS with 20 μg/mL digitonin and incubated on ice for 10 minutes to permeabilize cells and allow non-membrane bound LC3 to be removed from cells. Permeabilized macrophages are then incubated with anti-LC3A/B-FITC antibody to stain the membrane-bound lipidated LC3-II within the cells. After staining, macrophages are collected, and LC3-II signal will be quantified by FACS.

Degradation (Shi et al., 2022: Perry et al., 2019): For FACS-based assay, cargos are labeled with TAMRA and incubated with macrophages. Upon 1 hour, unengulfed cargo are washed away and macrophages are collected for baseline fluorescence intensity of the engulfed cargo. Macrophages are cultured for 18 hours to allow degradation and the percentage of decrease in TAMRA signal is calculated as the degradation rate. For imaging, macrophages are imaged every hour for 18 hours; the TAMRA intensity is quantified to plot the degradation rate over time.

Is WDFY3 required for cargo engulfment-induced ROS production? RNA-seq analysis suggests that Wdfy3 knockout led to downregulation of genes enriched in the “ROS and RNS production in phagocytes” pathway (FIG. 17A). There are a number of genes in the pathway showing modest fold change with reduced expression in Wdfy3 knockout BMDMs (FIG. 17B). It is hypothesized that modest changes in the genes belonging to the same pathway may lead to functional impact. It was confirmed that baseline mitochondrial ROS production was not altered. AC engulfment enhances mitochondrial ROS production and AC engulfment-induced ROS production is lower in Wdfy3 knockout BMDM (FIG. 17C). It is determined if Wdfy3 knockout reduces, while hWDFY3^{Tg} further enhances the efficiency of ROS production upon engulfment of different cargos. Three methods are used: (1) MitoSOX to determine mitochondria-derived superoxide and quantify by FACS; (2) cytoroGFP-iE (Birk et al., 2013) transfection to determine the redox status (roGFP is a fluorescent protein chimera that changes its excitation maximum from 488 nm to 405 nm based on a highly efficient redox relay. Using ratiometric detection of the emission at 515 nm by a microplate reader, a reduced Ex360/Ex450 indicates reductive stress (as supported by DTT treated BMDMs) while an increased Ex360/Ex450 ratio indicates oxidative status (FIG. 10D supports feasibility); (3) CellROX to determine cellular and phagosomal ROS by imaging analyses.

It is also determined if mitochondria homeostasis is affected by Wdfy3 knockout because previous studies suggested impaired mitophagy in neuronal cells with Wdfy3 knockout (Napoli et al., 2018). Mitochondrial mass (MitoTracker) and mitochondrial membrane potential (TMRM, Invitrogen T668) will be determined by FACS (Chen et al., 2019). MitoTracker imaging will also be used to assess mitochondria morphology for (1) mitochondrial content (cell area occupied by mitochondria, average area per mitochondrion using Mito-Morphology Macro in ImageJ (Dagda et al., 2009); (2) mitochondrial morphology (circularity index with a more tubular shape being closer to a value of 1); (3) network integrity (number of network per cell and mean branch length using MiNA macro in ImageJ) (Valente et al., 2017). Smaller sizes, less tubular in shape, and fewer networks with smaller branches indicate defective mitochondria.

Is WDFY3 required for silencing inflammation upon cargo engulfment? It is hypothesized that effective degradation of the cargo is essential for silencing inflammatory response. It is determined if Wdfy3 knockout impairs, while hWDFY3^{Tg} ameliorates inflammatory responses upon engulfment using RNA-seq. To minimize informatics challenges, RBCs are used as the cargo because RBC do not have nuclei therefore the observed transcriptomic responses are attributable to the BMDMs (Yurdagul et al., 2021). RBC-incubated BMDMs are collected at 3 hours and 18 hours, isolate RNA, and perform RNA-seq at 20M 100-bp paired-end reads.

### Results

Wdfy3 knockout or hWDFY3 overexpression only likely affects the engulfment of the more challenging cargos, e.g., ACs and IgG-opsonized freshly isolated RBC, but not ACs or RBCs with increased rigidity. The uptake of debris of small size is also not affected.

WDFY3 may serve as a scaffold protein to bridge the Arp2/3 complex and myosins and other proximal proteins responsible for F-actin dynamics. During uptake, WDFY3 and the proximal proteins are recruited to the phagocytic cup. WDFY3 ensures effective recruitment and synchronized F-actin assembly and disassembly which are essential for the engulfment of the more challenging cargos. At baseline, WDFY3 are likely colocalized with those protein partners, while during uptake, WDFY3 remains in interaction with Arp2/3 complex, while other protein partners recruited with WDFY3 participate in filopodia extension and phagocytic cup closure and may not be colocalized with WDFY3 (as implied by proximity labeling data showing lower proximity with AC treatment). WDFY3 is also likely recruited with Arp2/3 together during the engulfment of the rigid cargos that also involve RAC/CDC42/N-WASP activation, but the lack of WDFY3 does not affect cargo engulfment. It is expected that WDFY3 overexpression would increase both the percentage of macrophages that engulf the challenging cargos and shorten the time required from binding to complete internalization.

WDFY3 is likely differentially required for the uptake of the more challenging cargos. WDFY3 contributes to the degradation of the engulfed cargos through enhancing mitochondrial ROS production and LC3 lipidation, thus the degradation of the engulfed cargos is nonselective (for the cargos being tested). Acidification and degradation of GA-treated ACs and AC debris, or GA-treated RBCs, though not impaired for uptake, show impaired lysosomal acidification and degradation with Wdfy3 knockout, while hWDFY3^{Tg} enhances their degradation. Cargo-induced ROS production is impaired in Wdfy3 knockout and enhanced in hWDFY3^{Tg}, while baseline ROS or mitochondria function are comparable between control and Wdfy3 knockout BMDM (because baseline ROS and LC3 lipidation are not altered by Wdfy3 knockout (Shi et al., 2022a).

Baseline transcriptome signature is similar among all groups. Wdfy3 knockout leads to enhanced inflammatory responses due to impaired degradation. hWDFY3S leads to silenced inflammatory response due to more effective degradation.

### Alternative Strategies:

Using beads with different rigidity and size can provide a controlled and precise assessment of the effects of size and rigidity, yet the surface labeling may not resemble that of dying cells and debris clearance. Beads can be incorporated to further dissect if specific ligand-receptor recognition, e.g., Fc-gamma receptors, phosphatidylserine receptors, or complement receptors, may be differentially involved for uptake and/or cargo acidification.

The overexpression effects in BMDM with human WDFY3 transgenic overexpression is tested. CRISPRa activation in human iPS-derived macrophages can be used to further confirm the human relevance of WDFY3 overexpression which the PI has expertise (Shi et al., 2019; Zhang et al., 2017; Zhang et al., 2015; Zhang et al., 2019). Lipid nanoparticle-mediated mRNA delivery can also be used.

N-terminal proximity labeling reveals if the N-terminal WDFY3 has similar or different proximal proteins.

In Hela cells, WDFY3 localizes with early endosomes and cellular protrusions (Soreng et al., 2022). Hela cells with WDFY3 knockout migrate faster but had impaired directional cell migration (Soreng et al., 2022). The data did not show differences in migration, possibly suggesting a cell type-specific role of WDFY3. The localization of WDFY3 with endosome can be explored. In non-muscle cells, actin filaments form an internal track system for vesicle and organelle transport that is powered by motor proteins such as myosin V and myosin VI (Carvalho et al., 2013). These myosins transport cargo at rates much faster than diffusion (Carvalho et al., 2013). One alternative hypothesis is that WDFY3 deficiency also affects vesicle transport.

### Determine how Impaired Efferocytosis Drives Lupus-Like Disorders and if Enhancing Efferocytosis Protects Against Lupus-Like Disorders.

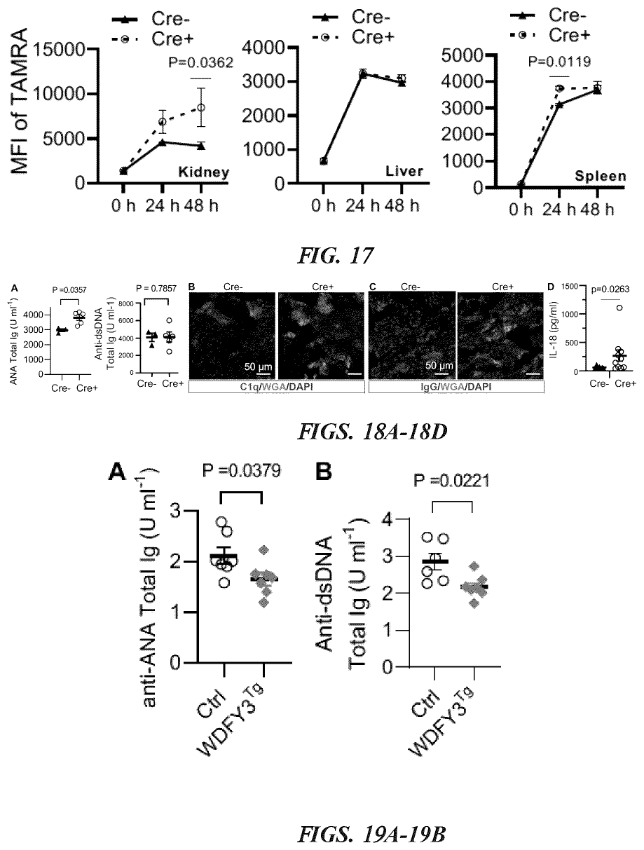

Kidney damage is one of the most common health problems caused by lupus (Liu et al., 2013). In adults who have lupus, as many as 5 out of 10 will have kidney disease (Liu et al., 2013). Although women are more likely to develop lupus, men develop lupus nephritis more than women (Hahn et al., 2012). Lupus nephritis can lead to worsened kidney function or kidney failure (Lech & Anders, 2013). Knockout of Wdfy3 led to the accumulation of ACs and delayed clearance in the kidney, but not liver or spleen upon one time AC injection (i.v. for 1×10\hat{ }7 apoptotic thymocytes) (FIG. 18), implicating that kidney may be more vulnerable to AC challenges, either due to higher burden or poorer clearance.

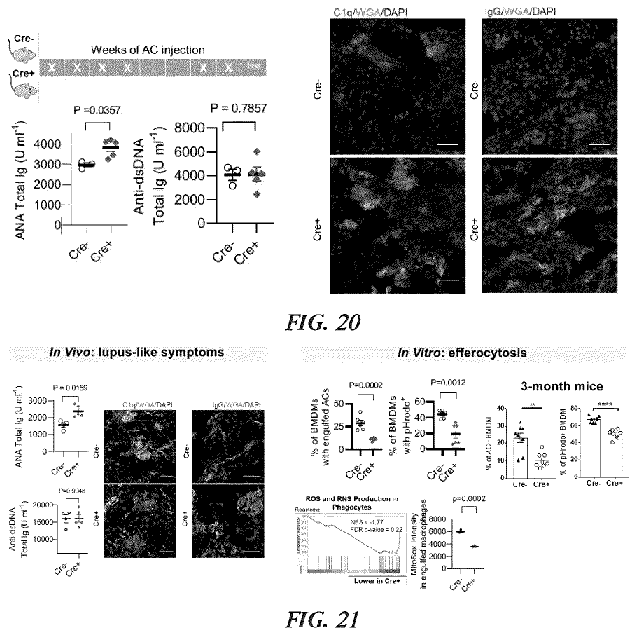

With a chronic AC injection (i.v. injection of 1×10\hat{ }7 apoptotic thymocytes at one time per week for 8 weeks), Myeloid-specific Wdfy3 knockout was sufficient to cause phenotypes mimicking lupus-like disorders, including increased ANA, deposition of C1q and IgG in the kidney, and increased serum IL-18 (FIG. 19). hWDFY3^{Tg} showed enhanced uptake and degradation of ACs in BMDMs (FIG. 11B) and increased efferocytic clearance by peritoneal macrophages in vivo (FIG. 4C). hWDFY3^{Tg} mice with chronic injection of AC showed lower anti-ANA and anti-dsDNA compared with control mice (FIG. 20), supporting the protective role of WDFY3 overexpression. It was therefore hypothesized that enhancing WDFY3 can have protective effects in lupus and lupus nephritis.

There is also clinical data supporting that lupus patients demonstrate impaired efferocytosis of dying cells by phagocytes and accumulation of nuclear materials that trigger inflammatory responses (Herrmann et al., 1998; Baumann et al., 2002; Huang et al., 2016; Geng et al., 2022). Therefore, both improved uptake and degradation are potentially beneficial in lupus (Jung & Suh, 2015; Mahajan et al., 2016; Abdolmaleki et al., 2018; Leventhal & Ross, 2016). In situ efferocytosis index is assessed in the kidney, kidney pathology, inflammation, and function, and systemic effects on autoimmunity, including autoantibody and inflammatory cytokines/chemokines, and splenomegaly.

Provided herein is the use of the pristane-induced lupus model. The model is commonly used and thoroughly characterized (Moore et al., 2021). Mice on C57BL/6J background develop autoimmunity and lupus nephritis (relatively modest but well established) (Moore et al., 2021), allowing the proposed research to leverage the Wdfy3 knockout and hWDFY3^{Tg} mice both on C57BL/6J background. The LysMCre mice are chosen because of their high efficiency in myeloid cell targeting (Shi et al., 2018; Abram et al., 2014). Since LysMCre also targets neutrophils (Shi et al., 2018; Abram et al., 2014), in order to specifically dissect the effects of monocytes/macrophages, and to assess the translational potential, adoptive monocyte transfer will also be used to pristane-treat wild-type C57BL/6J mice.

Wdfy3 knockout exacerbates while overexpression protects against lupus and lupus nephritis using the pristane-induced lupus mouse model.

### Experimental Models:

Mice models: A lupus mouse model that relies on the alkane pristane (2,6,10,14-tetramethylpentadecane) (Li et al., 2017; Moore et al., 2021) is employed. When injected into the peritoneal cavity, the naturally occurring hydrocarbon oil pristane induces chronic peritonitis accompanied by the production of autoantibodies directed against DNA and RNA-associated autoantigens and chronic inflammation, resulting in a disease closely resembling and meeting the classification criteria of lupus (Li et al., 2017; Moore et al., 2021). Susceptibility to pristane-induced lupus shows a female prevalence and is widespread among commonly used mouse strains, with diverse kinds of organ manifestation depending on the genetic background (Geng et al., 2022; Li et al., 2017; Moore et al., 2021; Reeves et al., 2009; Leiss et al., 2013). Mice on the C57BL/6 background develop comparably mild kidney disease (Leiss et al., 2013). Pristane also triggers apoptosis and necrosis. Environmental exposure to pristane is associated with the development of lupus in humans (Leiss et al., 2013). Pristane-induced lupus is therefore an ideal model to investigate the development of lupus-like autoimmunity and lupus nephritis (Leiss et al., 2013). Both male and female mice at about 2 months old are injected by a single intraperitoneal injection of 500 ul pristane (Sigma) and followed over 6 months then euthanized (Geng et al., 2022). Serum is collected before injection of pristane and in monthly intervals during the 6 months. Myeloid-specific Wdfy3 knockout and controls, and myeloid-specific hWDFY3^{Tg} and controls are used (12 mice per group and both male and female mice will be studied as described in C3 for power analysis and sex as a biological variable).

Adoptive transfer: In order to further establish the therapeutic premise and also to isolate the contribution of monocyte/macrophage WDFY3, a pristane-induced lupus model is established in wild-type C57BL/6J mice, i.e., CD45.1 C57BL/6J mice (B6.SJL-Ptprc^{a} Pepc^{b}/BoyJ, JAX #002014). Wild-type mice are injected with pristane at 2 months. Donor mice are myeloid-specific Wdfy3 knockout and controls, and myeloid-specific hWDFY3^{Tg} and controls. Donor BM-monocytes are isolated using anti-CD11b magnetic beads and LS column in a MidiMACS™ Separator attached to a magnetic MultiStand (Kanamaru et al., 2015; de Souza et al., 2019; Laoui et al., 2019). 1×10\hat{ }6 BM-monocytes are injected into the recipient mice (i.v.) weekly either at 2 months (the same time as pristane induction) or at 5 months (i.e., 3 months after pristane treatment when the mice already show lupus-like disorders thus mimicking the effects of monocyte transfer upon the establishment of lupus pathogenesis. The use of CD45.1 C57BL/6J allows the validation of donor monocyte engraftment by FACS because all donor mice strain express CD45.2.

### Experimental Design

Does myeloid-specific Wdfy3 knockout exacerbates lupus-like disorders? It is determined if compared with the respective controls, pristane-treated myeloid-specific Wdfy3 knockout mice or pristane-treated wild-type mice with adoptive transfer of Wdfy3 knockout BM-monocytes show: (1) poorer kidney in situ efferocytosis; (2) aggravated glomerulonephritis with increased glomerular scores, and increased glomerular deposition of IgG and complement C3; (3) elevated urine protein and serum creatinine; (4) increased kidney macrophage inflammation; (5) systemic lupus-like manifestation including increased serum autoantibodies and cytokines/chemokine, increased splenomegaly with higher spleen weight.

Does enhancing myeloid WDFY3 protects against lupus-like disorders? It is determined if compared with the respective controls, pristane-treated myeloid-specific hWDFY3^{Tg} mice or pristane-treated wild-type mice with adoptive transfer of hWDFY3^{Tg} BM-monocytes show: (1) improved kidney in situ efferocytosis; (2) improved glomerulonephritis with lower glomerular scores, and lower glomerular deposition of IgG and complement C3; (3) lower urine protein and serum creatinine; (4) ameliorated kidney macrophage inflammation; (5) reduced systemic lupus-like manifestation including increased serum autoantibodies and cytokines/chemokine, improved splenomegaly with lower spleen weight.

Kidney in situ efferocytosis: Kidney frozen sections are stained for TUNEL (for AC) and F4/80 (macrophage) and quantified for the ratio of macrophage-associated AC vs. AC not in proximity with macrophages as described (Cai et al., 2019; Kawabori et al., 2015). A lower ratio indicates impaired efferocytosis. The method has been widely used to determine defective efferocytosis in murine and human atherosclerosis plaques (Kolodgie et al., 2000; Shrijvers et al., 2005), and in brain injury (Cai et al., 2019; Kawabori et al., 2015).

Kidney pathology (Geng et al., 2022; Westerterp et al., 2017): Deposition of IgG and complement C3 in glomeruli are determined by IHC staining; periodic acid-Schiff (PAS) staining will be used to determine enlarged and hypercellular glomeruli, and mesangial expansion. For assessing glomerular immune and complement deposits, mice are perfused with PBS to minimize background. One kidney/mouse is embedded in OCT Tissue-Tek compound, snap-frozen, and. cryosectioned (5 μm). Sections will be fixed with acetone and stained with FITC-labeled antibodies against murine IgG (BioLegend #405305) or complement C3 (Cedarlane #CL7503F). Deposits are scored by 2 blinded individuals imaged by fluorescent microscopy, utilizing a semiquantitative scoring system as described previously; scored staining intensity by 0 (no staining), 1 (weak staining), 2 (positive staining), and 3 (strongly positive). The other kidney is fixed in 10% phosphate buffered formalin and embedded in paraffin. The 5-μm sections will be stained with PAS and then imaged with Leica SCN400 whole slide digital imaging (available at the Histology Core) and scored at a scale of 1-4 as described (Campbell et al., 2012). Ten glomeruli/section are analyzed for each mouse and the median per mouse is used for statistical analyses.

Kidney function (Geng et al., 2022): Urine mouse albumin are detected using the Mouse Albumin ELISA kit (Bethyl laboratories #E99-134). Serum creatinine will be measured using Creatinine Assay Kit (Abcam ab65340).

Kidney inflammation: kidney is dissociated with enzymatic digestion and macrophages will be labeled for CD11b and F4/80 and sorted by FACS (Nordlohne et al., 2021). RNA-seq is performed for kidney macrophages and data analyzed as described herein.

Systemic manifestations: Total IgG, IgG against dsDNA, histone, and sm/RNP in the serum is measured by ELISA (R&D). Plasma levels of inflammatory cytokines and chemokines previously reported as associated with structural renal damage and/or promote autoantibody production is profiled by using Cytokine & Chemokine Convenience 26-Plex Mouse ProcartaPlex™ Panel 1 (ThermoFisher), including IL-12, IL-6, MCP-1, IFN-α, IFN-g, TNFa, IL-18, IL1b, GROa, IL-17A, IL-2, IL-23, MIP-1a, MIP-1b, RANTES, etc. (Wong et al., 2002; Maczynska et al., 2006; Vila et al., 2007; Mohammad et al., 2015; Larosa et al., 2019; Oke et al., 2019; Xiang 2021; Zeng et al., 2021). Promising results by Luminex are validated by ELISA. Splenomegaly are assessed by spleen weight (Geng et al., 2022).

### Results

Wdfy3 deficiency likely exacerbates autoimmunity, proliferative glomerulonephritis and proteinuria, and kidney and systemic inflammation induced by pristane. WDFY3 overexpression likely protects pristane-induced lupus and lupus nephritis. Adoptive monocyte transfer of knockout or overexpression monocytes exerts consistent results, i.e., knockout exacerbates while overexpression protects lupus-like disorders, supporting the translational potential of targeting WDFY3-mediated efferocytosis in lupus.

### Alternative Strategies:

There are multiple Cre mouse lines used by the macrophage biology community (Shi et al., 2018; Abram et al., 2014). The Cx3cr1Cre targets monocytes/macrophages and not neutrophils, but editing was also seen in mast cells and cDCs (Shi et al., 2018; Abram et al., 2014). The Csf1rCre leaks to neutrophils, dendritic and T cells (Shi et al., 2018; Abram et al., 2014). The LysMCre in general shows the highest efficiency but induces recombination in neutrophils (Shi et al., 2018; Abram et al., 2014).

The pristane-induced model was selected because the model is widely used and thoroughly characterized, and also allows for directly leveraging Wdfy3 mouse models for proof of concept for new therapeutic strategies (Leiss et al., 2013). There are other mouse models for lupus studies, including spontaneous models of lupus, transgenic-induced lupus, gene knockout-induced lupus, and humanized mouse models of lupus (Li et al., 2017; Moore et al., 2021), which can be used. c) Data support the protective role of WDFY3 in lupus.

### Example 3

### WDFY3 is a Regulator of Efferocytosis Required for Both the Uptake and Degradation of Dying Cells by Macrophages

Defective clearance of apoptotic cells (ACs) by macrophages (efferocytosis) contributes to unresolved inflammation. A genome wide CRISPR screen (Example 1) discovered WDFY3 as a novel regulator required for the efficient uptake of ACs during efferocytosis. WDFY3 is well known as a scaffold protein facilitating the autophagic degradation of aggregated proteins in neurons. Yet, if and how WDFY3 may also be required for the efficient degradation of engulfed ACs by macrophages have not been characterized. This study thus aims to tackle the molecular mechanisms by which WDFY3 regulates the update and degradation of ACs by macrophages during efferocytosis.

Bone marrow-derived macrophages (BMDMs), peritoneal macrophages (PMs), and mice with myeloid-specific Wdfy3 knockout (LysMCre^{+/-}Wdfy3^{fl/fl}) or transgenic overexpression of human WDFY3 (hWDFY3^{Tg}) were used for in vitro and in vivo assays.

Effective uptake of ACs requires synchronized actin polymerization and depolymerization. Wdfy3 knockout BMDMs and PMs showed impaired uptake of ACs due to defective actin depolymerization, while binding was not affected. Wdfy3 knockout BMDMs and PMs also showed impaired lysosomal acidification and degradation of engulfed ACs. Mechanistically, Wdfy3 knockout impedes efficient LC3 lipidation upon AC engulfment, the last step required for phagosome-lysosome fusion and subsequent lysosomal degradation. Pull-down assay supports that WDFY3 interacts with GABARAP, but not LC3B, to mediate LC3 lipidation, supporting the direct involvement of WDFY3 protein in a process known as LC3-associated phagocytosis (LAP). The C-terminus of WDFY3 was sufficient to rescue impaired degradation, yet full-length WDFY3 is still required for regulating uptake. Intriguingly, BMDMs and PMs from hWDFY3^{Tg} mice showed enhanced AC uptake and degradation both in vitro and in vivo, suggesting that WDFY3 has a wide dynamic range in its regulatory capacity.

A role of WDFY3 was identified in the efficient uptake and degradation of ACs during macrophage efferocytosis. Enhancing WDFY3 is a therapeutic approach to promote the resolution of inflammation in diseases due to defective efferocytosis.

FIG. 20 shows myeloid-specific Wdfy3 knockout led to lupus-like pathological changes after chronic AC injection.

FIG. 21 illustrates myeloid-specific Wdfy3 knockout led to exacerbation of impaired efferocytosis and lupus-like pathological changes in 8-month mice.

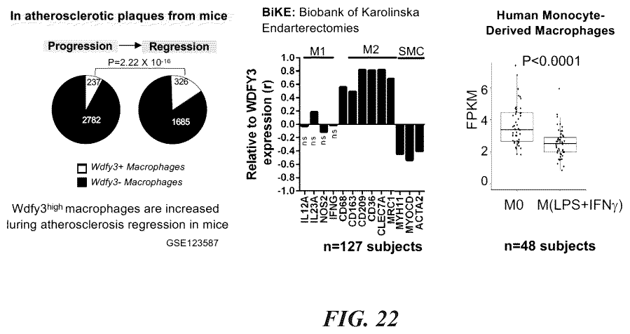

FIG. 22 illustrates that WDFY3^{hi} macrophage are increased during atherosclerotic regression in mice.

### BIBLIOGRAPHY

Abdolmaleki et al., Front Immunol., 9:1645 (2018).
Aber et al., Cell Rep., 41(3):111480 (2022),
Abernathy & Yoo, Cell Tissue Res., 359:179 (2015).
Abernathy et al., Cell Stem Cell., 21:332 (2017).
Abram et al., J. Immunol. Methods, 408:89 (2014).
Abudu et al., Autophagy., 15(10):1845-1847 (2019).
Advani et al., N. Engl. J. Med., 379:1711 (2018).
Andreu et al., Sci. Rep., 7:42225 (2017).
Andrew et al., Nat. Genet., 4:398 (1993).
Banez-Coronel et al., Neuron, 88:667 (2015).
Baumann et al., Arthritis Rheum., 46:191 (2002).
Beningo & Wang, J. Cell Sci., 115:849 (2002).
Bi et al., Arterioscler. Thromb. Vasc. Biol., 42:1139 (2022).
Birk et al., Journal of Cell Science, 126:1604 (2013).
Boada-Romero et al., Nat. Rev. Mol. Cell Biol., _: _ (2020).
Bolte & Cordelières, J. Microsc., 224:213 (2006).
Briggs & Sacks, EMBO Rep., 4:571 (2003).
Cai et al., Cell, 134:828 (2008).
Cai et al., JCI Insight, 4: _ (2019).
Campbell et al., Sci. Transl. Med., 4: 157ra141 (2012).
Carvalho et al., Philos. Trans. R. Soc. Lond. B. Biol. Sci., 368:20130005 (2013).
Chandra et al., Nat. Commun., 10:1528 (2019).
Chang et al., J. Clin. Investig., 128:607 (2018).
Chen et al., Bioinforma., 34:4095 (2018).
Chen et al., Circulation Research, 125:1087 (2019).
Cho et al., Nat. Protoc., 15:3971 (2020).
Choi et al., Curr. Rheumatol. Rep., 18:66 (2016).
Choi et al., Sci. Transl. Med., 12: _ (2020).
Chow et al., Sample Size Calculations in Clinical Research: Chapman & Hall/CRC Biostatistics Series; 2008.
Christian et al., Am. Heart J., 164:846 (2012).
Cinesi et al., Nat. Commun., 7:13272 (2016).
Clausen et al., Autophagy, 6:330 (2010).
Cornaby et al., Immunohorizons., 4:319 (2020).
Cremona et al., Nat. Neurosci., 14:469 (2011).
Croce & Yamamoto, Neurobiol. Dis., (2018).
Cunha et al., Cell, 175:429 (2018).
Curtis et al., Proc. Natl. Acad. Sci. U.S.A., 112:7231 (2015).
Dagda et al., J. Biol. Chem., 284:13843 (2009).
de Souza et al., Sci. Rep., 9:6434 (2019).
Deretic, Developmental Cell, 18:694 (2010).
Deva Magendhra Rao A K, et al., Mol Oncol. 2019 June; 13 (6):1342-1355.
DiFiglia et al., Proc. Natl. Acad. Sci. U.S.A., 104:17204 (2007).
Doench et al., Nat. Biotechnol., 34:184 (2016).
Doench, Nat. Rev. Genet., 19:67 (2018).
Doran et al., Nat. Rev. Immunol., _: _ (2019).
Dragich et al., eLife, 5: _ (2016).
Dragich et al., eLife, 5:14810 (2016).
Dragich et al., Society for Neuroscience, San Diego, CA (2013).
Dukare & Klempnauer, Biochim. Biophys. Acta., 1859:914 (2016).
Eenjes et al., J. Cell Sci., 129:1260 (2016).
Fan et al., PLOS Genet., 16:e1008786 (2020).
Fang et al., Aging Cell, 17:e12765 (2018).
Ferguson et al., Circulation Genomic and Precision Medicine, 11:e001907 (2018).
Ferrante, Biochim. Biophys. Acta., 1792:506 (2009).
Filimonenko et al., J. Cell Biol., 179 (3):485 (2008).
Filimonenko et al., J. Cell. Biol., 179:485 (2007).
Filimonenko et al., Mol. Cell, 38:265 (2010).
Fox & Yamamoto, “Macroautophagy of aggregation-prone proteins in neurodegenerative disease” ed. Hayat, M. A. AUTOPHAGY: Cancer, Other Pathologies, Inflammation, Immunity, and Infection. Vol. 6. Academic Press, San Diego, CA., 2015
Fox et al., Neuron, 105:813 (2020).
Fujita et al., Mol. Biol. Cell, 19:4651 (2008).
Gaipl et al., J. Autoimmun., 28:114 (2007).
Gayan et al., Genet. Epidemiol., 32:445 (2008).
Geng et al., Ann. Rheum. Dis., 81:255 (2022).
Gholamin et al., Sci. Transl. Med., 9: _ (2017).
Gomes, C. M. Methods in Molecular Biology: Protein Misfolding Diseases. Springer Protocols. Springer Journals, 2018.
Gray et al., J. Neurosci., 28:6182 (2008).
Green et al., Cell Death Differ., 23:915 (2016).
Greenlee-Wacker, Immunol. Rev., 273:357 (2016).
Hahn et al., Arthritis Care Res. (Hoboken), 64:797 (2012).
Hakala et al., Nat. Cell. Biol., 23:147 (2021).
Han et al., In Vitro Cell. Dev. Biol. Anim., 51:249 (2015).
Hanada et al., J. Biol. Chem., 282:37298 (2007).
Haney et al., Nat. Genet., 50:1716 (2018).
Harper et al., Proc. Natl. Acad. Sci. U.S.A., 102:5820 (2005).
Harper, Huntington's Disease, (W.B. Saunders Company Ltd., London, U.K., 1996).
Hayat et al., Cell Oncol. (Dordr.), 43:19 (2020).
Heckmann & Green, J. Cell Sci., 132: _ (2019).
Heckmann et al., J. Mol. Biol., 429:3561 (2017).
Hedgecock et al., Science, 220:1277 (1983).
Herrmann et al., Arthritis Rheum., 41:1241 (1998).
Hoji-Kawata et al., Nature, 494:201 (2013).
Huang et al., Int. J. Rheum. Dis., 19:1310 (2016).
Itakura & Mizushima, Autophagy, 6:764 (2010).
Iwata et al., J. Biol. Chem., 280:40282 (2005b).
Iwata et al., Proc. Natl. Acad. Sci. U.S.A., 102:13135 (2005a).
Jarr et al., N. Engl. J. Med., 384:382 (2021).
Jaumouillé & Waterman, Front. Immunol., 11:1097 (2020).
Jaumouillé et al., Nat. Cell Biol., 21:1357 (2019).
Jeong et al., Cell, 137(1):60 (2009).
Johnson et al., Fut. Med. Chem., 4(13):1715 (2012).
Jongstra-Bilen et al., J. Cell. Biol., 118:1443 (1992).
Joseph et al., iScience, 25:104184 (2022).
Joung et al., Nat. Protoc., 12:828 (2017).
Jung & Suh, International Journal of Rheumatic Diseases, 18:294 (2015).
Kamber et al., Nature, 597:549 (2021).
Kanamaru et al., Brain Res., 1605:49 (2015).
Kasikara et al., J. Clin. Invest., 131: _ (2021).
Kawabori et al., J. Neurosci., 35:3384 (2015).
Kirkin et al., Molecular Cell., 33:505 (2009).
Kirkin et al., Molecular Cell., 34:259 (2009).
Kojima et al., Circulation, 135:476 (2017).
Kojima et al., Nature, 536:86 (2016).
Kolodgie et al., Am. J. Pathol., 157:1259 (2000).
Korac et al., J. Cell. Sci., 126:580 (2013).
Kuma & Mizushima, Autophagy, 4:61 (2008).
Kuo et al., Acta Neuropathol., 125:879 (2013).
Laoui et al., Bio. Protoc., 9:e3134 (2019).
Larosa et al., Exp. Biol. Med. (Maywood), 244:42 (2019).
Lastres-Becker et al., Brain Res., 929 (2):236 (2002).
Lech & Anders, J. Am. Soc. Nephrol., 24:1357 (2013).
Leiss et al., Lupus, 22:778 (2013).
Lerm et al., J. Immunol., 178:7357 (2007).
Leventhal & Ross, Kidney Int., 90:238 (2016).
Li et al., Arterioscler. Thromb. Vasc. Biol., 39:e188 (2019).
Li et al., Crit. Rev. Immunol., 36:75 (2016).
Li et al., Curr. Opin. Rheumatol., 29:434 (2017).
Li et al., Genome Biol., 15:554 (2014).
Li et al., Genome Biol., 16:281 (2015).
Li et al., JCI Insight, 6: _ (2021).
Li et al., J. Cell. Physiol., 235:1141 (2020).
Lim & Yue, Dev. Cell., 32:491 (2015).
Lin et al., American Journal of Physiology Renal Physiology, 309: F901 (2015).
Lin et al., Arterioscler. Thromb. Vasc. Biol., 36:1434 (2016).
Liu et al., J. Cell. Biol., 216:1301 (2017).
Liu et al., Pathol. Oncol. Res., 26:1029 (2020).
Liu et al., Ther. Adv. Musculoskelet. Dis., 5:210 (2013).
Love et al., Genome Biol., 15:550 (2014).
Lystad et al., EMBO Rep., 15:557 (2014).
Ma & Morel, Front Immunol., 13:919792 (2022).
MacDonald & Gusella, Curr. Opin. Neurobiol., 6:638 (1996).
Machida et al., Biochem. Biophys. Res. Commun., 343:190 (2006).
Maczynska et al., Immunol. Lett., 102:79 (2006).
Mahajan et al., Front Immunol., 7:35 (2016).
Martin-Aparicio et al., J. Neurosci., 21 (22):8772 (2001).
Martinez et al., Nat. Cell Biol., 17:893 (2015).
Martinez et al., Nature, 533:115 (2016).
Martinez et al., Proc. Natl Acad. Sci. U.S.A., 108:17396 (2011).
Martinez, Curr. Opin. Immunol., 55:54 (2018).
Martinez, Curr. Protoc. Cell Biol., 87:e104 (2020).
Masters & Buss, Proc. Natl. Acad. Sci. U.S.A., 114:1595 (2017).
Mateer et al., Cell Motil. Cytoskeleton, 58:231 (2004).
Maul et al., J. Cell. Biol., 160:399 (2003).
Mehrotra & Ravichandran, Nature Reviews Drug Discovery, _: _ (2022).
Menalled et al., J. Comp. Neurol., 465:11 (2003).
Menalled et al., PloS one, 7:e49838 (2012).
Mijaljica et al., Autophagy, 8:1701 (2012).
Mizushima et al., Mol. Biol. Cell, 15:1101 (2004).
Mohammad et al., Hum. Immunol., 76:724 (2015).
Moon et al., Nat. Commun., 11:5489 (2020).
Moore et al., Nat. Immunol., 22:683 (2021).
Moreau et al., Nat. Commun., 6:8045 (2015).
Morel, Nat. Rev. Rheumatol., 13:280 (2017).
Morel, Nat. Rev. Rheumatol., 6:348 (2010).
Morioka et al., Immunity, 50:1149 (2019).
Morley, Int. J. Cell. Biol., 2012:935173 (2012).
Muñoz et al., Arthritis Rheum., 60:1733 (2009).
Nakahashi-Oda et al., Sci. Immunol., 6:eabe7915 (2021).
Napoli et al., Sci. Rep., 8:11348 (2018).
Nath et al., Nat. Cell. Biol., 16 (5):415 (2014).
Nordlohne et al., Sci. Rep., 11:13251 (2021).
Nurnberg et al., Circulation Research, 118:586 (2016).
Odagiri et al., Acta. Neuropathologica, 124:173 (2012).
Oke et al., Arthritis Res. Ther., 21:107 (2019).
Orosco et al., Nat. Commun., 5:4692 (2014).
Pan et al., Circulation, 142:2060 (2020).
Pankiv et al., J. Biol. Chem., 282:24131 (2007).
Patro et al., Nat. Methods, 14:417 (2017).
Penberthy & Ravichandran, Immunol. Rev., 269:44 (2016).
Perry et al., J. Biomed. Biotechnol., 2011:271694 (2011).
Perry et al., Nat. Cell Biol., 21:1532 (2019).
Pizzo et al., Mol. Psych., 18 (7):824 (2013).
Platt et al., Cell, 159:440 (2014).
Pollard & Borisy, Cell., 112:453 (2003).
Pre et al., Plos One, 9 (7): e103418 (2014).
Princely Abudu et al., Developmental Cell, 49:509 (2019).
Puigdellívol et al., Cell. Rep., 37:110148 (2021).
Qin et al., Nat. Methods., 18:133 (2021).
Ramet et al., Nature, 416:644 (2002).
Raudvere et al., Nucleic Acids Res., 47:W191 (2019).
Ravikumar et al., Hum. Mol. Genet., 11:1107 (2002).
Ravikumar et al., Nat. Genet., 37:771 (2005).
Reeves et al., Trends Immunol., 30:455 (2009).
Reimand et al., Nat. Protoc., 14:482 (2019).
Reinhart et al., Traffic, 22:23 (2021).
Richner et al., Nature Protocols, 10:1543 (2015).
Richter et al., Proc. Natl. Acad. Sci. U.S.A., 113:4039 (2016).
Rodrigues de Bastos & Nagai, PLOS One, 15:e0232284 (2020).
Rohatgi et al., Cell., 97:221 (1999).
Sang et al., Prog. Mol. Biol. Transl. Sci., 105:321 (2012).
Santulli-Marotto et al., PloS One, 10:e0145078 (2015).
Schilling et al., Hum. Mol. Genet., 8:397 (1999).
Schläfli A M, et al., Sci Rep. 7 (1):12980 (2017).
Schlam et al., Nat. Commun., 6:8623 (2015).
Schlam et al., Nat. Commun., 6:8623 (2015).
Schrijvers et al., Arterioscler. Thromb. Vasc. Biol., 25:1256 (2005).
Sedlyarov et al., Cell Host Microbe, 23:766 (2018).
Shah et al., PloS One, 2019 12:e0169614 (2019).
Shekhova, PLOS Pathog., 16:e1008470 (2020).
Shi et al., bioRxiv., _: _ (2022a).
Shi et al., Current Protocols in Stem Cell Biology, 48:e74 (2019).
Shi et al., Methods Mol. Biol., 1784:263 (2018).
Shi et al., Sci. Transl. Med., _: _ (2022b).
Shoji-Kawata et al., Nature, 494:201 (2013).
Silva et al., Immunity, 27:585 (2007).
Simonsen et al., J. Cell Sci., 117:4239 (2004).
Snyder-Keller et al., J. Neuropathol. Exp. Neurol., 69:1078 (2010).
Soneson et al., F1000Res, 4:1521 (2015).
Søreng et al., Journal of Cell Science, 135: _ (2022).
Sosale et al., Blood, 125:542 (2015).
Southwell et al., J. Neurosci., 29:13589 (2009).
Steffan, Cell. Cycle, 9:3401 (2010).
Stromhaug et al., Biochem. J., 335 (Pt 2):217 (1998).
Subramanian et al., Proc. Natl Acad. Sci. U.S.A., 102:15545 (2005).
Svane et al., J. Chem. Phys., 144:084708 (2016).
Taha et al., Cell. Rep., 29:1010 (2019).
Takahashi et al., Nat. Cell Biol., 9:1142 (2007).
Tang et al., Neuron, 83 (5):1131 (2014).
Teng et al., Immunol. Rev., 295:167 (2020).
Thurston et al., Nature Immunology, 10:1215 (2009).
Trzeciak et al., Cell Metabolism, _: _ (2021).
Trzeciak et al., Cell. Metab., _: _ (2021).
Valente et al., Acta. Histochem., 119:315 (2017).
Vaquer-Alicea & Diamond, Annu. Rev. Biochem., 88:785 (2019).
Victor et al., Nat. Neurosci., 21:341 (2018).
Victor et al., Neuron, 84:311 (2014).
Vilá et al., Clin. Rheumatol., 26:718 (2007).
Vos et al., Hum. Mol. Genet., 19:4677 (2010).
Walker, Lancet., 369:218 (2007).
Wang et al., Cell, _: _ (2017).
Wang et al., J. Cell. Biol., 181:803 (2008).
Wang et al., Nat. Protoc., 14:756 (2019).
Wang et al., Science, 273:660 (1996).
Wartosch et al., Traffic, 16:727 (2015).
Weidberg & Elazar, Sci. Signal, 4: pe39 (2011).
Westerterp et al., Cell. Metab., 25:1294 (2017).
Wexler et al., Proc. Natl. Acad. Sci. U.S.A., 101:3498 (2004).
Wild et al., Science, 333:228 (2011).
Wong et al., Clin. Exp. Immunol., 130:345 (2002).
Wu, F., et al. Cancer Cell Int 18, 107 (2018).
Xia et al., Autophagy, 15:960 (2019).
Xiang et al., Sci. Rep., 11:4707 (2021).
Xu & Du, Front Cell Dev. Biol., 9:746818 (2021).
Xue et al., Physiological Genomics, 49:287 (2017).
Yamamoto & Simonsen, Autophagy, 7:346 (2011).
Yamamoto & Yue, Ann. Rev. Neurosci., 37:55 (2014).
Yamamoto et al., Cell, 101:57 (2000).
Yamamoto et al., J. Cell. Biol., 172:719 (2006).
Yamamoto et al., Neurobiol. Dis., 8(6):923 (2001).
Yang-Klingler et al., Springer Protocols. Springer Journals, (2018).
Yin & Heit, Frontiers in Immunology, 12: _ (2021).
Yin et al., Sci. Transl. Med., 7: 274ra18 (2015).
Yoo et al., Nature, 476:228 (2011).
Yu et al., Autophagy., 14:207 (2018).
Yu et al., J. Neurosci., 23:2193 (2003).
Yuan et al., J. Biol. Chem., 285:28953 (2010).
Yurdagul et al., Arterioscler. Thromb. Vasc. Biol., _: _ (2021).
Yurdagul et al., Front. Cardiovascular Med., 4:86 (2017).
Zeng et al., BMC Immunol., 22:82 (2021).
Zhang & Reilly, Arterioscler. Thromb. Vasc. Biol., 37:2000 (2017).
Zhang et al., Arterioscler. Thromb. Vasc. Biol., 37:2156 (2017).
Zhang et al., Circulation Res., 117:17 (2015).
Zhang et al., J. Cell Mol. Med., 24:8206 (2020).
Zhao et al., J. Leukoc. Biol., 102:1313 (2017).
Zheng et al., PLOS Genet., 6:e1000838 (2010).
Zhu et al., Bioinformatics, 35:2084 (2019).

### Example 4

### Introduction

Efficient efferocytosis of dying cells by phagocytes, mainly macrophages, is needed in maintaining homeostasis (1, 2). The role of apoptotic debris as an immunogenic stimulus in autoimmune disease has been recognized (1, 3). When efferocytosis fails, apoptotic cells (ACs) can rupture, releasing cellular materials that can evoke autoimmune responses (1). Dead cells not effectively cleared via efferocytosis release self-antigens. These autoantigens are processed and presented by antigen presenting cells, including macrophages, to CD4⁺ T cells that activate B cells to produce autoantibodies (4, 5). Autoantibodies deposit in organs, including kidney, to cause inflammatory responses and pathological changes. Excessive AC death and impaired efferocytosis are observed in patients with systemic lupus erythematosus (6-9). Experimental studies establish that mice lacking efferocytic receptors, such as Tim4 or Mertk, have impaired clearance of ACs and develop autoimmune-like phenotypes (10,11). However, it remains unclear whether the immunogenic autoantigens due to failed efferocytosis represent the sole culprit of increased autoimmunity or the cell autologous effects within the macrophage triggered by AC engulfment directly impact macrophage ability to activate T cells and autoimmune response. It has also never been established if enhancing macrophage efferocytosis can be protective in autoimmune like phenotypes.

Provided herein, it was identified, through a genome wide CRISPR screening, that WDFY3 is required for both the engulfment and the subsequent lysosomal acidification and degradation of ACs12. Here, the molecular mechanisms of myeloid deficiency of Wdfy3 in driving autoimmune responses and the therapeutic uses of overexpressing WDFY3 in enhancing efferocytosis and suppressing autoimmune responses is demonstrated. It was discovered that, in addition to its role in regulating efferocytosis, WDFY3 governs the antigen presentation capacity of macrophages, with knockout exacerbates, while overexpress suppresses MHC-II mediated antigen presentation for CD4⁺ T cell activation. WDFY3 also plays a cell intrinsic role in suppressing inflammasome activation induced by AC engulfment. The findings show that WDFY3 plays a pivotal role in modulating autoimmune responses through both efferocytosis dependent and cell intrinsic mechanisms by regulating macrophage-T cell crosstalk for T cell activation.

### Myeloid Knockout of Wdfy3 Exacerbates Autoimmunity Induced by Systemic AC Injections in Young Mice and Spontaneous Autoimmunity in Middle-Aged Mice

WDFY3 was identified as a positive regulator required for the efferocytic clearance of ACs, but dispensable for the phagocytic clearance of other cargos, such as beads, zymosan, or red blood cells (12). WDFY3 is needed for both the engulfment and the lysosomal acidification of the engulfed ACs for subsequent degradation (12). The role of WDFY3 in efferocytosis was established in vivo in mice with dexamethasone-mediated thymocyte apoptosis and efferocytosis by peritoneal macrophages, as well as in human monocyte-derived macrophages (HMDMs) (12).

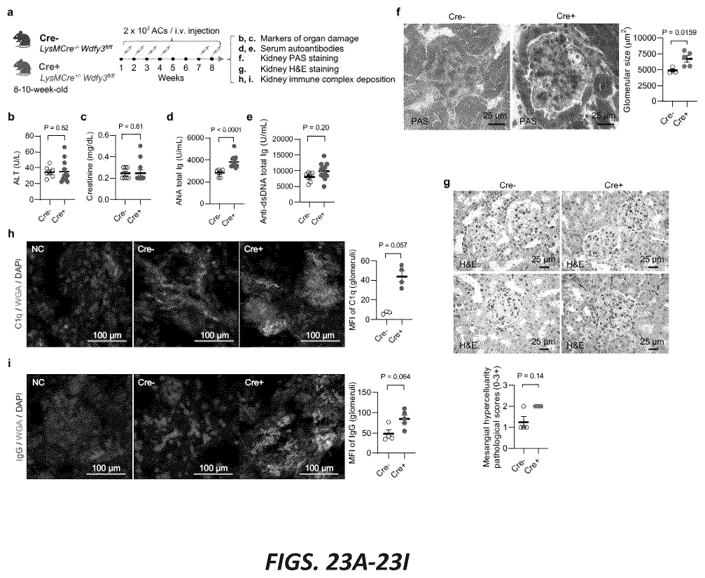

Given the important role of WDFY3 in regulating multiple steps of efferocytosis, the pathological consequence of impaired efferocytosis was determined with myeloid knockout of Wdfy3, including the major organ affected by increased AC burden. Whole-body triple knockout of Ax1, Tyro3 and Mertk led to enlarged spleen in mice beginning at ˜4 weeks that progressively develop autoimmune-like phenotypes (13). Cohen et al. demonstrated that whole-body knockout of Mertk in mice showed delayed clearance of exogenously injected ACs and progressive development of autoimmune phenotypes (10), though these were less severe compared to the triple knockout mice. These genes target the “binding” phase and, although they are highly expressed in myeloid cells, the effects observed in whole-body knockouts may be attributable to the knockout effects in other cell types. Therefore, the effects of myeloid-specific knockout of Wdfy3 were determined, which disrupts the “eating” phase of efferocytosis, on autoimmune-like phenotypes. Indeed, increasing burden of ACs elicits autoimmune-like phenotypes in young mice with myeloid Wdfy3 deficiency (FIG. 23), driven by LysMCre-mediated Cre⁻loxp recombination for myeloid-specific knockout of Wdfy3. Beginning at 8-10 weeks of age, Cre⁻ and Cre⁺ mice were injected with UV-irradiated apoptotic thymocytes over an 8-week period (FIG. 23a for schematics of experimental design). Markers of organ damage, including serum alanine aminotransferase (ALT) and creatinine (FIGS. 23b and 23c), were not different between Cre⁻ and Cre⁺ mice. Cre⁺ mice with AC injections showed higher serum antinuclear antibodies (ANA) (FIG. 23d), but not anti-dsDNAs (FIG. 23e) compared to Cre⁻ mice. Histologically, Cre⁺ mice exhibited more severe histological changes of glomerulonephritis-like phenotypes, including enlarged glomeruli area (FIG. 23f) and increased mesangial hypercellularity (FIG. 23g). Furthermore, Cre⁺ mice displayed increased C1q and IgG deposition along the mesangial areas in the kidneys than Cre⁻ mice (FIGS. 23h and 23i). It was further confirmed that the injected ACs are mostly accumulated in the spleen, with minimal accumulation in the kidney or liver, suggesting that spleen is directly involved in the clearance of ACs and triggering systemic immune responses (Fig S1). The results also implicate that kidney immune complex deposition and nephritis-like phenotype were unlikely due to the direct effects of AC accumulation. Instead, the increased AC burden and inefficient efferocytosis in Cre⁺ mice led to increased autoantigen-triggered autoantibody production, thereby the subsequent pathological changes in the kidney. Collectively, these data demonstrate that defective AC clearance associated with Wdfy3 deficiency can result in the development of autoimmune-like phenotypes, in particular, lupus-like nephritis.

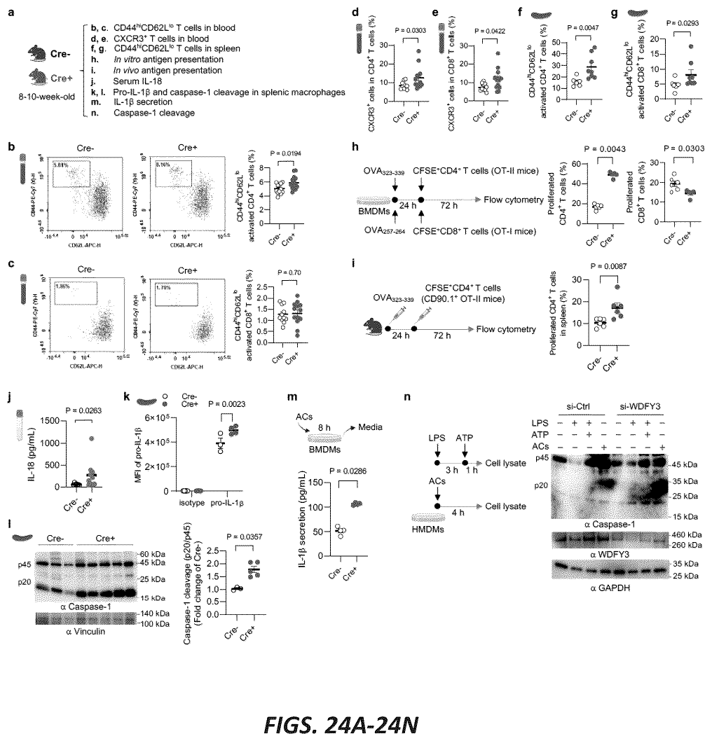

These autoimmune phenotypes were also observed in middle-aged Cre⁺ mice (52-60-week-old) spontaneously without AC injections (Fig S2), suggesting progressive autoimmune responses. It was first confirmed that BMDMs of middle-aged Cre⁺ mice show complete loss of WDFY3 protein (Fig S2b) and have impaired engulfment and acidification of the engulfed ACs (Fig S2c). Middle-aged Cre⁺ mice also showed increased spleen and kidney weight (Fig S2d) and increased serum ALT (Fig S2e), suggesting that chronic myeloid knockout of Wdfy3 led to progressive organ damage. Creatinine was comparable between Cre⁻ and Cre⁺ mice (Fig S2f), suggesting no functional kidney damage. Serum ANA was increased in Cre⁺ middle-aged mice (Fig S2g), together with increased glomerular size (FIG. 24i) and increased C1q and IgG immune complex deposition in the glomeruli (Fig S2j and S2k). Anti-dsDNA antibodies (Fig S2h) were not different between middle-aged Cre⁻ and Cre⁺ mice. These observations suggest that myeloid knockout of Wdfy3 causes an autoinflammatory, lupus-like syndrome in middle-aged mice, resembling the phenotypes observed in young mice with AC injections.

### Myeloid Deficiency of Wdfy3 Exacerbates Autoimmune Responses Via Efferocytosis-Dependent and -Independent Mechanisms, the Latter Involves Increased Antigen Presentation and Inflammasome Activation

The results confirmed that a myeloid-specific knockout of Wdfy3, which impairs efferocytosis, leads to autoimmune-like phenotypes in mice. However, it remains unclear whether the autoimmune response is solely due to the increased release of autoantigen from dead cells because of impaired clearance, or if the autologous effects within macrophages triggered by AC engulfment directly impact the macrophage's ability to activate T cells and provoke autoimmune responses.

In young mice without AC burden, Cre⁺ mice did not show changes in ALT, ANA, or pathological features and immune complex deposition in the kidney (Fig S3), suggesting that myeloid knockout of Wdfy3 was not sufficient to drive autoimmune-like disorders in young mice without enhanced AC burden. Myeloid knockout of Wdfy3 was, however, sufficient to lead to T cell activation in young mice (FIG. 24b-24g). Specifically, there was an increased percentage of CD44^{hi}CD621^{lo} activated T cells in CD4⁺ T cells, though not in CD8⁺ T cells, in the blood (FIGS. 24b and 24c). CXCR3 is a marker of Th1 cell activation, which increased in both CD4⁺ and CD8⁺ T cells in the blood (FIGS. 24d and 4e). The percentage of CD44^{hi}CD62L^{lo} activated T cells in CD4⁺ T cells and CD8⁺ T cells were also increased in the spleen (FIGS. 24f and 24g).

T cells are activated when their receptors recognize antigens presented by antigen-presenting cells, such as macrophages, dendritic cells, and B cells, and receive additional signals from cytokines (14-16). To determine if macrophage WDFY3 directly regulates antigen presentation capacity by macrophages via cell intrinsic mechanisms, a classical antigen presentation assay was performed using soluble antigens, which require the interaction of the MHC complex on ovalbumin (OVA)-treated macrophages with T cells isolated from OT-II or OT-I transgenic mice (FIG. 24h). The OT-II transgenic mice express mouse T cell receptors designed to specifically recognize OVA peptide residues 323-339 (OVA₃₂₃₋₃₃₉). CD4⁺ T cells of the OT-II mice primarily recognize OVA₃₂₃₋₃₃₉ when presented by the MHC-II molecule. The results show that myeloid knockout of Wdfy3 led to increased MHC-II mediated antigen presentation to CD4⁺ T cells (FIG. 24h). The OT-I transgenic mice express mouse T cell receptors designed to recognize OVA peptide residues 257-264 (OVA₂₅₇₋₂₆₄). CD8 T cells of the OT-I mice primarily recognize OVA₂₅₇₋₂₆₄ when presented by the MHC-I molecule. The results show that myeloid-knockout of Wdfy3 led to decreased MHC-I mediated antigen presentation to CD8⁺ T cells (FIG. 24h).

To confirm the findings in vivo, both Cre⁺ mice and their control counterparts were immunized with OVA₃₃₂₋₃₃₉, followed by an adoptive transplantation of CD4⁺ T cells from CD90.1⁺ OT-II mice, which specifically recognize OVA-MHC-II complexes. Discrimination of donor T cells from the recipient's T cells relies on the expression of CD90.1, a surface marker that is unique to the donor T cells, while the recipient mice express CD90.2. To monitor proliferation, these CD4⁺CD90.1⁺ OT-II T cells were labeled with carboxyfluorescein succinimidyl ester (CFSE). In Cre⁺ mice, there was a significant CFSE dilution in CD4⁺ CD90.1⁺OT-II T cells in the spleen (FIG. 24i), indicating enhanced antigen presentation by Wdfy3-deficient splenic macrophages that promotes T cell proliferation. The results suggest that WDFY3 deficiency regulates MHC-II mediated antigen presentation to the surface of macrophage, contributing to increased antigen presentation to CD4⁺ T cells and enhanced activation. In contrast, the activation of CD8⁺ T cells observed in young Cre⁺ mice was unlikely directly mediated by intrinsic mechanisms within macrophages.

It was also determined whether a myeloid knockout of Wdfy3 in young mice is sufficient to drive systemic inflammatory cytokine levels. A 26-Plex ProcartaPlex mouse cytokine and chemokine panel was used, which includes cytokines produced by activated T cells or those that instruct T cell activation (Fig S3). Through this initial screening, it was found that only IL-18 was increased in the serum of Cre⁺ mice (FIG. 24j). IL-18, together with IL-1β and IL-1α, are pro-inflammatory cytokines in the IL-1 family. Their secretion, especially the secretion of IL-1β and IL-18, is regulated by inflammasomes which are multi-protein complexes containing sensor proteins, adaptor protein and caspase-1. IL-1β and IL-18 are the direct indicator of inflammasome activation and caspase-1 is a key effector enzyme involved in the cleavage and activation of pro-IL-1β and pro-IL-18. It was then examined whether the Wdfy3 knockout affects inflammasome activation. Indeed, splenic F4/80⁺ macrophages of Cre⁺ mice show increased pro-IL-1b protein expression (FIG. 24k) and caspase-1 cleavage (FIG. 24l), suggesting increased priming and activation of the inflammasome.

To test whether Wdfy3 knockout in macrophages regulate inflammasome activation in the context of efferocytosis. IL-10 secretion in BMDMs co-incubated with ACs for 8 h was determined. Wdfy3-deficient BMDMs showed increased IL-10 secretion (FIG. 24m). To establish human relevance and that WDFY3 directly regulates inflammasome activation, the effects of siRNA mediated WDFY3 knockdown in HMDMs was determined. Either treatment with AC or LPS and ATP led to increased caspase-1 cleavage in HMDMs (FIG. 24n). WDFY3 knockdown further exacerbates caspase-1 cleavage, suggesting that WDFY3 plays a role in suppressing NLRP3 inflammasome activation.

IL-1β was known to enhance antigen-primed CD4⁺ and CD8⁺ T-cell activation (17). IL-18 and 1L-12 synergize for IFN-γ production in Th1 cells (18). Increased IL-12 or IFN-γ were not observed, suggesting that IL-18 did not directly contribute to T cell activation in young myeloid-specific Wdfy3 knockout mice. Taken together, myeloid Wdfy3 knockout leads to increased T cell activation likely via efferocytosis-dependent and -independent mechanisms. Wdfy3 deficiency leads to increase antigen presentation capacity and inflammasome activation in macrophages, which further exacerbates autoantigen-induced T cell activation.

### Overexpression of WDFY3 Enhanced Macrophage Efferocytosis In Vitro and In Vivo

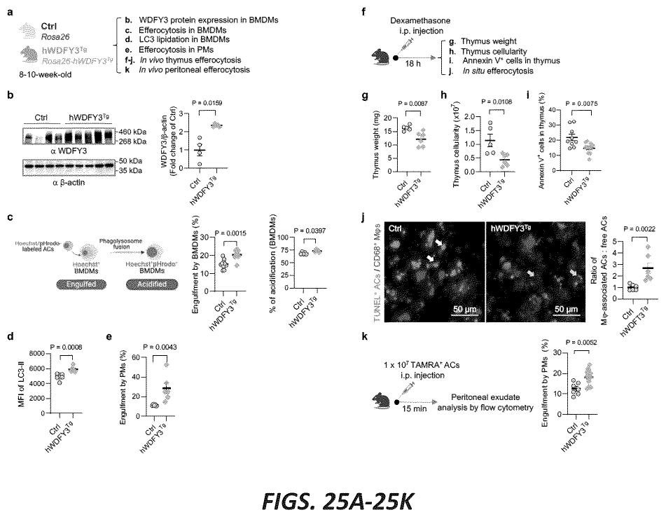

Given the role of WDFY3 in efferocytosis, it was investigated whether overexpressing WDFY3 could further enhance this process (FIG. 25). Successful overexpression was confirmed by Western blotting (FIG. 3b). BMDMs from mice with transgenic overexpression of human WDFY3 (hWDFY3^{T}9) showed higher engulfment capacity compared to their control counterparts (Ctrl), indicating increased efferocytosis (FIG. 25c). The percentage of engulfed ACs that were also acidified was slightly increased in WDFY3-overexpressing mice (FIG. 25c), consistent with increased LC3 lipidation (FIG. 25d), which is the final step required for phagosome-lysosome fusion and the subsequent lysosomal acidification (19). This increase suggests that the enhanced engulfment of cargo is associated with efficient degradation. Enhanced efferocytosis was also confirmed in peritoneal macrophages (PMs) (FIG. 25e).

Efferocytosis in mice was further validated using two in vivo assays: thymus efferocytosis and PM efferocytosis (12). The in vivo thymus efferocytosis assay involved injecting dexamethasone intraperitoneally into Ctrl and hWDFY3^{Tg} mice to induce thymocyte apoptosis (FIG. 25f), as the thymus is particularly sensitive to dexamethasone-induced apoptosis (20). After 18 hours, as expected, thymus weight and cellularity decreased in Ctrl mice following dexamethasone treatment due to thymocyte apoptosis and the efferocytic clearance of dead cells (FIGS. 25g and 25h). Compared to the Ctrl mice, hWDFY3^{Tg} mice showed significantly lower thymus weights and decreased cellularity, indicating enhanced efferocytic capacity. Consistently, thymi from hWDFY3^{Tg} mice also contained fewer Annexin V⁺ cells (FIG. 25i). In situ efferocytosis was next assessed using TUNEL staining on thymus sections to calculate the ratio of TUNEL⁺ cells associate with CD68⁺ macrophages and “free” TUNEL⁺ cells not in proximity to macrophages (FIG. 25j). The data showed an increased ratio of macrophage associated AC: free AC in hWDFY3^{Tg} mice, indicating increased efferocytosis in the thymus. The findings were further confirmed by assessing efferocytosis by PMs in vivo. Peritoneal exudate was collected 15 mins after injecting TAMRA-labeled ACs. PMs from hWDFY3^{Tg} mice showed an increased percentage of TAMRA-labeled F4/80⁺ macrophages, indicating enhanced efferocytotic capacity in the hWDFY3^{Tg} mice (FIG. 25k).

### Overexpression of WDFY3 Attenuates Autoimmunity Induced by Systemic AC Injections in Young Mice and Spontaneous Autoimmunity in Middle-Aged Mice

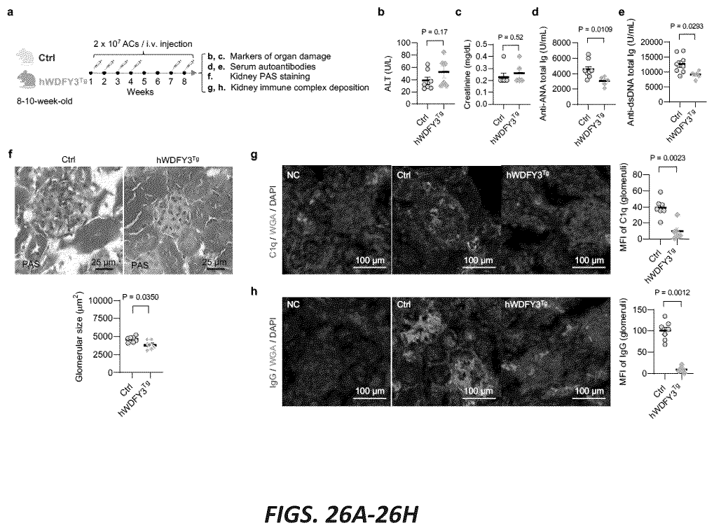

After confirming the enhanced efferocytotic capacity in macrophages overexpressing WDFY3, it was determined whether WDFY3 overexpression in mice provides protection against autoimmunity. hWDFY3^{Tg} mice were challenged with repeated injection of ACs (FIG. 26). Organ damage markers remain low and comparable between Ctrl and hWFY3^{Tg} mice (FIGS. 26b and 26c). However, compared to Ctrl mice, hWDFY3^{Tg} mice showed lower levels of ANA and ant-dsDNA (FIGS. 26d and 26e), smaller glomeruli (FIG. 26f), and reduced C1q and IgG deposition in the kidney (FIGS. 26g and 26h), indicating a protective effect from AC injections.

The protective effects of WDFY3 overexpression also extended to middle-aged mice without AC injections (Fig S4). It was first confirmed that middle-aged hWDFY3^{Tg} mice also had enhanced efferocytosis (Fig S4b). Middle-aged hWDFY3^{Tg} mice show lower kidney and lymph node weight (Fig S4c), lower ALT levels (Fig S4d), and reduced ANA and anti-dsDNA (Fig S4f and S4g), while creatinine level remained low and comparable (Fig S4e). Additionally, hWDFY3^{Tg} mice exhibited smaller glomeruli (Fig S4h) and lower IgG deposition in the kidney (Fig S4i).

Taken together, these findings highlight the use of WDFY3 overexpression as a therapeutic strategy for mitigating autoimmunity.

### Overexpression of WDFY3 Ameliorated T Cells Activation by Inhibiting Antigen Presentation and Inflammasome Activation

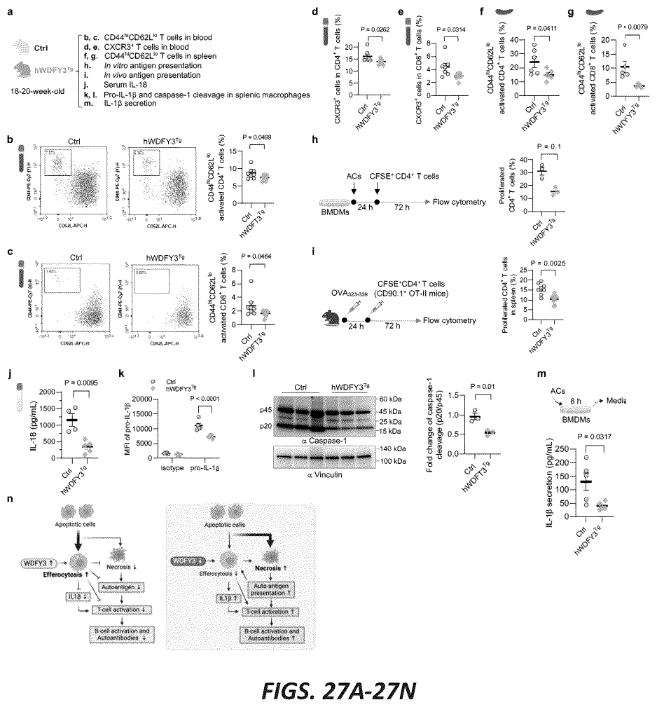

It was next examined whether WDFY3 overexpression suppresses T cell activation and inflammasome activation (FIG. 27). The percentage of CD44^{hi}CD62L^{lo} activated T cells in CD4⁺ and CD8⁺ T cells, as well as CXCR3⁺ activated T cells in CD4⁺ and CD8⁺ T cells in the blood, showed a modest reduction in hWDFY3Tg mice (FIG. 27b-27e). The percentage of CD44^{hi}CD62L^{lo} activated T cells in splenic CD4⁺ and CD8⁺ T cells also decreased in hWDFY3Tg mice (FIG. 27f-27g). An experiment was designed to directly assess efferocyte-T cell interaction modified from previous published protocols (21). ACs were co-cultured with BMDMs for 24 h to induced efferocytosis and then washed away, followed by the addition of CFSE-labeled CD4⁺ T cells to assess T cell proliferation over 72 h. Efferocytic BMDMs induced T cell activation, which was attenuated in BMDMs from hWDFY3Tg mice (FIG. 27h). Moreover, hWDFY3Tg mice also showed enhanced MHC-II mediated antigen presentation to CD4⁺ T cells in vivo (FIG. 27i). Taken together, WDFY3 overexpression suppresses MHC-II mediated antigen presentation and efferocyte-T cell crosstalk, thereby attenuating T cell activation.

Lower serum IL-18 levels were also observed in 15-month-old hWDFY3^{Tg} mice compared to Ctrl (FIG. 27j). The hWDFY3^{Tg} mice showed decreased pro-IL-10 expression and caspase-1 cleavage in splenic macrophages (FIGS. 27k and 27l). Additionally, AC-induced IL-103 secretion was lower in hWDFY3^{Tg} mice (FIG. 27m), suggesting that WDFY3 overexpression suppressed AC-induced inflammasome activation.

Mechanistically, it was speculated that, WDFY3 exerts its functional roles through protein-protein interaction. This hypothesis arises from RNA-seq analysis of BMDMs from Ctrl and hWDFY3^{Tg} mice, which revealed few differentially expressed genes (Fig S5a). This suggests that overexpression of WDFY3 has no significant effects at the transcriptomic level in BMDMs. To identify protein-protein interaction partners of macrophage WDFY3, TurboID-based proximity labeling was performed (22, 23) (Fig S6). TurboID is an engineered biotin ligase that enables fast biotinylation of proximal proteins, which can later be isolated and identified using streptavidin affinity purification followed by mass spectrometry (22,23). Lentiviral vectors expressing TurboID fused to C-terminal WDFY3₍₂₅₄₃₋₃₅₂₆₎ were transduced into BMDMs. The C-terminal WDFY3 was used due to the technical challenge of packaging the full-length WDFY3 cDNA, which is 10.8 kb, thus preventing effective transfection or transduction. Additionally, the C-terminal WDFY contains known functional domains (24), including PH-BEACH, WD40, LIR, and FYVE domains. Proximity labeling identified proximal proteins of C-terminal WDFY3, including proteins in the Arp2/3 complex and myosins that regulate cytoskeleton remodeling (Fig S5b). The role of cytoskeleton network in regulating macrophage antigen presentation is well recognized. Myosin 1e and n-cofilin (25) are needed for antigen presentation through the MHCII complex. F-actin interacts with activated NLRP3 inflammasome (26). There is evidence that actin polymerization suppresses, while aberrant actin depolymerization triggers, inflammasome activation (26-28). WDFY3 may govern antigen presentation and inflammasome activation via its interaction and fine-tuning of F-actin and cytoskeleton dynamics.

Notably, WDFY4, a paralog of WDFY3, is highly expressed in B cells and dendritic cells among all immune cells and is associated with increased risks of systemic lupus erythematosus and rheumatoid arthritis by genome-wide association studies. WDFY4 is needed for cross-presentation by cDC1 cells (15). In contrast, WDFY3 is abundantly expressed in monocytes and macrophages, with low expression in B cells and dendritic cells (Fig S7). Given the high homology between WDFY3 and WDFY4, it is speculated that both proteins share similar molecular mechanisms and protein binding partners. This is supported by pull-down analyses of WDFY4, which reveal its interaction with cytoskeleton proteins.

### BIBLIOGRAPHY

1 Doran, A. C., Yurdagul, A., Jr. & Tabas, I. Efferocytosis in health and disease. Nature reviews. Immunology (2019). doi.org:10.1038/s41577-019-0240-6
2 Boada-Romero, E., Martinez, J., Heckmann, B. L. & Green, D. R. The clearance of dead cells by efferocytosis. Nat Rev Mol Cell Biol (2020). doi.org:10.1038/s41580-020-0232-1
3 Mehrotra, P. & Ravichandran, K. S. Drugging the efferocytosis process: concepts and opportunities. Nature reviews. Drug discovery (2022). doi.org:10.1038/s41573-022-00470-y
4 Sachet, M., Liang, Y. Y. & Oehler, R. The immune response to secondary necrotic cells. Apoptosis 22, 1189-1204 (2017). doi.org:10.1007/s10495-017-1413-z
5 Kawano, M. & Nagata, S. Efferocytosis and autoimmune disease. Int Immunol 30, 551-558 (2018). doi.org:10.1093/intimm/dxy055
6 Herrmann, M. et al. Impaired phagocytosis of apoptotic cell material by monocyte-derived macrophages from patients with systemic lupus erythematosus. Arthritis Rheum 41, 1241-1250 (1998). doi.org:10.1002/1529-0131(199807)41:7<1241:Aid-art15>3.0.Co; 2-h
7 Baumann, I. et al. Impaired uptake of apoptotic cells into tingible body macrophages in germinal centers of patients with systemic lupus erythematosus. Arthritis Rheum 46, 191-201 (2002). doi.org:10.1002/1529-0131(200201)46:1<191:Aid-art10027>3.0.Co;2-k
8 Muñoz, L. E. et al. Remnants of secondarily necrotic cells fuel inflammation in systemic lupus erythematosus. Arthritis Rheum 60, 1733-1742 (2009). doi.org:10.1002/art.24535
9 Huang, W. N., Tso, T. K., Wu, H. C., Yang, H. F. & Tsay, G. J. Impaired phagocytosis of apoptotic cell material in serologically active clinically quiescent patients with systemic lupus erythematosis. Int J Rheum Dis 19, 1310-1316 (2016). doi.org:10.1111/1756-185x.12826
10 Cohen, P. L. et al. Delayed apoptotic cell clearance and lupus-like autoimmunity in mice lacking the c-mer membrane tyrosine kinase. The Journal of experimental medicine 196, 135-140 (2002). doi.org:10.1084/jem.20012094
11 Rodriguez-Manzanet, R. et al. T and B cell hyperactivity and autoimmunity associated with niche specific defects in apoptotic body clearance in TIM-4-deficient mice. Proceedings of the National Academy of Sciences of the United States of America 107, 8706-8711 (2010). doi.org:10.1073/pnas.0910359107
12 Shi, J. et al. A genome-wide CRISPR screen identifies WDFY3 as a regulator of macrophage efferocytosis. Nature communications 13, 7929 (2022). doi.org:10.1038/s41467-022-35604-8
13 Lu, Q. & Lemke, G. Homeostatic regulation of the immune system by receptor tyrosine kinases of the Tyro 3 family. Science (New York, N.Y.) 293, 306-311 (2001). doi.org:10.1 126/science.1061663
14 Westerterp, M. et al. Cholesterol Accumulation in Dendritic Cells Links the Inflammasome to Acquired Immunity. Cell metabolism 25, 1294-1304.e 1296 (2017). doi.org:10.1016/j.cmet.2017.04.005
15 Theisen, D. J. et al. WDFY4 is required for cross-presentation in response to viral and tumor antigens. Science (New York, N.Y.) 362, 694-699 (2018). doi.org:10.1126/science.aat5030
16 Guerriero, J. L. Macrophages: Their Untold Story in T Cell Activation and Function. Int Rev Cell Mol Biol 342, 73-93 (2019). doi.org:10.1016/bs.ircmb.2018.07.001
17 Ben-Sasson, S. Z., Wang, K., Cohen, J. & Paul, W. E. IL-103 strikingly enhances antigen-driven CD4 and CD8 T-cell responses. Cold Spring Harb Symp Quant Biol 78, 117-124 (2013). doi.org:10.1101/sqb.2013.78.021246
18 Tominaga, K. et al. IL-12 synergizes with IL-18 or IL-1beta for IFN-gamma production from human T cells. Int Immunol 12, 151-160 (2000). doi.org:10.1093/intimm/12.2.151
19 Pefia-Martinez, C., Rickman, A. D. & Heckmann, B. L. Beyond autophagy: LC3-associated phagocytosis and endocytosis. Sci Adv 8, eabn1702 (2022). doi.org:10.1 126/sciadv.abn1702
20 Cifone, M. G. et al. Dexamethasone-induced thymocyte apoptosis: apoptotic signal involves the sequential activation of phosphoinositide-specific phospholipase C, acidic sphingomyelinase, and caspases. Blood 93, 2282-2296 (1999).
21 Peng, Y. & Elkon, K. B. Autoimmunity in MFG-E8-deficient mice is associated with altered trafficking and enhanced cross-presentation of apoptotic cell antigens. The Journal of clinical investigation 121, 2221-2241 (2011). doi.org:10.1 172/JCI43254
22 Qin, W., Cho, K. F., Cavanagh, P. E. & Ting, A. Y. Deciphering molecular interactions by proximity labeling. Nature methods 18, 133-143 (2021). doi.org:10.1038/s41592-020-01010-5
23 Cho, K. F. et al. Proximity labeling in mammalian cells with TurboID and split-TurboID. Nature protocols 15, 3971-3999 (2020). doi.org:10.1038/s41596-020-0399-0
24 Simonsen, A. et al. Alfy, a novel FYVE-domain-containing protein associated with protein granules and autophagic membranes. Journal of cell science 117, 4239-4251 (2004). doi.org:10.1242/jcs.01287
25 Jönsson, F., Gurniak, C. B., Fleischer, B., Kirfel, G. & Witke, W. Immunological responses and actin dynamics in macrophages are controlled by N-cofilin but are independent from ADF. PloS one 7, e36034 (2012). doi.org:10.1371/journal.pone.0036034
26 Burger, D., Fickentscher, C., de Moerloose, P. & Brandt, K. J. F-actin dampens NLRP3 inflammasome activity via Flightless-I and LRRFIP2. Scientific reports 6, 29834 (2016). doi.org:10.1038/srep29834
27 Man, S. M. et al. Actin polymerization as a key innate immune effector mechanism to control Salmonella infection. Proceedings of the National Academy of Sciences of the United States of America 111, 17588-17593 (2014). doi.org:10.1073/pnas.1419925111
28 Joshi, H. et al. L-plastin enhances NLRP3 inflammasome assembly and bleomycin-induced lung fibrosis. Cell reports 38, 110507 (2022). doi.org:10.1016/j.celrep.2022.110507
29 Filimonenko, M. et al. The selective macroautophagic degradation of aggregated proteins requires the PI3P-binding protein Alfy. Molecular cell 38, 265-279 (2010). doi.org:10.1016/j.molcel.2010.04.007
30 Yamamoto, A. & Simonsen, A. Alfy-dependent elimination of aggregated proteins by macroautophagy: can there be too much of a good thing? Autophagy 7, 346-350 (2011).
31 Poon, I. K. H. & Ravichandran, K. S. Targeting Efferocytosis in Inflammaging. Annu Rev Pharmacol Toxicol (2023). doi.org:10.1146/annurev-pharmtox-032723-110507

### Example 5—Increasing Alfy Levels Protects Against CNS Changes in a Mouse Model of Autoimmunity

Systemic lupus erythematosis (SLE) can have repercussions system wide, with over 90% of patients developing neuropsychiatric SLE, which can lead to cognitive dysfunction, myelitis and stroke. Studies suggest that a cycle of progressive neuroinflammation and neuronal loss may contribute to the CNS related changes (1). To determine if increasing Alfy levels can protect CNS dysfunction, we determined if the autoimmune-like phenotypes associated with increasing the burden of apopotic cells (AC) extends to the CNS and is protected when Alfy levels are elevated.

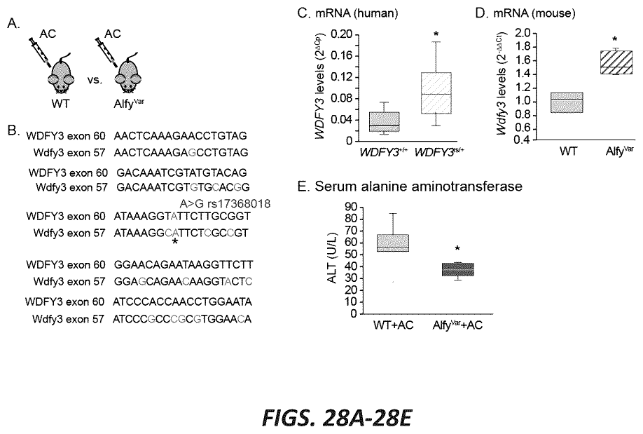

To achieve elevated Alfy levels, we turned to the Alfy Variant (AlfyVar) mice (FIG. 28A). AlfyVar mice carry an orthologue of a rare SNP that leads to increased Alfy expression (FIG. 28B-D). As we have shown previously using ectopic overexpression of Alfy (FIG. 26 and S4), knocking-in the SNP to the endogenous locus that encodes Alfy (Wdy3) was sufficient to diminish the elevated levels of alanine aminotransferase (ALT) that is caused by systemic AC injections in mice. (FIG. 28E) This outcome highlights two key observations: One, it confirms that under control conditions, the systemic administration of ACs are evoking a similar auto-immune response in these mice as we have described previously (FIG. 26 and S4); and two, that similarly to ectopic overexpression of Alfy, the elevated levels of Alfy in the Alfy^{Var} mice can attenuate the organ damage evoked by the auto-immune response.

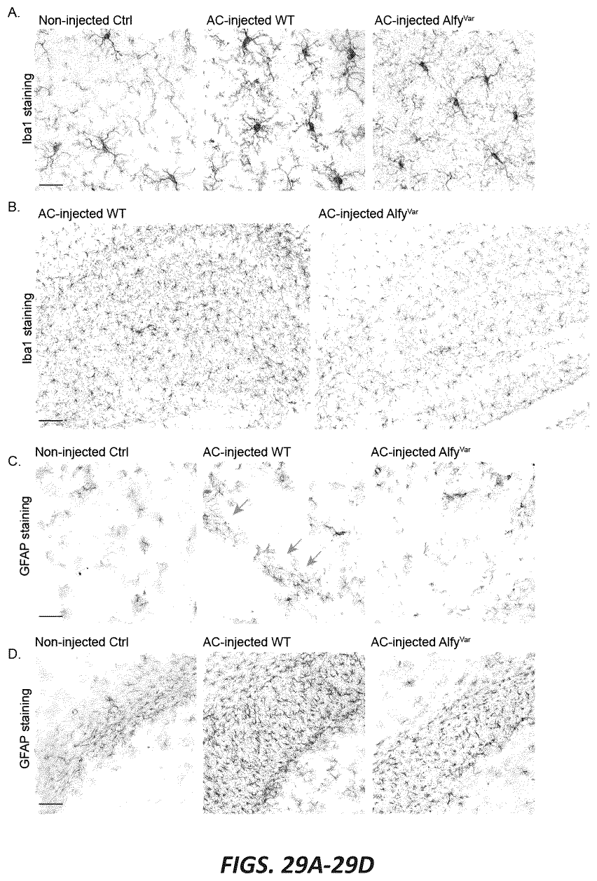

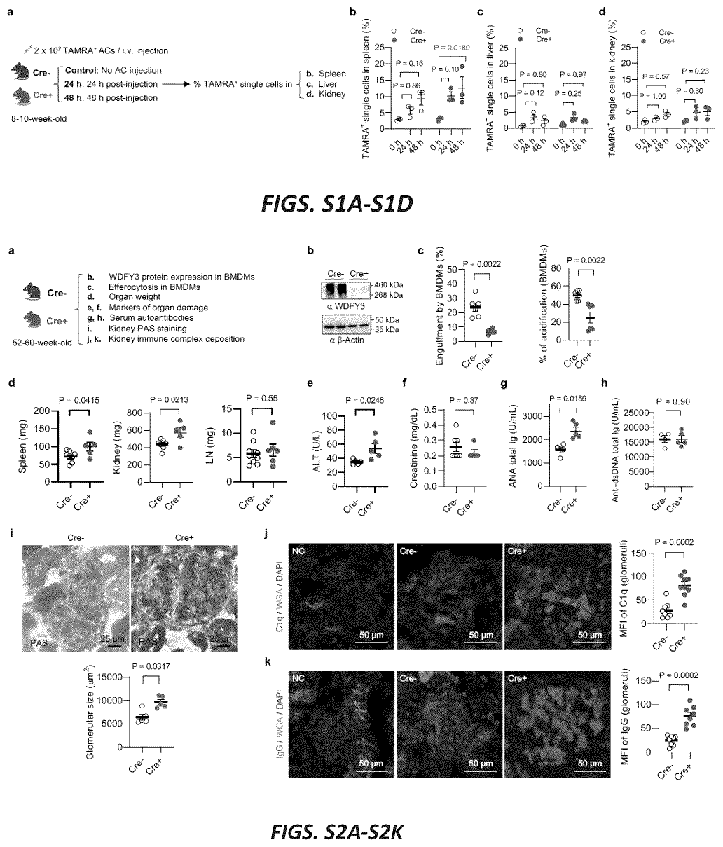

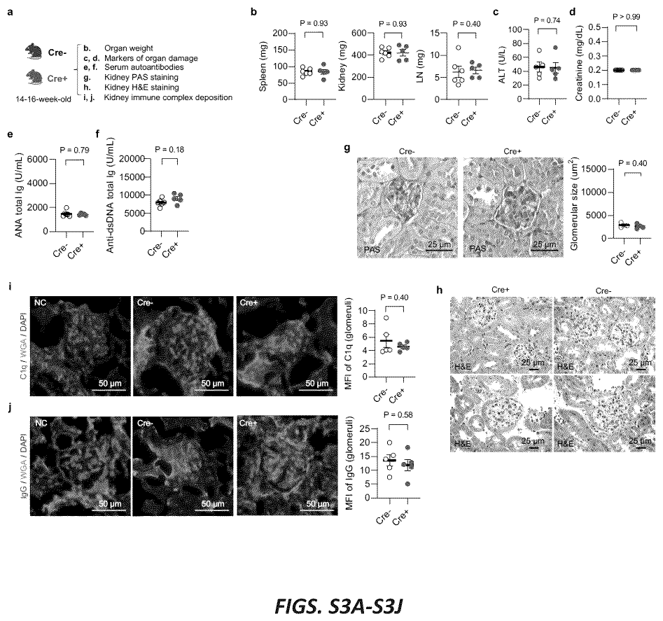

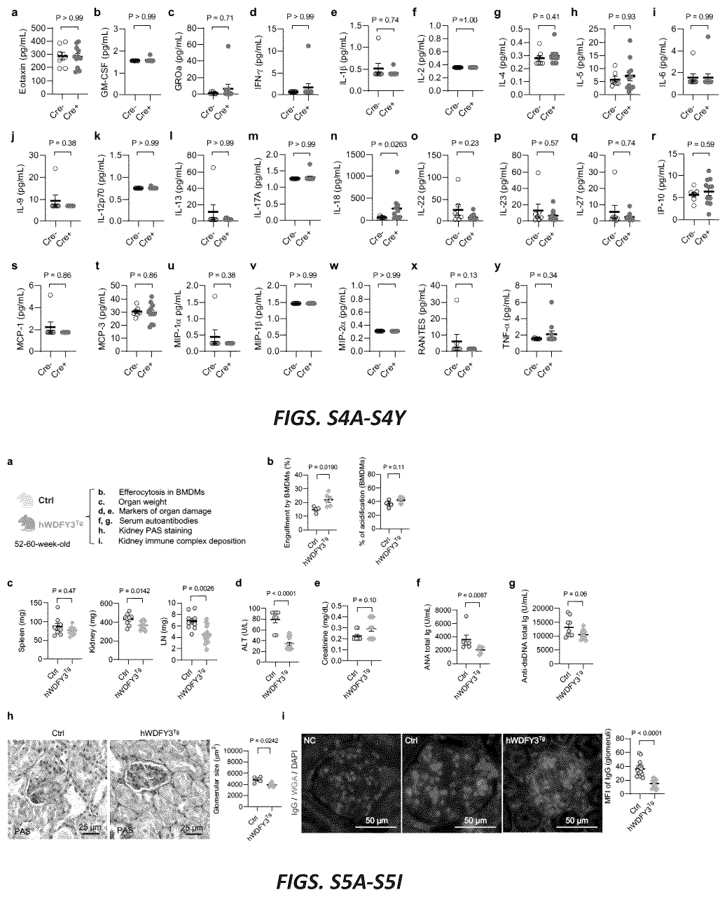

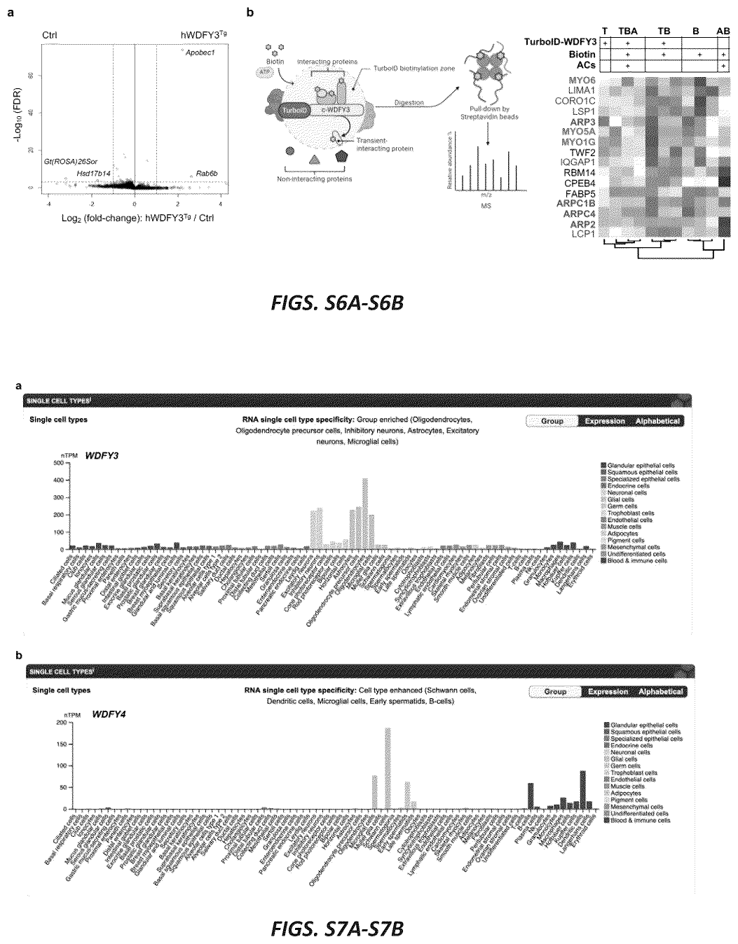

Given that we have an evoked response in the periphery, we next determined if these mice also demonstrated changes in the CNS. Immunohistochemical staining for Iba1 and GFAP revealed a profound impact of AC administration on neuroinflammation (FIG. 29). Compared to somatosensory cortex of non-injected control mice, AC-injected mice showed Iba1 positive microglia demonstrating a stress-primed morphology, with enhanced ramifications and multinucleation. The hippocampus of AC-injected WT mice was covered in Iba1 positive cells. GFAP staining similarly revealed a highly stressed state, including glial scarring in the striatum, and enhanced reactivity in the corpus callosum. In contrast, although some signs of neural stress were present, elevation of Alfy levels in the Alfy^{Var} mice markedly dampened the changes observed in both microglia and astrocytes. Taken together, these data show that elevated Alfy levels protect against the damaging impact of an evoked autoimmune response, providing a novel therapeutic approach to combat SLE and other autoimmune disorders that affect multiple organ systems.

### BIBLIOGRAPHY

1. Ballok, D. A. (2006) Neuroimmunopathology in a murine model of neuropsychiatric lupus. Brain Res Rev. 54(1):67-69.

All publications, patents and patent applications are incorporated herein by reference. While in the foregoing specification, this invention has been described in relation to certain embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A method to prevent, inhibit or treat an autoimmune disease in a mammal, comprising administering to the mammal a composition comprising an effective amount of isolated nucleic acid comprising a nucleotide sequence encoding WDFY3/Alfy or a portion thereof, a nucleotide sequence comprising a WDFY3/Alfy-specific long non-coding RNA (lncRNA) or a corresponding DNA sequence, a nucleotide sequence comprising a portion of a lncRNA WDFY3-AS2 or a corresponding DNA sequence, or isolated WDFY3/Alfy or a portion thereof.

2. The method of claim 1, wherein the mammal has or is at risk of having lupus, multiple sclerosis, myasthenia gravis, rheumatoid arthritis, reactive arthritis, rheumatoid arthritis, Sjogren syndrome, or Type I diabetes.

3. The method of claim 1, wherein the isolated nucleic acid is a vector.

4. The method of claim 3, wherein the vector is a viral vector or a set of viral vectors.

5. The method of claim 4, wherein the viral vector comprises an adeno-associated virus, adenovirus, lentivirus or a herpesvirus.

6. The method of claim 1, wherein the WDFY3/Alfy or portion thereof has at least one amino acid substitution that if present in full length WDFY3/Alfy results in a variant WDFY3/Alfy.

7. The method of claim 4, wherein the set of viral vectors each comprises a different portion of the coding region for WDFY3/Alfy.

8. The method of claim 7, wherein each portion is linked to a N-terminal or C-terminal intein.

9. The method of claim 7, wherein each portion having a coding region is flanked by a splice acceptor site or a splice donor site, or both.

10. The method of claim 1, wherein the composition comprises liposomes or nanoparticles.

11. The method of claim 1, wherein the isolated nucleic acid comprises any one of SEQ ID NOs: 11-14 or a nucleotide sequence with at least 80% nucleotide sequence identity thereto, or any combination thereof.

12. The method of claim 1, wherein the isolated nucleic acid comprises a portion of any one of SEQ ID NOs: 11-14, or any combination thereof, with the same activity as one of SEQ ID NOs: 11-14.

13. The method of claim 1, wherein the portion of nucleic acid is in the first 1000, 500 or 250 nucleotides of SEQ ID NO: 3.

14. The method of claim 1, wherein the portion is in the last 500 or 250 nucleotides of SEQ ID NO:3.

15. A method to prevent, inhibit or treat a vascular or cardiovascular disorder in a mammal, comprising administering to the mammal a composition comprising an effective amount of isolated nucleic acid comprising a nucleotide sequence encoding WDFY3/Alfy or a portion thereof, a nucleotide sequence comprising a WDFY3/Alfy-specific long non-coding RNA (lncRNA) or a corresponding DNA sequence, a nucleotide sequence comprising a portion of a lncRNA WDFY3-AS2 or a corresponding DNA sequence, or isolated WDFY3/Alfy or a portion thereof.

16. The method of claim 15, wherein the disorder is a cardiovascular disorder.

17. The method of claim 15, wherein the mammal has coronary artery disease or atherosclerosis.

18. The method of claim 15, wherein the nucleic acid is a vector.

19. The method of claim 18, wherein the vector is a viral vector or a set of viral vectors.

20. The method of claim 19, wherein the viral vector comprises an adeno-associated virus, adenovirus, lentivirus or a herpesvirus.

21. The method of claim 15, wherein the composition comprises liposomes or nanoparticles.

22. The method of claim 15, wherein the isolated nucleic acid comprises any one of SEQ ID NOs: 11-14 or a nucleotide sequence with at least 80% nucleotide sequence identity thereto, or any combination thereof.

23. The method of claim 15, wherein the isolated nucleic acid comprises a portion of any one of SEQ ID NOs: 11-14, or any combination thereof, with the same activity as one of SEQ ID NOs: 11-14.
